(19) United States
(12) Patent Application Publication
(10) Pub. No.: US 2018/0265902 A1
(43) Pub. Date: Sep. 20, 2018

(54) Beta-Alanine Aminotransferases For The Production of 3-Hydroxypropionic Acid

(71) Applicant: Novozymes A/S, Bagsvaerd (DK)

(72) Inventors: Monica Tassone, West Sacramento, CA (US); Audrey Diano, Vacaville, CA (US)

(73) Assignee: Novozymes A/S, Bagsvaerd (DK)

(21) Appl. No.: 15/754,402

(22) PCT Filed: Aug. 24, 2016
(86) PCT No.: PCT/US2016/048474

§ 371 Date: Feb. 22, 2018

Related U.S. Application Data
(60) Provisional application No. 62/209,260, filed on Aug. 24, 2015.

(51) Int. Cl. C12P 7/42; C12N 9/10; C07C 51/377; C07C 67/08; C07C 29/149

(52) U.S. Cl. CPC C12P 7/42; C12N 9/1096; C07C 29/149; C07C 51/377; C07C 67/08; C12Y 206/01019

(57) ABSTRACT

Provided herein are recombinant host cells having an active 3-Hydroxypropionic Acid (3-HP) pathway wherein the host cells comprise a heterologous polynucleotide encoding an aminotransferase of E.C. 2.6.1.19 and wherein the aminotransferase comprises the motif (A,C)-Xn-(GSLS)-Xn-SKA(L/l)(L/l). Also described are methods of making the host cells, and methods using the cells to produce 3-HP and derivatives of 3-HP (e.g., acrylic acid).

### CROSS-REFERENCE TO RELATED APPLICATIONS

This application claims priority benefit of U.S. provisional application Ser. No. 62/209,260, filed on Aug. 24, 2015. The content of this application is fully incorporated herein by reference.

### REFERENCE TO A SEQUENCE LISTING

This application contains a Sequence Listing in computer readable form, which is incorporated herein by reference.

### BACKGROUND

3-hydroxypropionic acid (3-HP) is a three carbon carboxylic acid identified by the U.S. Department of Energy as one of the top 12 high-potential building block chemicals that can be made by fermentation. Alternative names for 3-HP, which is an isomer of lactic (2-hydroxypropionic) acid, include ethylene lactic acid and 3-hydroxypropionate. 3-HP is an attractive renewable platform chemical, with 100% theoretical yield from glucose, multiple functional groups that allow it to participate in a variety of chemical reactions, and low toxicity. 3-HP can be used as a substrate to form several commodity chemicals, such as 1,3-propanediol, malonic acid, acrylamide, and acrylic acid. Acrylic acid is a large-volume chemical (>7 billion lbs/year) used to make acrylate esters and superabsorbent polymers, and is currently derived from catalytic oxidation of propylene. Fermentative production of 3-HP would provide a sustainable alternative to petrochemicals as the feedstock for these commercially-significant chemicals, thus reducing energy consumption, dependence on foreign oil supplies, and the production of greenhouse gases.

β-alanine aminotransferase (BAAT) is an enzyme that converts β-alanine to malonate semialdehyde, the precursor to 3-HP (FIG. 1). The BAAT enzyme is believed to have similar origins to 4-aminobutyrate aminotransferases (gabT) (Andersen et al., 2007, FEBS J 274: 1804-1817). While BAAT/gabT aminotransferase homologs are known, it was not clear, prior to Applicant's disclosure below, which amino acids and/or motifs are responsible for the respective BAAT and gabT activities. It would be advantageous with some engineered 3-HP metabolic pathways to specifically express enzymes that are known to have BAAT activity.

### SUMMARY

Described herein are recombinant host cells having an active 3-HP pathway, wherein the recombinant cells comprise a heterologous polynucleotide encoding an aminotransferase of E.C. 2.6.1.19, and wherein the cells are capable of producing 3-HP. In some embodiments, the aminotransferase comprises the motif (A,C)-Xn-(GSLS)-Xn-SKA(L/l)(L/l). In some embodiments, the aminotransferase is other than a S. kluyveri, I. orientalis, or S. avermitilis aminotransferase. In some embodiments, the aminotransferase is a β-alanine aminotransferase (BAAT). In some embodiments, the aminotransferase comprises (i) AC at a positions corresponding to positions 103-104 of SEQ ID NO: 20; (ii) GSLS at positions corresponding to positions 194-197 of SEQ ID NO: 20; and/or (ii) SKA(L/l)(L/l) at positions corresponding to positions 354-358 of SEQ ID NO: 20.

In some embodiments, the aminotransferase (e.g., BAAT) has at least 60% sequence identity to SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234.

In some embodiments, the recombinant cells comprise an active 3-HP pathway that proceeds through a β-alanine and/or malonate semialdehyde intermediate.

In some embodiments, the recombinant cells produce a greater amount of 3-HP compared to the cells without the heterologous polynucleotide encoding the encoding the aminotransferase (e.g., BAAT) when cultivated under identical conditions.

In some aspects, the recombinant cells comprise one or more (e.g., two, several) heterologous polynucleotides selected from a heterologous polynucleotide encoding a PYC; a heterologous polynucleotide encoding an AAT; a heterologous polynucleotide encoding an ADC; a heterologous polynucleotide encoding a 3-HPDH; and a heterologous polynucleotide encoding a PPC. In some embodiments, the recombinant cells comprise a heterologous polynucleotide encoding an aspartate 1-decarboxylase (ADC).

In some embodiments, the recombinant cells comprise a disruption to one or more endogenous genes encoding a PDC, ADH, GAL6, CYB2A, CYB2B, GPD, GPP, ALD, or PCK. In some embodiments, the recombinant cells comprise a disruption to one or both of an endogenous gene encoding a PDC and an endogenous gene encoding a GPD.

In some embodiments, the recombinant cells are bacterial or fungal cells. In some embodiments, the recombinant cells are yeast cells such as Issatchenkia, Candida, Kluyveromyces, Pichia, Schizosaccharomyces, Torulaspora, Zygosaccharomyces, or Saccharomyces yeast cells. In some embodiments, the recombinant cells are 3-HP-resistant yeast cells. In some embodiments, the recombinant cells are unable to ferment pentose sugars.

Also described are methods of producing 3-HP and related compounds. In one aspect is a method of producing 3-HP, comprising: (a) cultivating a recombinant cell described herein in a medium under suitable conditions to produce 3-HP; and (b) recovering the 3-HP. In another aspect is a method of producing acrylic acid or a salt thereof, comprising: (a) cultivating a recombinant cell described herein in a medium under suitable conditions to produce 3-HP; (b) recovering the 3-HP; (c) dehydrating the 3-HP under suitable conditions to produce acrylic acid or a salt thereof; and (d) recovering the acrylic acid or salt thereof.

### BRIEF DESCRIPTION OF THE FIGURES

FIG. 1 shows a summary of select 3-HP pathways from glucose.

FIG. 2 shows a representative multiple sequence alignment (MSA) for the I. orientalis aminotransferase of SEQ ID NO: 20, S. kluyveri aminotransferase of SEQ ID NO: 21, I. orientalis aminotransferase of SEQ ID NO: 211, S. kluyveri aminotransferase of SEQ ID NO: 212, C. arabinofermentans aminotransferase of SEQ ID NO: 207, B. bruxellensis aminotransferase of SEQ ID NO: 208, and P. membranifaciens aminotransferase of SEQ ID NO: 206 (top→bottom). Regions for the motif (A,C)-Xn-(GSLS)-Xn-SKA(L/l)(L/l) are circled.

FIG. 3 shows molecular model of the I. orientalis BAAT dimer (SEQ ID NO: 20) showing conserved positions 103-104 and 194-197 near the active site.

FIG. 4 shows a molecular model of the I. orientalis BAAT dimer (SEQ ID NO: 20) showing positions 354-358.

FIG. 5 shows a plasmid map for pMcTs343.

FIG. 6 shows a plasmid map for pMcTs351.

FIG. 7 shows a plasmid map for pMcTs352.

FIG. 8 shows a plasmid map for pMcTs356.

FIG. 9 shows a plasmid map for pMcTs342.

FIG. 10 shows a plasmid map for pMBin211.

FIG. 11 shows a plasmid map for pMBin218.

### DEFINITIONS

3-HP: The term “3-HP” includes salt and acid forms of “3-hydroxypropionic acid” unless stated otherwise from the context in which it is used.

Abbreviations: 3-HPA, 3-hydroxypropionaldehyde; 3-HPDH, 3-hydroxypropionic acid dehydrogenase; AAM, alanine 2,3-aminomutase; AAT, aspartate aminotransferase; ACC, acetyl-CoA carboxylase; ADC, aspartate 1-decarboxylase; AKG, alpha-ketoglutarate; ALD, aldehyde dehydrogenase; BAAT, β-alanine aminotransferase; BCKA, branched-chain alpha-keto acid decarboxylase; bp, base pairs; CYB2, L-(+)-lactate-cytochrome c oxidoreductase; CYC, iso-2-cytochrome c; EMS, ethane methyl sulfonase; ENO, enolase; gabT, 4-aminobutyrate aminotransferase; GAPDH, glyceraldehyde-3-phosphate dehydrogenase 3; GPD, glycerol 3-phosphate dehydrogenase; GPP, glycerol 3-phosphate phosphatase; HIBADH, 3-hydroxyisobutyrate dehydrogenase; IPDA, indolepyruvate decarboxylase; KGD, alpha-ketoglutarate decarboxylase; LDH, lactate dehydrogenase; MAE, malic enzyme; MDHB, malate dehydrogenase B; OAA, oxaloacetate; PCK, phosphoenolpyruvate carboxykinase; PDC, pyruvate decarboxylase; PDH, pyruvate dehydrogenase; PEP, phosphoenolpyruvate; PGK, phosphoglycerate kinase; PPC, phosphoenolpyruvate carboxylase; PYC, pyruvate carboxylase; RKI, ribose 5-phosphate ketol-isomerase; TAL, transaldolase; TEF1, translation elongation factor-1; TEF2, translation elongation factor-2; TKL, transketolase, XDH, xylitol dehydrogenase; XR, xylose reductase; YP, yeast extract/peptone.

Active 3-HP pathway: As used herein, a host cell having an “active 3-HP pathway” produces active enzymes necessary to catalyze each reaction of a metabolic pathway in a sufficient amount to produce 3-HP from a fermentable sugar, and therefore is capable of producing 3-HP in measurable yields when cultivated under fermentation conditions in the presence of at least one fermentable sugar. A host cell having an active 3-HP pathway comprises one or more 3-HP pathway genes. A “3-HP pathway gene” as used herein refers to a gene that encodes an enzyme involved in an active 3-HP pathway.

The active enzymes necessary to catalyze each reaction in an active 3-HP pathway may result from activities of endogenous gene expression, activities of heterologous gene expression, or from a combination of activities of endogenous and heterologous gene expression, as described in more detail herein.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Aminotransferase: As used herein, the term “aminotransferase” is defined as a β-alanine aminotransferase (BAAT) and/or 4-aminobutyrate aminotransferase (gabT) of E.C. 2.6.1.19. A “β-alanine aminotransferase” (BAAT) refers to an enzyme that has the ability to catalyze the conversion of β-alanine (BAL) to malonate semialdehyde. A pyd4 gene is sometimes referred to as encoding a BAAT enzyme. A “4-aminobutyrate aminotransferase” (gabT) refers to an enzyme that has the ability to catalyze the conversion of 4-aminobutyrate (GABA) to succinate semialdehyde. A uga1 gene is sometimes referred to as encoding a gabT enzyme. BAAT and gabT activities can be determined from cell-free extracts as described in the art, e.g., as described in Andersen et al., 2007, FEBS J 274: 1804-1817, or in the Examples section below. In some instances, an amino transferase may have one or both of BAAT and gabT activities.

Coding sequence: The term “coding sequence” or “coding region” means a polynucleotide sequence, which specifies the amino acid sequence of a polypeptide. The boundaries of the coding sequence are generally determined by an open reading frame, which usually begins with the ATG start codon or alternative start codons such as GTG and TTG and ends with a stop codon such as TAA, TAG, and TGA. The coding sequence may be a sequence of genomic DNA, cDNA, a synthetic polynucleotide, and/or a recombinant polynucleotide.

Control sequence: The term “control sequence” means a nucleic acid sequence necessary for polypeptide expression. Control sequences may be native or foreign to the polynucleotide encoding the polypeptide, and native or foreign to each other. Such control sequences include, but are not limited to, a leader sequence, polyadenylation sequence, propeptide sequence, promoter sequence, signal peptide sequence, and transcription terminator sequence. The control sequences may be provided with linkers for the purpose of introducing specific restriction sites facilitating ligation of the control sequences with the coding region of the polynucleotide encoding a polypeptide.

Disruption: The term “disruption” means that a coding region and/or control sequence of a referenced gene is partially or entirely modified (such as by deletion, insertion, and/or substitution of one or more nucleotides) resulting in the absence (inactivation) or decrease in expression, and/or the absence or decrease of enzyme activity of the encoded polypeptide. The effects of disruption can be measured using techniques known in the art such as detecting the absence or decrease of enzyme activity using from cell-free extract measurements referenced herein; or by the absence or decrease of corresponding mRNA (e.g., at least 25% decrease, at least 50% decrease, at least 60% decrease, at least 70% decrease, at least 80% decrease, or at least 90% decrease); the absence or decrease in the amount of corresponding polypeptide having enzyme activity (e.g., at least 25% decrease, at least 50% decrease, at least 60% decrease, at least 70% decrease, at least 80% decrease, or at least 90% decrease); or the absence or decrease of the specific activity of the corresponding polypeptide having enzyme activity (e.g., at least 25% decrease, at least 50% decrease, at least 60% decrease, at least 70% decrease, at least 80% decrease, or at least 90% decrease). Disruptions of a particular gene of interest can be generated by methods known in the art, e.g., by directed homologous recombination (see Methods in Yeast Genetics (1997 edition), Adams, Gottschling, Kaiser, and Stems, Cold Spring Harbor Press (1998)).

Endogenous gene: The term “endogenous gene” means a gene that is native to the referenced host cell. “Endogenous gene expression” means expression of an endogenous gene.

Expression: The term “expression” includes any step involved in the production of the polypeptide including, but not limited to, transcription, post-transcriptional modification, translation, post-translational modification, and secretion. Expression can be measured—for example, to detect increased expression—by techniques known in the art, such as measuring levels of mRNA and/or translated polypeptide.

Expression vector: The term “expression vector” means a linear or circular DNA molecule that comprises a polynucleotide encoding a polypeptide and is operably linked to control sequences, wherein the control sequences provide for expression of the polynucleotide encoding the polypeptide. At a minimum, the expression vector comprises a promoter sequence, and transcriptional and translational stop signal sequences.

Fermentable medium: The term “fermentable medium” or “fermentation medium” refers to a medium comprising one or more (e.g., two, several) sugars, such as glucose, fructose, sucrose, cellobiose, xylose, xylulose, arabinose, mannose, galactose, and/or soluble oligosaccharides, wherein the medium is capable, in part, of being converted (fermented) by a host cell into a desired product, such as 3-HP. In some instances, the fermentation medium is derived from a natural source, such as sugar cane, starch, or cellulose, and may be the result of pretreating the source by enzymatic hydrolysis (saccharification).

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

Host cell: The term “host cell” means any cell type that is susceptible to transformation, transfection, transduction, and the like with a nucleic acid construct or expression vector. The term “host cell” encompasses any progeny of a parent cell that is not identical to the parent cell due to mutations that occur during replication. The term “recombinant cell” is defined herein as a non-naturally occurring host cell comprising one or more (e.g., two, several) heterologous polynucleotides. The host cell may be a mutant strain described herein, or further disrupted to provide a mutant strain described herein.

Hybridization conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 0.2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 0.2×SSC, 0.2% SDS at 70° C.

Isolated: The term “isolated” means a substance in a form or environment that does not occur in nature. Non-limiting examples of isolated substances include (1) any non-naturally occurring substance, (2) any substance including, but not limited to, any host cell, enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated.

Mutant: The term “mutant” means the resulting strain after one or more disruptions are made to a parent strain.

Nucleic acid construct: The term “nucleic acid construct” means a polynucleotide comprises one or more (e.g., two, several) control sequences. The polynucleotide may be single-stranded or double-stranded, and may be isolated from a naturally occurring gene, modified to contain segments of nucleic acids in a manner that would not otherwise exist in nature, or synthetic.

Operably linked: The term “operably linked” means a configuration in which a control sequence is placed at an appropriate position relative to the coding sequence of a polynucleotide such that the control sequence directs expression of the coding sequence.

Sequence Identity: The relatedness between two amino acid sequences or between two nucleotide sequences is described by the parameter “sequence identity”.

For purposes described herein, the degree of sequence identity between two amino acid sequences is determined using the Needleman-Wunsch algorithm (Needleman and Wunsch, J. Mol. Biol. 1970, 48, 443-453) as implemented in the Needle program of the EMBOSS package (EMBOSS: The European Molecular Biology Open Software Suite, Rice et al., Trends Genet 2000, 16, 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of the Referenced Sequence−Total Number of Gaps in Alignment)

For purposes described herein, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Referenced Sequence−Total Number of Gaps in Alignment)

Volumetric productivity: The term “volumetric productivity” refers to the amount of referenced product produced (e.g., the amount of 3-HP produced) per volume of the system used (e.g., the total volume of media and contents therein) per unit of time.

Reference to “about” a value or parameter herein includes aspects that are directed to that value or parameter per se. For example, description referring to “about X” includes the aspect “X”. When used in combination with measured values, “about” includes a range that encompasses at least the uncertainty associated with the method of measuring the particular value, and can include a range of plus or minus two standard deviations around the stated value.

As used herein and in the appended claims, the singular forms “a,” “or,” and “the” include plural referents unless the context clearly dictates otherwise. It is understood that the aspects described herein include “consisting” and/or “consisting essentially of” aspects.

Unless defined otherwise or clearly indicated by context, all technical and scientific terms used herein have the same meaning as commonly understood by one of ordinary skill in the art.

### DETAILED DESCRIPTION

Described herein, inter alia, are recombinant cells having an active 3-HP pathway which comprise a heterologous polynucleotide encoding a aminotransferase of E.C. 2.6.1.19, wherein the aminotransferase has β-alanine aminotransferase (BAAT) activity. While aminotransferases of E.C. 2.6.1.19 are known to have either β-alanine aminotransferase (BAAT) activity, 4-aminobutyrate aminotransferase (gabT) activity, or both BAAT and gabT activity (Anderson et al, FEBS 2007, 274, 1804-1817), it was unclear which regions of these enzymes are responsible for activity toward the β-alanine and/or GABA substrate.

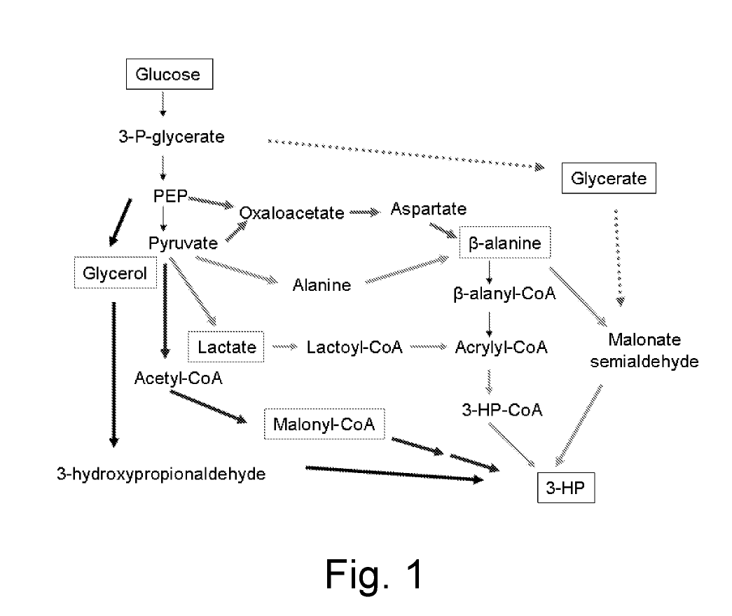

As described in the examples below, the Applicant has identified three putative regions of aminotransterase enzymes unique to those aminotransferases having β-alanine aminotransferase (BAAT) activity: (i) AC at a positions corresponding to positions 103-104 of SEQ ID NO: 20; (ii) GSLS at positions corresponding to positions 194-197 of SEQ ID NO: 20; and (iii) SKA(L/l)(L/l) at positions corresponding to positions 354-358 of SEQ ID NO: 20. Because the (A,C)-Xn-(GSLS)-Xn-SKA(L/l)(L/l) motif is unique to β-alanine aminotransferases (BAAT) and absent in 4-aminobutyrate aminotransferases (gabT), the Applicant has taught the skilled artisan which enzymes—based on sequence analysis to identify the motif—may be used in an active 3-HP pathway which converts β-alanine (BAL) to malonate semialdehyde (See, FIG. 1).

The aminotransferase (e.g., BAAT) can be any aminotransferase comprising the motif(s) described herein and/or related to the sequences described herein that is suitable for the host cells and their methods of use, such as the aminotransferases described by specific SEQ ID numbers, or variants thereof that retain aminotransferase activity (such as artificial variants or a natural variants from other species).

Accordingly, in one aspect, the heterologous polynucleotide encodes an aminotransferase of E.C. 2.6.1.19, wherein the aminotransferase (a) comprises the motif (A,C)-Xn-(GSLS)-Xn-SKA(L/l)(L/l); and (b) has β-alanine aminotransferase (BAAT) activity. In some embodiments, the aminotransferase is other than a S. kluyveri, I. orientalis, or S. avermitilis aminotransferase.

In some embodiments, the aminotransferase comprises: (i) AC at a positions corresponding to positions 103-104 of SEQ ID NO: 20; (ii) GSLS at positions corresponding to positions 194-197 of SEQ ID NO: 20; and/or (iii) SKA(L/l)(L/l) at positions corresponding to positions 354-358 of SEQ ID NO: 20. For purposes of the present invention, the sequence of SEQ ID NO: 20 is used to determine the corresponding amino acid residue in another aminotransferase. The amino acid sequence of another aminotransferase is aligned with SEQ ID NO: 20, and based on the alignment, the amino acid position number corresponding to any amino acid residue in SEQ ID NO: 20 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are a gap open penalty of 10, a gap extension penalty of 0.5, and the EBLOSUM62 (EMBOSS version of BLOSUM62) substitution matrix. Numbering of the amino acid positions is based on the full-length polypeptide of SEQ ID NO: 20 wherein position 1 is the first amino acid of the peptide (i.e., Met). For example, based on alignment, the position corresponding to the alanine at position 103 of SEQ ID NO: 4 corresponds to the alanine at position 103 of the Pichia membranifaciens BAAT of SEQ ID NO: 206, and to the alanine at position 104 of Candida guilliermondii of SEQ ID NO: 207.

In some aspects, the aminotransferase (e.g., BAAT comprising the (A,C)-Xn-(GSLS)-Xn-SKA(L/l)(L/l) motif) has at least 20%, e.g., at least 40%, at least 50%, at least 60%, at least 70%, at least 80%, at least 90%, at least 95%, at least 96%, at least 97%, at least 98%, at least 99%, or 100% of the aminotransferase activity (e.g., BAAT activity) of SEQ ID NO: 20, SEQ ID NO: 21, SEQ ID NO: 22, SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234 under the same conditions.

In some embodiments the aminotransferase has gabT activity in addition to BAAT activity, meaning that it has activity on 4-aminobutyrate (GABA) as well as β-alanine (BAL). In some embodiments, the aminotransferase is derived from a native gabT gene (e.g., by random or directed engineering) to encode a polypeptide with BAAT activity. In some embodiments, the aminotransferase has an increased specificity for β-alanine (BAL) compared to 4-aminobutyrate (GABA) (e.g., greater than 2-fold, 5-fold, 10-fold, 20-fold, 50-fold, 100-fold, 200-fold, 500-fold, or 1000-fold specificity for β-alanine compared to GABA).

In some embodiments, the recombinant cells comprise an active 3-HP pathway that proceeds through a β-alanine and/or malonate semialdehyde intermediate. In some aspects, the cells lack an endogenous BAAT gene or have a disruption of an endogenous BAAT. In one aspect, BAAT is present in the cytosol of the host cells.

In one embodiment, the heterologous polynucleotide encodes an aminotransferase (e.g., BAAT comprising the (A,C)-Xn-(GSLS)-Xn-SKA(L/l)(L/l) motif) that comprises or consists of the amino acid sequence of SEQ ID NO: 206. In another embodiment, the aminotransferase comprises or consists of the amino acid sequence of SEQ ID NO: 207. In another embodiment, the aminotransferase comprises or consists of the amino acid sequence of SEQ ID NO: 208. In another embodiment, the aminotransferase comprises or consists of the amino acid sequence of SEQ ID NO: 209. In another embodiment, the aminotransferase comprises or consists of the amino acid sequence of SEQ ID NO: 218. In another embodiment, the aminotransferase comprises or consists of the amino acid sequence of SEQ ID NO: 220. In another embodiment, the aminotransferase comprises or consists of the amino acid sequence of SEQ ID NO: 222. In another embodiment, the aminotransferase comprises or consists of the amino acid sequence of SEQ ID NO: 224. In another embodiment, the aminotransferase comprises or consists of the amino acid sequence of SEQ ID NO: 226. In another embodiment, the aminotransferase comprises or consists of the amino acid sequence of SEQ ID NO: 228. In another embodiment, the aminotransferase comprises or consists of the amino acid sequence of SEQ ID NO: 230. In another embodiment, the aminotransferase comprises or consists of the amino acid sequence of SEQ ID NO: 232. In another embodiment, the aminotransferase comprises or consists of the amino acid sequence of SEQ ID NO: 234.

In another embodiment, the aminotransferase lacks an N-terminal mitochondrial target sequence (MTS). Enzyme mitochondrial target sequences may be identified using computational programs known in the art, such as Target P (Emanuelsson et al, Nat Protoc 2, 953-971 (2007)) and MitoProt (Claros et al, Eur. J. Biochem. 241, 779-786 (1996)).

In another embodiment, the heterologous polynucleotide encodes a fragment of a polypeptide of SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234, wherein the fragment has aminotransferase activity (e.g., BAAT activity). In one embodiment, the number of amino acid residues in the fragment is at least 75%, e.g., at least 80%, 85%, 90%, or 95% of the number of amino acid residues in the referenced sequence.

The aminotransferase may also be a variant of the described aminotransferases. In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 206. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 206. In one embodiment, the aminotransferase comprises or consists of the amino acid sequence of SEQ ID NO: 206, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 206. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 207. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 207. In one embodiment, the aminotransferase comprises or consists of the amino acid sequence of SEQ ID NO: 207, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 207. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 208. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 208. In one embodiment, the aminotransferase comprises or consists of the amino acid sequence of SEQ ID NO: 208, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 208. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 209. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 209. In one embodiment, the aminotransferase comprises or consists of the amino acid sequence of SEQ ID NO: 209, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 209. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 218. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 218. In one embodiment, the aminotransferase comprises or consists of the amino acid sequence of SEQ ID NO: 218, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 218. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 220. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 220. In one embodiment, the aminotransferase comprises or consists of the amino acid sequence of SEQ ID NO: 220, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 220. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 222. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 222. In one embodiment, the aminotransferase comprises or consists of the amino acid sequence of SEQ ID NO: 222, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 222. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 224. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 224. In one embodiment, the aminotransferase comprises or consists of the amino acid sequence of SEQ ID NO: 224, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 224. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 226. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 226. In one embodiment, the aminotransferase comprises or consists of the amino acid sequence of SEQ ID NO: 226, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 226. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 228. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 228. In one embodiment, the aminotransferase comprises or consists of the amino acid sequence of SEQ ID NO: 228, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 228. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 230. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 230. In one embodiment, the aminotransferase comprises or consists of the amino acid sequence of SEQ ID NO: 230, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 230. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 232. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 232. In one embodiment, the aminotransferase comprises or consists of the amino acid sequence of SEQ ID NO: 232, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 232. In some embodiments, the total number of amino acid substitutions, deletions and/or insertions is not more than 10, e.g., not more than 9, 8, 7, 6, 5, 4, 3, 2, or 1.

In one embodiment, the aminotransferase (e.g., BAAT comprising the (A,C)-Xn-(GSLS)-Xn-SKA(L/l)(L/l) motif) has at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 234. In one embodiment, the aminotransferase sequence differs by no more than ten amino acids, e.g., by no more than five amino acids, by no more than four amino acids, by no more than three amino acids, by no more than two amino acids, or by one amino acid from SEQ ID NO: 234. In one embodiment, the aminotransferase comprises or consists of the amino acid sequence of SEQ ID NO: 234, or an allelic variant, or a fragment thereof having aminotransferase activity (e.g., BAAT activity). In one embodiment, the aminotransferase has an amino acid substitution, deletion, and/or insertion of one or more (e.g., two, several) amino acids of SEQ ID NO: 234. In some embodiments, the total number of amino acid substitutions, deletions and/or insertions is not more than 10, e.g., not more than 9, 8, 7, 6, 5, 4, 3, 2, or 1.

The amino acid changes are generally of a minor nature, that is conservative amino acid substitutions or insertions that do not significantly affect the folding and/or activity of the protein; small deletions, typically of one to about 30 amino acids; small amino-terminal or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the aminotransferase, alter the substrate specificity, change the pH optimum, and the like.

Essential amino acids can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for aminotransferase activity to identify amino acid residues that are critical to the activity of the molecule. See also, Hilton et al., 1996, J. Biol. Chem. 271: 4699-4708. The active site of the aminotransferase or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identities of essential amino acids can also be inferred from analysis of identities with other aminotransferases that are related to the referenced aminotransferase.

Additional guidance on the structure-activity relationship of the aminotransferases herein can be determined using multiple sequence alignment (MSA) techniques well-known in the art. Based on the teachings herein, the skilled artisan could make similar alignments with any number of aminotransferases described herein or known in the art. Such alignments aid the skilled artisan to determine potentially relevant domains (e.g., binding domains or catalytic domains), as well as which amino acid residues are conserved and not conserved among the different aminotransferase sequences. It is appreciated in the art that changing an amino acid that is conserved at a particular position between the disclosed aminotransferases will more likely result in a change in biological activity (Bowie et al., 1990, Science 247: 1306-1310: “Residues that are directly involved in protein functions such as binding or catalysis will certainly be among the most conserved”). In contrast, substituting an amino acid that is not highly conserved among the aminotransferases will not likely or significantly alter the biological activity.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active aminotransferases can be recovered from the host cells and rapidly sequenced using standard methods in the art. These methods allow the rapid determination of the importance of individual amino acid residues in a polypeptide.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 239. In one embodiment, the heterologous polynucleotide encoding the aminotransferase comprises the coding sequence of SEQ ID NO: 239.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 240. In one embodiment, the heterologous polynucleotide encoding the aminotransferase comprises the coding sequence of SEQ ID NO: 240.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 241. In one embodiment, the heterologous polynucleotide encoding the aminotransferase comprises the coding sequence of SEQ ID NO: 241.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 242. In one embodiment, the heterologous polynucleotide encoding the aminotransferase comprises the coding sequence of SEQ ID NO: 242.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 243. In one embodiment, the heterologous polynucleotide encoding the aminotransferase comprises the coding sequence of SEQ ID NO: 243.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 244. In one embodiment, the heterologous polynucleotide encoding the aminotransferase comprises the coding sequence of SEQ ID NO: 244.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 245. In one embodiment, the heterologous polynucleotide encoding the aminotransferase comprises the coding sequence of SEQ ID NO: 245.

In another embodiment, In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 246. In one embodiment, the heterologous polynucleotide encoding the aminotransferase comprises the coding sequence of SEQ ID NO: 246.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 247. In one embodiment, the heterologous polynucleotide encoding the aminotransferase comprises the coding sequence of SEQ ID NO: 247.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 248. In one embodiment, the heterologous polynucleotide encoding the aminotransferase comprises the coding sequence of SEQ ID NO: 248.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 249. In one embodiment, the heterologous polynucleotide encoding the aminotransferase comprises the coding sequence of SEQ ID NO: 249.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 250. In one embodiment, the heterologous polynucleotide encoding the aminotransferase comprises the coding sequence of SEQ ID NO: 250.

In another embodiment, the heterologous polynucleotide encoding the aminotransferase (e.g., BAAT comprising the (A,C)-Xn-(GSLS)-Xn-SKA(L/l)(L/l) motif) comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 251. In one embodiment, the heterologous polynucleotide encoding the aminotransferase comprises the coding sequence of SEQ ID NO: 251.

In another embodiment, the heterologous polynucleotide encoding the aminotransferase (e.g., BAAT comprising the (A,C)-Xn-(GSLS)-Xn-SKA(L/l)(L/l) motif) comprises a subsequence of the coding sequence of SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251, wherein the subsequence encodes a polypeptide having aminotransferase activity (e.g., BAAT activity). In another embodiment, the number of nucleotides residues in the coding subsequence is at least 75%, e.g., at least 80%, 85%, 90%, or 95% of the number of the referenced coding sequence.

The referenced coding sequence of any related aspect or embodiment described herein can be the native coding sequence or a degenerate sequence, such as a codon-optimized coding sequence designed for a particular host cell.

The aminotransferase coding sequence, or a subsequence thereof, (e.g., the subsequence encoding the (A,C)-Xn-(GSLS)-Xn-SKA(L/l)(L/l) motif) as well as the aminotransferase polypeptide, or a fragment thereof (e.g., the (A,C)-Xn-(GSLS)-Xn-SKA(L/l)(L/l) motif), may be used to design nucleic acid probes to identify and clone DNA encoding a parent from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin).

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with the aminotransferase coding sequence, or a subsequence thereof, the carrier material is used in a Southern blot.

In one aspect, the nucleic acid probe is a polynucleotide comprising SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251; or a subsequence thereof. In another aspect, the nucleic acid probe is a polynucleotide that encodes the polypeptide of SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234; or a fragment thereof.

For purposes of the probes described above, hybridization indicates that the polynucleotide hybridizes to a labeled nucleic acid probe, or the full-length complementary strand thereof, or a subsequence of the foregoing; under very low to very high stringency conditions. Molecules to which the nucleic acid probe hybridizes under these conditions can be detected using, for example, X-ray film. Stringency and washing conditions are defined as described supra.

In one embodiment, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 239. (Sambrook et al., 1989, Molecular Cloning, A Laboratory Manual, 2d edition, Cold Spring Harbor, N.Y.).

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 240.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 241.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 242.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 243.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 244.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 245.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 246.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 247.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 248.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 249.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 250.

In another aspect, the aminotransferase (e.g., BAAT) is encoded by a polynucleotide that hybridizes under at least low stringency conditions, e.g., medium stringency conditions, medium-high stringency conditions, high stringency conditions, or very high stringency conditions with the full-length complementary strand of SEQ ID NO: 251.

The aminotransferase (e.g., BAAT) may be obtained from microorganisms of any suitable genus, including those readily available within the UniProtKB database (www.uniprot.org).

The aminotransferase (e.g., BAAT comprising the (A,C)-Xn-(GSLS)-Xn-SKA(L/l)(L/l) motif) may be a bacterial aminotransferase. For example, the aminotransferase may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces aminotransferase, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma aminotransferase.

In one embodiment, the aminotransferase (e.g., BAAT comprising the (A,C)-Xn-(GSLS)-Xn-SKA(L/l)(L/l) motif) is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis aminotransferase.

In another embodiment, the aminotransferase (e.g., BAAT comprising the (A,C)-Xn-(GSLS)-Xn-SKA(L/l)(L/l) motif) is a Streptococcus equisimilis, Streptococcus pyogenes, Streptococcus uberis, or Streptococcus equi subsp. Zooepidemicus aminotransferase.

In another embodiment, the aminotransferase (e.g., BAAT comprising the (A,C)-Xn-(GSLS)-Xn-SKA(L/l)(L/l) motif) is a Streptomyces achromogenes, Streptomyces avermitilis, Streptomyces coelicolor, Streptomyces griseus, or Streptomyces lividans aminotransferase.

The aminotransferase (e.g., BAAT comprising the (A,C)-Xn-(GSLS)-Xn-SKA(L/l)(L/l) motif) may be a fungal aminotransferase. For example, the aminotransferase may be a yeast aminotransferase such as a Candida, Kluyveromyces, Pichia, Saccharomyces, Schizosaccharomyces, Yarrowia or Issatchenkia aminotransferase or a filamentous fungal aminotransferase such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria aminotransferase.

In another embodiment, the aminotransferase (e.g., BAAT comprising the (A,C)-Xn-(GSLS)-Xn-SKA(L/l)(L/l) motif) is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis aminotransferase

In another embodiment, the aminotransferase (e.g., BAAT comprising the (A,C)-Xn-(GSLS)-Xn-SKA(L/l)(L/l) motif) is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicolalanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride aminotransferase.

In one aspect, the aminotransferase (e.g., BAAT comprising the (A,C)-Xn-(GSLS)-Xn-SKA(L/l)(L/l) motif) is from Pichia, such as the Pichia membranifaciens aminotransferase of SEQ ID NO: 206, or the Pichia farinosa aminotransferase of SEQ ID NO: 234.

In another aspect, the aminotransferase (e.g., BAAT comprising the (A,C)-Xn-(GSLS)-Xn-SKA(L/l)(L/l) motif) is from Candida, such as the Candida arabinofermentans aminotransferase of SEQ ID NO: 207, the Candida guilliermondii aminotransferase of SEQ ID NO: 209, the Candida parapsilosis aminotransferase of SEQ ID NO: 218, the Candida lusitaniae aminotransferase of SEQ ID NO: 224, the Candida albicans aminotransferase of SEQ ID NO: 226, or the Candida tropicalis aminotransferase of SEQ ID NO: 230.

In another aspect, the aminotransferase (e.g., BAAT comprising the (A,C)-Xn-(GSLS)-Xn-SKA(L/l)(L/l) motif) is from Brettanomyces, such as the Brettanomyces bruxellensis aminotransferase of SEQ ID NO: 208.

In another aspect, the aminotransferase (e.g., BAAT comprising the (A,C)-Xn-(GSLS)-Xn-SKA(L/l)(L/l) motif) is from Scheffersomyces, such as the Scheffersomyces stipitis aminotransferase of SEQ ID NO: 220.

In another aspect, the aminotransferase (e.g., BAAT comprising the (A,C)-Xn-(GSLS)-Xn-SKA(L/l)(L/l) motif) is from Lodderomyces, such as the Lodderomyces elongisporus aminotransferase of SEQ ID NO: 222.

In another aspect, the aminotransferase (e.g., BAAT comprising the (A,C)-Xn-(GSLS)-Xn-SKA(L/l)(L/l) motif) is from Debaryomyces, such as the Debaryomyces hansenii aminotransferase of SEQ ID NO: 228.

In another aspect, the aminotransferase (e.g., BAAT comprising the (A,C)-Xn-(GSLS)-Xn-SKA(L/l)(L/l) motif) is from, such as the Kuraishia capsulata aminotransferase of SEQ ID NO: 232.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The aminotransferases (e.g., BAAT comprising the (A,C)-Xn-(GSLS)-Xn-SKA(L/l)(L/l) motif) may also be identified and obtained from other sources including microorganisms isolated from nature (e.g., soil, composts, water, silage, etc.) or DNA samples obtained directly from natural materials (e.g., soil, composts, water, silage, etc.) using the above-mentioned probes. Techniques for isolating microorganisms and DNA directly from natural habitats are well known in the art. The polynucleotide encoding a aminotransferase may then be derived by similarly screening a genomic or cDNA library of another microorganism or mixed DNA sample.

Once a polynucleotide encoding a aminotransferase (e.g., BAAT comprising the (A,C)-Xn-(GSLS)-Xn-SKA(L/l)(L/l) motif) has been detected with a suitable probe as described herein, the sequence may be isolated or cloned by utilizing techniques that are known to those of ordinary skill in the art (see, e.g., Sambrook et al., 1989, supra). Techniques used to isolate or clone polynucleotides encoding aminotransferases include isolation from genomic DNA, preparation from cDNA, or a combination thereof. The cloning of the polynucleotides from such genomic DNA can be effected, e.g., by using the well-known polymerase chain reaction (PCR) or antibody screening of expression libraries to detect cloned DNA fragments with shares structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleotide sequence-based amplification (NASBA) may be used.

The aminotransferase (e.g., BAAT comprising the (A,C)-Xn-(GSLS)-Xn-SKA(L/l)(L/l) motif) may be a fused polypeptide or cleavable fusion polypeptide in which another polypeptide is fused at the N-terminus or the C-terminus of the aminotransferase. A fused polypeptide may be produced by fusing a polynucleotide encoding another polypeptide to a polynucleotide encoding the aminotransferase. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

### Hosts, Expression Vectors and Nucleic Acid Constructs

The recombinant cells described herein may be selected from any host cell capable of having an active 3-HP pathway. Those skilled in the art will understand that the genetic alterations, including metabolic modifications exemplified herein, may be described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art can apply the teachings and guidance provided herein to other organisms. For example, the metabolic alterations exemplified herein can readily be applied to other species by incorporating the same or analogous encoding nucleic acid from species other than the referenced species. In some embodiments, the host cells are isolated.

The recombinant host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell, including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397), or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804) or conjugation (see, e.g., Clewell, 1981, Microbiol. Rev. 45: 409-436). However, any method known in the art for introducing DNA into a host cell can be used.

The recombinant host cell may also be a eukaryote, such as a mammalian, insect, plant, or fungal cell.

“Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

In one embodiment, the host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes described herein, yeast shall be defined as described in Biology and Activities of Yeast (Skinner, F. A., Passmore, S. M., and Davenport, R. R., eds, Soc. App. Bacteriol. Symposium Series No. 9, 1980).

The yeast host cell may be a Candida, Hansenula, Issatchenkia, Kluyveromyces, Pichia, Saccharomyces, Schizosaccharomyces, or Yarrowia cell, such as a Candida sonorensis, Candida methanosorbosa, Candida ethanolica, Issatchenkia orientalis, Kluyveromyces lactis, Kluyveromyces marxianus, Pichia fermentans, Pichia galeiformis, Pichia membranifaciens, Pichia deserticola, Saccharomyces carlsbergensis, Saccharomyces cerevisiae, Saccharomyces diastaticus, Saccharomyces bulderi, Saccharomyces douglasii, Saccharomyces kluyveri, Saccharomyces norbensis, Saccharomyces oviformis, or Yarrowia lipolytica cell.

The yeast host cell may be a crabtree-positive phenotype or a crabtree-negative phenotype. Crabtree-negative organisms are characterized by the ability to be induced into an increased fermentative state. Both naturally occurring organisms and recombinant organisms can be characterized as Crabtree-negative. The Crabtree effect is defined as oxygen consumption inhibition in a microorganism when the microorganism is cultured under aerobic conditions in the presence of a high concentration of glucose (e.g. >5 mM glucose). Crabtree-positive organisms continue to ferment (rather than respire) irrespective of oxygen availability in the presence of glucose, while Crabtree-negative organisms do not exhibit glucose-mediated inhibition of oxygen consumption. This characteristic is useful for organic product synthesis, since it permits cells to be grown at high substrate concentrations but to retain the beneficial energetic effects of oxidative phosphorylation. In one aspect, the yeast has a crabtree-negative phenotype.

In certain embodiments the host yeast cell belongs to the genus Issatchenkia, Candida, or Saccharomyces, and in certain of these embodiments the host cell belongs to the Saccharomyces clade or the I. orientalis/P. fermentans clade. In certain of embodiments, the host cell is C. lambica, I. orientalis, or S. bulderi. In certain embodiments, the yeast cells are CNB1 yeast cells (as described in WO2012/074818, the content of which is incorporated herein by reference) and/or CB1 yeast cells. CB1 cells include CNB1 cells described in WO2012/074818 and any cell related to or derived from the CNB1 cells described therein.

The yeast host cell may be derived from a cell or engineered such that the cell has been genetically modified to produce high lactate or 3-HP titers, exhibit increased tolerance to acidic pH, exhibit increased tolerance to a fermentation product (e.g., lactate, 3-HP, ethanol or propanol), and/or display increased ability to ferment pentose sugars (yet, in some embodiments, the yeast cell is unable to ferment pentose sugars). Exemplary genetically modified yeast cells are described in WO00/71738, WO03/049525, WO03/102201, WO03/102152, WO02/42471, WO2007/032792, WO2007/106524, WO2007/117282, the content of which is hereby incorporated by reference with respect to said cells. The modification of any yeast cell described in the foregoing applications is contemplated with an active 3-HP pathway as described herein.

The host cell may have improved resistance to 3-HP. In certain embodiments, the host cells are “3-HP resistant yeast cells,” as described in US2012/0135481. In certain of these embodiments, the host cells may exhibit 3-HP resistance in their native form. In other embodiments, the cells may have undergone mutation and/or selection (e.g., chemostat selection or repeated serial subculturing) before, during, or after introduction of genetic modifications related to an active 3-HP pathway, such that the mutated and/or selected cells possess a higher degree of resistant to 3-HP than wild-type cells of the same species. For example, in some embodiments, the cells have undergone mutation and/or selection in the presence of 3-HP or lactic acid before or after being genetically modified with one or more heterologous 3-HP pathway genes. In certain embodiments, mutation and/or selection may be carried out on cells that exhibit 3-HP resistance in their native form. Cells that have undergone mutation and/or selection may be tested for sugar consumption and other characteristics in the presence of varying levels of 3-HP in order to determine their potential as industrial hosts for 3-HP production. In addition to 3-HP resistance, the host cells may have undergone mutation and/or selection for resistance to one or more additional organic acids (e.g., lactic acid) or to other fermentation products, byproducts, or media components. Selection, such as selection for resistance to 3-HP or to other compounds, may be accomplished using methods well known in the art (e.g., as described in US2012/0135481).

In one embodiment, the host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920, or as described herein.

The ideal cell for organic acids, such 3-HP, is capable of growing at low pH levels. The ability to conduct fermentation at a low pH decreases downstream recovery costs, resulting in more economical production. Therefore, in certain embodiments the host cell is capable of growing at low pH levels (e.g., at pH levels less than 7, 6, 5, 4, or 3).

A suitable host cell may possess one or more favorable characteristics in addition to organic acid resistance and/or low pH growth capability. For example, potential host cells exhibiting acid resistance may be further selected based on glycolytic rates, specific growth rates, thermotolerance, tolerance to biomass hydrolysate inhibitors, overall process robustness, and so on. These criteria may be evaluated prior to any genetic modification relating to a metabolic pathway, or they may be evaluated after one or more such modifications have taken place.

In some aspects, the recombinant cell comprises one or more (e.g., two, several) heterologous polynucleotides of an active 3-HP pathway described herein (e.g., a heterologous polynucleotide encoding a PPC; a heterologous polynucleotide encoding a PYC; a heterologous polynucleotide encoding an AAT; a heterologous polynucleotide encoding an ADC; a heterologous polynucleotide encoding a BAAT or gabT; and/or a heterologous polynucleotide encoding a 3-HPDH), wherein the recombinant cell secretes (and/or is capable of secreting) an increased level of 3-HP compared to the host cell without the one or more heterologous polynucleotides of the active 3-HP pathway when cultivated under the same conditions. In some aspects (or the methods described herein), the recombinant cell secretes and/or is capable of secreting an increased level of 3-HP of at least 5%, e.g., at least 10%, at least 15%, at least 20%, at least 25%, at least 50%, at least 100%, at least 150%, at least 200%, at least 300%, or at 500% compared to the host cell without the one or more heterologous polynucleotides of the active 3-HP pathway, when cultivated under the same conditions. Examples of suitable cultivation conditions are described below and will be readily apparent to one of skill in the art based on the teachings herein.

In any of these aspects (or the methods described herein), the recombinant cell produces (and/or is capable of producing) a fermentation product, such 3-HP, at a yield of at least 10%, e.g., at least 20%, at least 30%, at least 40%, at least 50%, at least 60%, at least 70%, at least 80%, or at least 90%, of theoretical.

In any of these aspects (or the methods described herein), the recombinant cell may have a 3-HP volumetric productivity greater than about 0.1 g/L per hour, e.g., greater than about 0.2 g/L per hour, 0.5 g/L per hour, 0.6 g/L per hour, 0.7 g/L per hour, 0.8 g/L per hour, 0.9 g/L per hour, 1.0 g/L per hour, 1.1 g/L per hour, 1.2 g/L per hour, 1.3 g/L per hour, 1.5 g/L per hour, 1.75 g/L per hour, 2.0 g/L per hour, 2.25 g/L per hour, 2.5 g/L per hour, 2.75 g/L per hour, 3.0 g/L per hour, 3.25 g/L per hour, 3.5 g/L per hour, 3.75 g/L per hour, or 4.0 g/L per hour; or between about 0.1 g/L per hour and about 2.0 g/L per hour, e.g., between about 0.3 g/L per hour and about 1.7 g/L per hour, about 0.5 g/L per hour and about 1.5 g/L per hour, about 0.7 g/L per hour and about 1.3 g/L per hour, about 0.8 g/L per hour and about 1.2 g/L per hour, or about 0.9 g/L per hour and about 1.1 g/L per hour; or between about 0.1 g/L per hour and about 4.0 g/L per hour, e.g., between about 0.5 g/L per hour and about 3.75 g/L per hour, about 1.0 g/L per hour and about 3.5 g/L per hour, about 2.0 g/L per hour and about 3.25 g/L per hour.

In any of these aspects, the recombinant cell produces (and/or is capable of producing) a greater amount of 3-HP compared to the cell without the heterologous polynucleotide encoding the aminotransferase (e.g., BAAT comprising the (A,C)-Xn-(GSLS)-Xn-SKA(L/l)(L/l) motif) when cultivated under the same conditions. In some embodiments, the cell produces (and/or is capable of producing) at least 10% more (e.g., at least 15%, at least 20%, at least 25%, at least 30%, at least 35%, at least 40%, at least 45%, at least 50%, at least 75%, at least 100%, at least 200%, at least 300%, or at 500% more) 3-HP compared to the cell without the heterologous polynucleotide encoding the aminotransterase when cultivated under the same conditions.

In any of these aspects, the recombinant cell produces (and/or is capable of producing) a greater amount of 3-HP (e.g., at least 10% more, at least 15%, at least 20%, at least 25%, at least 30%, at least 35%, at least 40%, at least 45%, at least 50%, at least 75%, at least 100%, at least 200%, at least 300%, or at 500% more) compared to a second recombinant cell, when cultivated under identical conditions; wherein the second cell is identical to the recombinant cell with the proviso that the second cell encodes the BAAT of SEQ ID NO: 20 in place of the recombinant cell aminotransferase.

In some embodiments, the recombinant cells have an increased level of aminotransferase activity (e.g., BAAT activity) compared to the host cells without the heterologous polynucleotide encoding the aminotransferase, when cultivated under the same conditions. In other aspects, the cells have an increased level of aminotransferase activity of at least 5%, e.g., at least 10%, at least 15%, at least 20%, at least 25%, at least 50%, at least 100%, at least 150%, at least 200%, at least 300%, or at 500% compared to the host cells without the heterologous polynucleotide encoding aminotransferase, when cultivated under the same conditions.

The recombinant cells may be cultivated in a suitable nutrient medium using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the desired polypeptide to be expressed and/or isolated. The cultivation takes place in a suitable nutrient medium comprising carbon and nitrogen sources and inorganic salts, as described herein, using procedures known in the art. Suitable media are available from commercial suppliers, may be prepared according to published compositions (e.g., in catalogues of the American Type Culture Collection), or may be prepared from commercially available ingredients.

The recombinant cells described herein also can be subjected to adaptive evolution to further augment product biosynthesis, including under conditions approaching theoretical maximum growth.

The recombinant cells described herein may utilize expression vectors comprising the coding sequence of one or more (e.g., two, several) heterologous 3-HP pathway genes linked to one or more control sequences that direct expression in a suitable cell under conditions compatible with the control sequence(s). Such expression vectors may be used in any of the cells and methods described herein. The polynucleotides described herein may be manipulated in a variety of ways to provide for expression of a desired polypeptide. Manipulation of the polynucleotide prior to its insertion into a vector may be desirable or necessary depending on the expression vector. The techniques for modifying polynucleotides utilizing recombinant DNA methods are well known in the art.

A construct or vector (or multiple constructs or vectors) comprising the one or more (e.g., two, several) heterologous 3-HP pathway genes may be introduced into a cell so that the construct or vector is maintained as a chromosomal integrant or as a self-replicating extra-chromosomal vector as described earlier.

The various nucleotide and control sequences may be joined together to produce a recombinant expression vector that may include one or more (e.g., two, several) convenient restriction sites to allow for insertion or substitution of the polynucleotide at such sites. Alternatively, the polynucleotide(s) may be expressed by inserting the polynucleotide(s) or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

In one aspect, the recombinant cell comprises a heterologous polynucleotide is contained in an independent vector. In one aspect, the recombinant cell comprises a plurality of heterologous polynucleotides each contained in an independent vector. In one aspect, the recombinant cell comprises at least two heterologous polynucleotides contained in a single vector. In one aspect, the recombinant cell comprises at least three of the heterologous polynucleotides contained on a single vector. In one aspect, the recombinant cell comprises at least four of the heterologous polynucleotides contained on a single vector. In one aspect, all the heterologous polynucleotides of the recombinant cell are contained on a single vector. Polynucleotides encoding heteromeric subunits of a protein complex may be contained in a single heterologous polynucleotide on a single vector or alternatively contained in separate heterologous polynucleotides on separate vectors.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the cell, or a transposon, may be used.

The expression vector may contain any suitable promoter sequence that is recognized by a cell for expression of a pyruvate reductase gene or any 3-HP pathway gene described herein. The promoter sequence contains transcriptional control sequences that mediate the expression of the polypeptide. The promoter may be any polynucleotide that shows transcriptional activity in the cell of choice including mutant, truncated, and hybrid promoters, and may be obtained from genes encoding extracellular or intracellular polypeptides either homologous or heterologous to the cell.

Each heterologous polynucleotide described herein may be operably linked to a promoter that is foreign to the polynucleotide. For example, in one aspect, the heterologous polynucleotide encoding the aminotransferase (e.g., BAAT comprising the (A,C)-Xn-(GSLS)-Xn-SKA(L/l)(L/l) motif) is operably linked to a promoter foreign to the polynucleotide. In another aspect, the heterologous polynucleotide encoding a polypeptide of a 3-HP pathway described herein (e.g., one or more of a PPC, PYC, AAT, ADC, BAAT, gabT, or 3-HPDH) is operably linked to promoter foreign to the polynucleotide. The promoters may be identical to or share a high degree of sequence identity (e.g., at least about 80%, at least about 85%, at least about 90%, at least about 95%, or at least about 99%) with a selected native promoter.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing the transcription of the nucleic acid constructs in a yeast cells, include, but are not limited to, the promoters obtained from the genes for enolase, (e.g., S. cerevisiae enolase or I. orientalis enolase (ENO1)), galactokinase (e.g., S. cerevisiae galactokinase or I. orientalis galactokinase (GAL1)), alcohol dehydrogenase/glyceraldehyde-3-phosphate dehydrogenase (e.g., S. cerevisiae alcohol dehydrogenase/glyceraldehyde-3-phosphate dehydrogenase or I. orientalis alcohol dehydrogenase/glyceraldehyde-3-phosphate dehydrogenase (ADH1, ADH2/GAP)), triose phosphate isomerase (e.g., S. cerevisiae triose phosphate isomerase or I. orientalis triose phosphate isomerase (TPI)), metallothionein (e.g., S. cerevisiae metallothionein or I. orientalis metallothionein (CUP1)), 3-phosphoglycerate kinase (e.g., S. cerevisiae 3-phosphoglycerate kinase or I. orientalis 3-phosphoglycerate kinase (PGK)), PDC1, xylose reductase (XR), xylitol dehydrogenase (XDH), L-(+)-lactate-cytochrome c oxidoreductase (CYB2), translation elongation factor-1 (TEF1), translation elongation factor-2 (TEF2), glyceraldehyde-3-phosphate dehydrogenase (GAPDH), and orotidine 5′-phosphate decarboxylase (URA3) genes. Other useful promoters for yeast host cells are described by Romanos et al., 1992, Yeast 8: 423-488.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dania (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

The control sequence may also be a suitable transcription terminator sequence, which is recognized by a host cell to terminate transcription. The terminator sequence is operably linked to the 3′-terminus of the polynucleotide encoding the polypeptide. Any terminator that is functional in the yeast cell of choice may be used. The terminator may be identical to or share a high degree of sequence identity (e.g., at least about 80%, at least about 85%, at least about 90%, at least about 95%, or at least about 99%) with the selected native terminator. In certain embodiments, 3-HP pathway genes are linked to a terminator that comprises a functional portion of a native GAL10 gene native to the host cell or a sequence that shares at least 80%, at least 85%, at least 90%, or at least 95% sequence identity with a native GAL10 terminator.

Suitable terminators for bacterial host cells may be obtained from the genes for Bacillus clausii alkaline protease (aprH), Bacillus licheniformis alpha-amylase (amyL), and Escherichia coli ribosomal RNA (rrnB).

Suitable terminators for yeast host cells may be obtained from the genes for enolase (e.g., S. cerevisiae or I. orientalis enolase cytochrome C (e.g., S. cerevisiae or I. orientalis cytochrome (CYC1)), glyceraldehyde-3-phosphate dehydrogenase (e.g., S. cerevisiae or I. orientalis glyceraldehyde-3-phosphate dehydrogenase (gpd)), PDC1, XR, XDH, transaldolase (TAL), transketolase (TKL), ribose 5-phosphate ketol-isomerase (RKI), CYB2, and the galactose family of genes (especially the GAL10 terminator). Other useful terminators for yeast host cells are described by Romanos et al., 1992, supra.

Suitable terminators for filamentous fungal host cells may be obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a suitable leader sequence, when transcribed is a nontranslated region of an mRNA that is important for translation by the host cell. The leader sequence is operably linked to the 5′-terminus of the polynucleotide encoding the polypeptide. Any leader sequence that is functional in the yeast cell of choice may be used.

Suitable leaders for yeast host cells are obtained from the genes for enolase (e.g., S. cerevisiae or I. orientalis enolase (ENO-1)), 3-phosphoglycerate kinase (e.g., S. cerevisiae or I. orientalis 3-phosphoglycerate kinase), alpha-factor (e.g., S. cerevisiae or I. orientalis alpha-factor), and alcohol dehydrogenase/glyceraldehyde-3-phosphate dehydrogenase (e.g., S. cerevisiae or I. orientalis alcohol dehydrogenase/glyceraldehyde-3-phosphate dehydrogenase (ADH2/GAP)).

Preferred leaders for filamentous fungal host cells are obtained from the genes for Aspergillus oryzae TAKA amylase and Aspergillus nidulans triose phosphate isomerase.

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell of choice may be used. Useful polyadenylation sequences for yeast cells are described by Guo and Sherman, 1995, Mol. Cellular Biol. 15: 5983-5990. Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

The vectors may contain one or more (e.g., two, several) selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vectors may contain one or more (e.g., two, several) elements that permit integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. Potential integration loci include those described in the art (e.g., See US2012/0135481).

For autonomous replication, the vector may further comprise an origin of replication enabling the vector to replicate autonomously in the yeast cell. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide described herein may be inserted into a host cell to increase production of a polypeptide. An increase in the copy number of the polynucleotide can be obtained by integrating at least one additional copy of the sequence into the yeast cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors described herein are well known to one skilled in the art (see, e.g., Sambrook et al., 1989, supra).

Additional procedures and techniques known in the art for the preparation of recombinant cells comprising one or more 3-HP pathway genes, are described in, e.g., WO 2012/074818, the content of which is hereby incorporated by reference.

### Active 3-HP Pathway

The host cells described herein may comprise an active 3-HP pathway. 3-HP pathways, 3-HP pathway genes and corresponding engineered transformants for fermentation of 3-HP are known in the art (e.g., US Publication No. 2012/0135481; U.S. Pat. No. 6,852,517; U.S. Pat. No. 7,309,597; US Pub. No. 2001/0021978; US Pub. No. 2008/0199926; WO02/42418; and WO10/031083; the content of which is hereby incorporated in its entirety). An overview of several known 3-HP pathways is shown in FIG. 1.

In certain embodiments, the recombinant cells provided herein have an active 3-HP pathway that proceeds through a β-alanine and/or malonate semialdehyde intermediate. Several 3-HP pathways are known in the art to proceed through a malonate semialdehyde intermediate. For example, the cells may have an active 3-HP pathway that proceeds is through PEP or pyruvate, OAA, aspartate, β-alanine, and malonate semialdehyde intermediates (see, e.g., U.S. Pat. No. 7,186,541; FIG. 55). In these embodiments, the recombinant cells comprise a set of 3-HP pathway genes comprising one or more of pyruvate carboxylase (PYC), PEP carboxylase (PPC), aspartate aminotransferase (AAT), aspartate 1-decarboxylase (ADC), β-alanine aminotransferase (BAAT), aminobutyrate aminotransferase (gabT), 3-HP dehydrogenase (3-HPDH), 3-hydroxyisobutyrate dehydrogenase (HIBADH), and 4-hydroxybutyrate dehydrogenase genes. The 3-HP pathway genes may also include a PEP carboxykinase (PCK) gene that has been modified to produce a polypeptide that preferably catalyzes the conversion of PEP to OAA (native PCK genes generally produce a polypeptide that preferably catalyzes the reverse reaction of OAA to PEP).

In another example, the recombinant cells may have an active 3-HP pathway that proceeds through PEP or pyruvate, OAA, and malonate semialdehyde intermediates (see, e.g., US Pub. No. 2010/0021978, FIG. 1). In these embodiments, the cells comprise a set of 3-HP pathway genes comprising one or more of PPC, PYC, 2-keto acid decarboxylase, alpha-ketoglutarate (AKG) decarboxylase (KG D), branched-chain alpha-keto acid decarboxylase (BCKA), indolepyruvate decarboxylase (IPDA), 3-HPDH, HIBADH, and 4-hydroxybutyrate dehydrogenase genes. The 3-HP pathway genes may also include a PCK gene that has been modified to produce a polypeptide that preferably catalyzes the conversion of PEP to OAA. Further, the 3-HP pathway genes may include a PDC gene and/or benzoylformate decarboxylase gene that has been modified to encode a polypeptide capable of catalyzing the conversion of OAA to malonate semialdehyde.

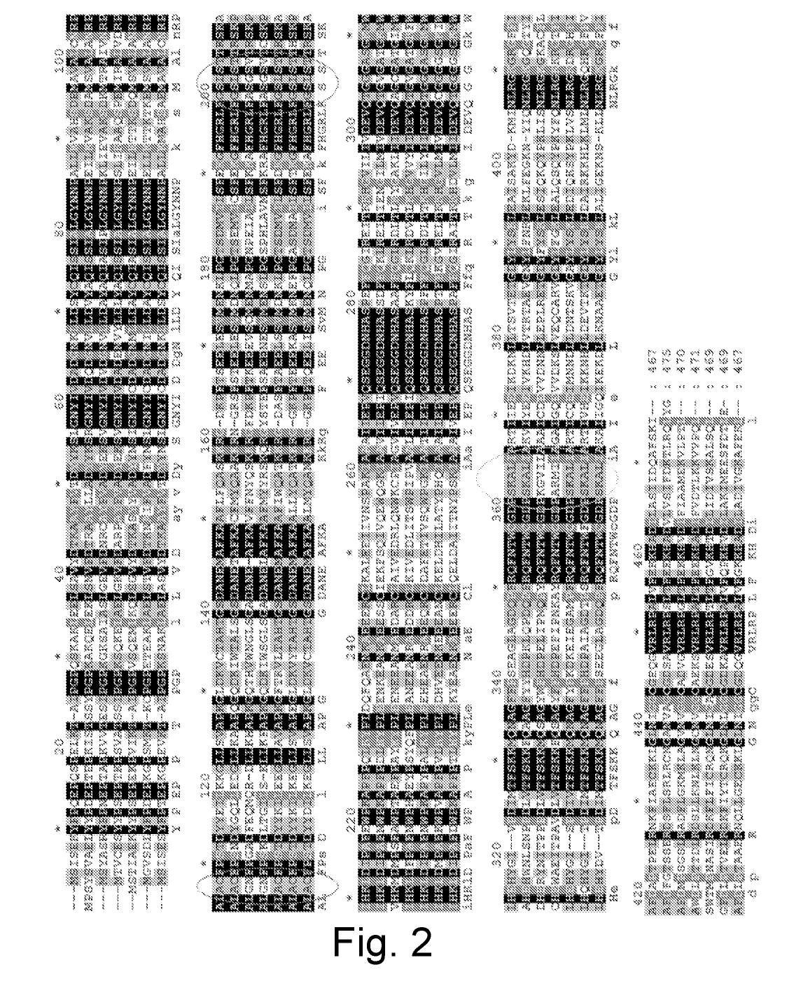

In another example, the recombinant cells may have an active 3-HP pathway that proceeds through PEP or pyruvate, OAA, malonyl-CoA, and malonate semialdehyde intermediates, wherein the malonate semialdehyde intermediate is optional (see, e.g., US Pub. No. 2010/0021978, FIG. 2). In these embodiments, the cells comprise a set of 3-HP pathway genes comprising one or more of PPC, PYC, OAA formatelyase, malonyl-CoA reductase, CoA acylating malonate semialdehyde dehydrogenase, 3-HPDH, HIBADH, and 4-hydroxybutyrate dehydrogenase genes. The 3-HP pathway genes may also include a PCK gene that has been modified to produce a polypeptide that preferably catalyzes the conversion of PEP to OAA. Further, the 3-HP pathway genes may include an OAA dehydrogenase gene derived by modifying a 2-keto-acid dehydrogenase gene to produce a polypeptide that catalyzes the conversion of OAA to malonyl-CoA.

In another example, the recombinant cells may have an active 3-HP pathway that proceeds through pyruvate, acetyl-CoA, malonyl-CoA, and malonate semialdehyde intermediates, wherein the malonate semialdehyde intermediate is optional (see, e.g., U.S. Pat. No. 7,186,541; FIG. 44). In these embodiments, the cells comprise a set of 3-HP pathway genes comprising one or more of pyruvate dehydrogenase (PDH), acetyl-CoA carboxylase (ACC), malonyl-CoA reductase, CoA acylating malonate semialdehyde dehydrogenase, 3-HPDH, HIBADH, and 4-hydroxybutyrate dehydrogenase genes.

In another example, the recombinant cells may have an active 3-HP pathway that proceeds through pyruvate, alanine, β-alanine, β-alanyl-CoA, acrylyl-CoA, 3-HP-CoA, and malonate semialdehyde intermediates, wherein the β-alanyl-CoA, acrylyl-CoA, 3-HP-CoA, and malonate semialdehyde intermediates are optional (β-alanine can be converted to 3-HP via a malonate semialdehyde intermediate or via β-alanyl-CoA, acrylyl-CoA, and 3-HP-CoA intermediates (see, e.g., U.S. Pat. No. 7,309,597, FIG. 1). In these embodiments, the cells comprise a set of 3-HP pathway genes comprising one or more of alanine dehydrogenase, pyruvate/alanine aminotransferase, alanine 2,3 aminomutase, CoA transferase, CoA synthetase, β-alanyl-CoA ammonia lyase, 3-HP-CoA dehydratase, 3-HP-CoA hydrolase, 3-hydroxyisobutyryl-CoA hydrolase, BAAT, 3-HPDH, HIBADH, and 4-hydroxybutyrate dehydrogenase genes.

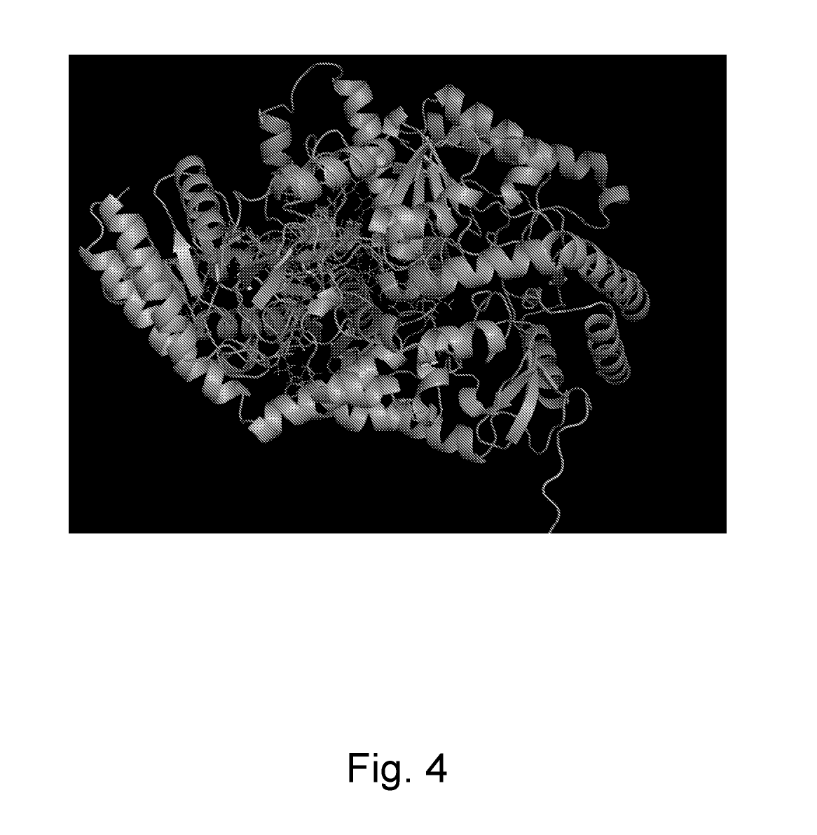

Other pathways that do not utilize a malonate semialdehyde intermediate are also known in the art. For example, the recombinant cells may have an active 3-HP pathway that proceeds through PEP or pyruvate, OAA, and malate intermediates (see, e.g., US Pub. No. 2010/0021978; FIG. 4). In these embodiments, the cells comprise a set of 3-HP pathway genes comprising one or more of PPC, PYC, malate dehydrogenase, and malate decarboxylase genes. The 3-HP pathway genes may also include a PCK gene that has been modified to produce a polypeptide that preferably catalyzes the conversion of PEP to OAA.

In another example, the recombinant cells may have an active 3-HP pathway that proceeds through pyruvate, lactate, lactyl-CoA, acrylyl-CoA, and 3-HP-CoA intermediates (see, e.g., WO02/042418, FIG. 1). In these embodiments, the cells comprise a set of 3-HP pathway genes comprising one or more of LDH, CoA transferase, CoA synthetase, lactyl-CoA dehydratase, 3-HP-CoA dehydratase, 3-HP-CoA hydrolase, and 3-hydroxyisobutyryl-CoA hydrolase genes.

In another example, the recombinant cells may have an active 3-HP pathway that proceeds through glycerol and 3-HPA intermediates (see, e.g., U.S. Pat. No. 6,852,517). In these embodiments, the cells comprise a set of 3-HP pathway genes comprising one or more of glycerol dehydratase and aldehyde dehydrogenase genes.

In another example, the recombinant cells may have an active 3-HP pathway that proceeds through PEP or pyruvate, OAA, aspartate, β-alanine, β-alanyl-CoA, acrylyl-CoA, 3-HP-CoA, and alanine intermediates, wherein the OAA, aspartate, and alanine intermediates are optional (PEP or pyruvate can be converted to β-alanine via OAA and aspartate or via alanine) (see WO02/042418, FIG. 54; U.S. Pat. No. 7,309,597, FIG. 1). In these embodiments, the cells comprise a set of 3-HP pathway genes comprising one or more of PPC, PYC, AAT, ADC, CoA transferase, CoA synthetase, β-alanyl-CoA ammonia lyase, 3-HP-CoA dehydratase, 3-HP-CoA hydrolase, 3-hydroxyisobutyrl-CoA hydrolase, alanine dehydrogenase, pyruvate/alanine aminotransferase, and AAM genes. The 3-HP pathway genes may also include a PCK gene that has been modified to produce a polypeptide that preferably catalyzes the conversion of PEP to OAA.

In certain embodiments, the recombinant cells provided herein express one or more 3-HP pathway genes encoding enzymes selected from the group consisting of ACC (catalyzes the conversion of acetyl-CoA to malonyl-CoA), alanine 2,3 aminomutase (AAM, catalyzes the conversion of alanine to β-alanine), alanine dehydrogenase (catalyzes the conversion of pyruvate to alanine), aldehyde dehydrogenase (catalyzes the conversion of 3-HPA to 3-HP), KGD (catalyzes the conversion of OAA to malonate semialdehyde), AAT (catalyzes the conversion of OAA to aspartate), ADC (catalyzes the conversion of aspartate to β-alanine), BCKA (catalyzes the conversion of OAA to malonate semialdehyde), BAAT (catalyzes the conversion of β-alanine to malonate semialdehyde), 4-aminobutyrate aminotransferase (gabT, catalyzes the conversion of β-alanine to malonate semialdehyde), β-alanyl-CoA ammonia lyase (catalyzes the conversion of β-alanyl-CoA to acrylyl-CoA), Co-A acylating malonate semialdehyde dehydrogenase (catalyzes the conversion of malonyl-CoA to malonate semialdehyde), CoA synthetase (catalyzes the conversion of β-alanine to β-alanyl-CoA or the conversion of lactate to lactyl-CoA), CoA transferase (catalyzes the conversion of β-alanine to β-alanyl-CoA and/or the conversion of lactate to lactyl-CoA), glycerol dehydratase (catalyzes the conversion of glycerol to 3-HPA), IPDA (catalyzes the conversion of OAA to malonate semialdehyde), LDH (catalyzes the conversion of pyruvate to lactate), lactyl-CoA dehydratase (catalyzes the conversion of lactyl-CoA to acrylyl-CoA), malate decarboxylase (catalyzes the conversion of malate to 3-HP), malate dehydrogenase (catalyzes the conversion of OAA to malate), malonyl-CoA reductase (catalyzes the conversion of malonyl-CoA to malonate semialdehyde or 3-HP), OAA formatelyase (also known as pyruvate-formate lyase and ketoacid formate-lyase, catalyzes the conversion of OAA to malonyl-CoA), OAA dehydrogenase (catalyzes the conversion of OAA to malonyl CoA); PPC (catalyzes the conversion of PEP to OAA), pyruvate/alanine aminotransferase (catalyzes the conversion of pyruvate to alanine), PYC (catalyzes the conversion of pyruvate to OAA), PDH (catalyzes the conversion of pyruvate to acetyl-CoA), 2-keto acid decarboxylase (catalyzes the conversion of OAA to malonate semialdehyde), 3-HP-CoA dehydratase (also known as acrylyl-CoA hydratase, catalyzes the conversion of acrylyl-CoA to 3-HP-CoA), 3-HPDH (catalyzes the conversion of malonate semialdehyde to 3-HP), 3-HP-CoA hydrolase (catalyzes the conversion of 3-HP-CoA to 3-HP), HIBADH (catalyzes the conversion of malonate semialdehyde to 3-HP), 3-hydroxyisobutyryl-CoA hydrolase (catalyzes the conversion of 3-HP-CoA to 3-HP), and 4-hydroxybutyrate dehydrogenase (catalyzes the conversion of malonate semialdehyde to 3-HP). For each of these enzyme activities, the reaction of interest in parentheses may be a result of endogenous or heterologous activity.

Any suitable 3-HP pathway gene, endogenous or heterologous, may be used and expressed in sufficient amount to produce an enzyme involved in a selected active 3-HP pathway. With the complete genome sequence available for now more than 550 species (with more than half of these available on public databases such as the NCBI), including 395 microorganism genomes and a variety of yeast, fungi, plant, and mammalian genomes, the identification of genes encoding the selected 3-HP pathway enzymatic activities taught herein is routine and well known in the art for a selected host. For example, suitable homologues, orthologs, paralogs and nonorthologous gene displacements of known genes, and the interchange of genetic alterations between organisms can be identified in related or distant host to a selected host.

For recombinant cells without a known genome sequence, sequences for genes of interest (either as overexpression candidates or as insertion sites) can typically be obtained using techniques known in the art. Routine experimental design can be employed to test expression of various genes and activity of various enzymes, including genes and enzymes that function in a 3-HP pathway. Experiments may be conducted wherein each enzyme is expressed in the cell individually and in blocks of enzymes up to and including preferably all pathway enzymes, to establish which are needed (or desired) for improved 3-HP production. One illustrative experimental design tests expression of each individual enzyme as well as of each unique pair of enzymes, and further can test expression of all required enzymes, or each unique combination of enzymes. A number of approaches can be taken, as will be appreciated.

The recombinant host cells of the invention can be produced by introducing heterologous polynucleotides encoding one or more of the enzymes participating in a 3-HP pathway, as described below. As one in the art will appreciate, in some instances (e.g., depending on the selection of host) the heterologous expression of every gene shown in the 3-HP pathway may not be required for 3-HP production since a host cell may have endogenous enzymatic activity from one or more pathway genes. For example, if a chosen host is deficient in one or more enzymes of a 3-HP pathway, then heterologous polynucleotides for the deficient enzyme(s) are introduced into the host for subsequent expression. Alternatively, if the chosen host exhibits endogenous expression of some pathway genes, but is deficient in others, then an encoding polynucleotide is needed for the deficient enzyme(s) to achieve 3-HP biosynthesis. Thus, a recombinant host cell of the invention can be produced by introducing heterologous polynucleotides to obtain the enzyme activities of a desired biosynthetic pathway or a desired biosynthetic pathway can be obtained by introducing one or more heterologous polynucleotides that, together with one or more endogenous enzymes, produces a desired product such as 3-HP.

Depending on the 3-HP pathway constituents of a selected recombinant host organism, the host cells of the invention will include at least one heterologous polynucleotide and optionally up to all encoding heterologous polynucleotides for the 3-HP pathway. For example, 3-HP biosynthesis can be established in a host deficient in a 3-HP pathway enzyme through heterologous expression of the corresponding polynucleotide. In a host deficient in all enzymes of a 3-HP pathway, heterologous expression of all enzymes in the pathway can be included, although it is understood that all enzymes of a pathway can be expressed even if the host contains at least one of the pathway enzymes.

A “pyruvate carboxylase gene” or “PYC gene” as used herein refers to any gene that encodes a polypeptide with pyruvate carboxylase activity, meaning the ability to catalyze the conversion of pyruvate, CO₂, and ATP to OAA, ADP, and phosphate. In certain embodiments, the PYC gene may be derived from a bacterial source. For example, the PYC gene may be derived from one of the few bacterial species that use only PYC and not PPC (see below) for anaplerosis, such as R. sphaeroides, or from a bacterial species that possesses both PYC and PPC, such as R. etli. The amino acid sequences encoded by the PYC genes of R. sphaeroides and R. etli are set forth in SEQ ID NOs: 3 and 4, respectively. A PYC gene may be derived from a gene encoding the amino acid sequence of SEQ ID NOs: 3 or 4, or from a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NOs: 3 or 4. Alternatively, the PYC gene may be derived from a PYC gene encoding an enzyme that does not have a dependence on acetyl-CoA for activation, such as a P. fluorescens PYC gene encoding the amino acid sequence set forth in SEQ ID NO: 5 (carboxytransferase subunit) or SEQ ID NO: 6 (biotin carboxylase subunit), a C. glutamicum PYC gene of encoding the amino acid sequence set forth in SEQ ID NO: 7, or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NOs: 5, 6, or 7. A PYC gene may also be derived from a PYC gene that encodes an enzyme that is not inhibited by aspartate, such as an S. meliloti PYC gene encoding the amino acid sequence set forth in SEQ ID NO: 8 (Sauer FEMS Microbiol Rev 29:765 (2005), or from a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NO: 8. In certain embodiments, a PYC gene may be derived from a yeast source. For example, the PYC gene may be derived from an I. orientalis PYC gene encoding the amino acid sequence set forth in SEQ ID NO: 2. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 2. In certain embodiments, an I. orientalis-derived PYC gene may comprise the nucleotide sequence set forth in SEQ ID NO: 1 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 1.

A “PEP carboxylase gene” or “PPC gene” as used herein refers to any gene that encodes a polypeptide with PEP carboxylase activity, meaning the ability to catalyze the conversion of PEP and CO₂ to OAA and phosphate. In certain embodiments, a PPC gene may be derived from a bacterial PPC gene. For example, the PPC gene may be derived from an E. coli PPC gene encoding the amino acid sequence set forth in SEQ ID NO: 10 or an amino acid sequence with at least 50%, at least 60%, at least 70%, at least 80%, at least 85%, at least 90%, at least 95%, at least 97%, or at least 99% sequence identity to the amino acid sequence of SEQ ID NO: 10. In certain embodiments, an E. coli-derived PPC gene may comprise the nucleotide sequence set forth in SEQ ID NO: 9 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 9. In other embodiments, a PPC gene may be derived from an “A” type PPC, found in many archea and a limited number of bacteria, that is not activated by acetyl CoA and is less inhibited by aspartate. For example, a PPC gene may be derived from an M. thermoautotrophicum PPC A gene encoding the amino acid sequence set forth in SEQ ID NO: 11, a C. perfringens PPC A gene encoding the amino acid sequence set forth in SEQ ID NO: 12, or a gene encoding an amino acid sequence with at least 50%, at least 60%, at least 70%, at least 80%, at least 85%, at least 90%, at least 95%, at least 97%, or at least 99% sequence identity to the amino acid sequence of SEQ ID NOs: 11 or 12. In certain of these embodiments, the gene may have undergone one or more mutations versus the native gene in order to generate an enzyme with improved characteristics. For example, the gene may have been mutated to encode a PPC polypeptide with increased resistance to aspartate feedback versus the native polypeptide. In other embodiments, the PPC gene may be derived from a plant source.

An “aspartate aminotransferase gene” or “AAT gene” as used herein refers to any gene that encodes a polypeptide with aspartate aminotransferase activity, meaning the ability to catalyze the conversion of OAA to aspartate. Enzymes having aspartate aminotransferase activity are classified as EC 2.6.1.1. In certain embodiments, the AAT gene may be derived from a bacterial source. For example, the AAT gene may be derived from an E. coli aspC gene encoding a polypeptide comprising the amino acid sequence set forth in SEQ ID NO: 16. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 16. In certain embodiments, an AAT gene may be derived from a yeast source such as S. cerevisiae or I. orientalis. For example, the AAT gene may be derived from an I. orientalis AAT gene encoding the amino acid sequence set forth in SEQ ID NO: 14 or an S. cerevisiae AAT2 gene encoding the amino acid sequence set forth in SEQ ID NO: 15. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NOs: 14 or 15. In certain embodiments, an I. orientalis-derived AAT gene may comprise the nucleotide sequence set forth in SEQ ID NO: 13 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 13.

An “aspartate decarboxylase gene” or “ADC gene” as used herein refers to any gene that encodes a polypeptide with aspartate decarboxylase activity, meaning the ability to catalyze the conversion of aspartate to β-alanine. Enzymes having aspartate decarboxylase activity are classified as EC 4.1.1.11. In certain embodiments, an ADC gene may be derived from a bacterial source.

In some embodiments, the ADC gene may be derived from an S. avermitilis panD gene encoding the amino acid sequence set forth in SEQ ID NO: 17. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 17. In certain embodiments, an S. avermitilis-derived ADC gene may comprise the nucleotide sequence set forth in any one of SEQ ID NOs: 130, 145, 146, or 147; or a nucleotide sequence with at least 50%, at least 60%, at least 70%, at least 80%, at least 85%, at least 90%, at least 95%, at least 97%, or at least 99% sequence identity to the nucleotide sequence set forth in any one of SEQ ID NOs: 130, 145, 146, or 147.

In other embodiments, the ADC gene may be derived from a C. acetobutylicum panD gene encoding the amino acid sequence set forth in SEQ ID NO: 18. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 18. In certain embodiments, a C. acetobutylicum-derived ADC gene may comprise the nucleotide sequence set forth in SEQ ID NO: 131, or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 131.

In other embodiments, the ADC gene may be derived from a H. pylori ADC gene encoding the amino acid sequence set forth in SEQ ID NO: 133. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 133. In certain embodiments, a H. pylori-derived ADC gene may comprise the nucleotide sequence set forth in SEQ ID NO: 133, or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 133.

In other embodiments, the ADC gene may be derived from a Bacillus sp. TS25 ADC gene encoding the amino acid sequence set forth in SEQ ID NO: 135. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 135. In certain embodiments, a Bacillus sp. TS25-derived ADC gene may comprise the nucleotide sequence set forth in SEQ ID NO: 134, or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 134.

In other embodiments, the ADC gene may be derived from a C. glutamicum ADC gene encoding the amino acid sequence set forth in SEQ ID NO: 137. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 137. In certain embodiments, a C. glutamicum-derived ADC gene may comprise the nucleotide sequence set forth in SEQ ID NO: 136, or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 136.

In other embodiments, the ADC gene may be derived from a B. licheniformis ADC gene encoding the amino acid sequence set forth in SEQ ID NO: 139. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 139. In certain embodiments, a B. licheniformis-derived ADC gene may comprise the nucleotide sequence set forth in any one of SEQ ID NOs: 138, 148, 149, 150, or 151; or a nucleotide sequence with at least 50%, at least 60%, at least 70%, at least 80%, at least 85%, at least 90%, at least 95%, at least 97%, or at least 99% sequence identity to the nucleotide sequence set forth in any one of SEQ ID NOs: 138, 148, 149, 150, or 151.

In other embodiments, the ADC gene may be derived from the Class Insecta (insect) ADC gene, e.g., as described in WO 2015/017721 (the content of which is incorporated herein by reference). For example, in some embodiments, the ADC gene may encode an amino acid sequence of the Aedes aegypti ADC of SEQ ID NO: 162, the Culex quinquefasciatus ADC of SEQ ID NO: 163, the Anopheles gambiae ADC of SEQ ID NO: 164, the Tribolium castaneum ADC of SEQ ID NO: 165, the Attagenus smirnovi ADC of SEQ ID NO: 166, the Acyrthosiphon pisum ADC of SEQ ID NO: 167, the Drosophila sechellia ADC of SEQ ID NO: 168, the Drosophila melanogaster ADC of SEQ ID NO: 169, the Danaus plexippus ADC of SEQ ID NO: 170, the Drosophila yakuba ADC of SEQ ID NO: 171, the Drosophila erecta ADC of SEQ ID NO: 172, the Papilio xuthus ADC of SEQ ID NO: 173, the Drosophila persimilis ADC of SEQ ID NO: 174, the Bombyx mori ADC of SEQ ID NO: 175, the Drosophila ananassae ADC of SEQ ID NO: 176, the Drosophila mojavensis ADC of SEQ ID NO: 177, the Drosophila grimshawi ADC of SEQ ID NO: 178, the Biston betularia ADC of SEQ ID NO: 179, the Drosophila willistoni ADC of SEQ ID NO: 180, the Apis mellifera ADC of SEQ ID NO: 181, the Drosophila virilis ADC of SEQ ID NO: 182, the Nasonia vitripennis ADC of SEQ ID NO: 183, the Bicyclus anynana ADC of SEQ ID NO: 184, the Harpegnathos saltator ADC of SEQ ID NO: 185, the Acromyrmex echinatior ADC of SEQ ID NO: 186, the Camponotus floridanus ADC of SEQ ID NO: 187, the Pediculus humanus ADC of SEQ ID NO: 188, the Atta cephalotes ADC of SEQ ID NO: 189, the Meligethes aeneus ADC of SEQ ID NO: 190, or the Solenopsis invicta ADC of SEQ ID NO: 191. In another aspect is a recombinant cell comprising an aspartate 1-decarboxylase (ADC) of the Class Bivalvia, Branchiopoda, Gastropoda, or Leptocardii, such as the Daphnia pulex ADC of SEQ ID NO: 192, the Lottia gigantea ADC of SEQ ID NO: 193, the Branchiostoma floridae ADC of SEQ ID NO: 194, or the Crassostrea gigas ADC of SEQ ID NO: 195. In some embodiments, the ADC gene may encode an amino acid sequence with at least 50%, at least 60%, at least 70%, at least 80%, at least 85%, at least 90%, at least 95%, at least 97%, or at least 99% sequence identity to the amino acid sequence of any one of SEQ ID NOs: 162-195.

In addition to the aminotransferase (e.g., BAAT) comprising the motif (A,C)-Xn-(GSLS)-Xn-SKA(L/l)(L/l) described herein (e.g., SEQ ID NO: 206, 207, 208, 209, 218, 220, 222, 224, 226, 228, 230, 232, or 234), the recombinant cell may optionally comprise one or more (e.g., two, several) additional aminotransferase gene(s), such as an additional BAAT gene. For example, the additional BAAT gene may be derived from the I. orientalis homolog to the pyd4 gene encoding the amino acid sequence set forth in SEQ ID NO: 20. In some embodiments, the additional BAAT gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 20. In certain embodiments, an additional I. orientalis-derived BAAT gene may comprise the nucleotide sequence set forth in SEQ ID NO: 19 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 19. In other embodiments, the additional BAAT gene may be derived from the S. kluyveri pyd4 gene encoding the amino acid sequence set forth in SEQ ID NO: 21. In some embodiments, the additional BAAT gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 21. In certain embodiments, an additional S. kluyveri-derived BAAT gene may comprise the nucleotide sequence set forth in SEQ ID NO: 142 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 142. In other embodiments, the additional BAAT gene may be derived from a bacterial source. For example, an additional BAAT gene may be derived from an S. avermitilis BAAT gene encoding the amino acid sequence set forth in SEQ ID NO: 22. In some embodiments, the additional BAAT gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 22. In certain embodiments, an additional S. avermitilis-derived BAAT gene may comprise the nucleotide sequence set forth in SEQ ID NO: 140 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 140.

In some embodiments, the additional BAAT gene may be derived from the S. avermitilis gabT encoding the amino acid sequence set forth in SEQ ID NO: 23. In some embodiments, the S. avermitilis-derived BAAT gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 23. In other embodiments, an additional BAAT gene may be derived from the S. cerevisiae gabT gene uga1 encoding the amino acid sequence set forth in SEQ ID NO: 24. In some embodiments, the S. cerevisiae-derived BAAT gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 24. In certain embodiments, an S. cerevisiae-derived BAAT gene may comprise the nucleotide sequence set forth in SEQ ID NO: 141 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 141.

A “3-HP dehydrogenase gene” or “3-HPDH gene” as used herein refers to any gene that encodes a polypeptide with 3-HP dehydrogenase activity, meaning the ability to catalyze the conversion of malonate semialdehyde to 3-HP. Enzymes having 3-HP dehydrogenase activity are classified as EC 1.1.1.59 if they utilize a NAD(H) cofactor, and as EC 1.1.1.298 if they utilize a NADP(H) cofactor. Enzymes classified as EC 1.1.1.298 are alternatively referred to as malonate semialdehyde reductases.

In certain embodiments, the 3-HPDH gene may be derived from a bacterial source. For example, a 3-HPDH gene may be derived from an E. coli ydfG gene encoding the amino acid sequence in SEQ ID NO: 27. In some embodiments, the 3-HPDH gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 27. In certain embodiments, the E. coli-derived 3-HPDH gene may comprise the nucleotide sequence set forth in SEQ ID NO: 143 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 143. In other embodiments, the 3-HPDH gene may be derived from an M. sedula malonate semialdehyde reductase gene encoding the amino acid sequence set forth in SEQ ID NO: 29. In some embodiments, the 3-HPDH gene may encode an amino acid sequence with at least 50%, at least 60%, at least 70%, at least 80%, at least 85%, at least 90%, at least 95%, at least 97%, or at least 99% sequence identity to the amino acid sequence set forth in SEQ ID NO: 29. In certain embodiments, the M. sedula-derived 3-HPDH gene may comprise the nucleotide sequence set forth in SEQ ID NO: 152 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 152.

In certain embodiments, the 3-HPDH gene may be derived from a yeast source. For example, the 3-HPDH may be derived from the S. cerevisiae YMR226C gene encoding the amino acid sequence set forth in SEQ ID NO: 129. In some embodiments, the 3-HPDH gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 129. In certain embodiments, the S. cerevisiae-derived 3-HPDH gene may comprise the nucleotide sequence set forth in SEQ ID NO: 144 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 144. In other embodiments, the 3-HPDH gene may encode an amino acid sequence with at least 50%, at least 60%, at least 70%, at least 80%, at least 85%, at least 90%, at least 95%, at least 97%, or at least 99% sequence identity to the Candida parapsilosis amino acid sequence of SEQ ID NO: 197 (or amino acids 17-354 of SEQ ID NO: 197). In other embodiments, the 3-HPDH gene may be derived from the I. orientalis homolog to the YMR226C gene encoding the amino acid sequence set forth in SEQ ID NO: 26. In some embodiments, the 3-HPDH gene may encode an amino acid sequence with at least 50%, at least 60%, at least 70%, at least 80%, at least 85%, at least 90%, at least 95%, at least 97%, or at least 99% sequence identity to the amino acid sequence of SEQ ID NO: 26. In certain embodiments, the I. orientalis-derived 3-HPDH gene may comprise the nucleotide sequence set forth in SEQ ID NO: 25 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 25. In other embodiments, the 3-HPDH gene may encode an amino acid sequence with at least 50%, at least 60%, at least 70%, at least 80%, at least 85%, at least 90%, at least 95%, at least 97%, or at least 99% sequence identity to the Debaryomyces hansenii amino acid sequence of SEQ ID NO: 199. In other embodiments, the 3-HPDH gene may encode an amino acid sequence with at least 50%, at least 60%, at least 70%, at least 80%, at least 85%, at least 90%, at least 95%, at least 97%, or at least 99% sequence identity to the Meyerozyma guilliermondii amino acid sequence of SEQ ID NO: 201. In other embodiments, the 3-HPDH gene may encode an amino acid sequence with at least 50%, at least 60%, at least 70%, at least 80%, at least 85%, at least 90%, at least 95%, at least 97%, or at least 99% sequence identity to the Clavispora lusitaniae amino acid sequence of SEQ ID NO: 203.

In certain embodiments, the 3-HPDH gene may be a natural or engineered gene having an increased specificity for NAD(H) compared to NADP(H). 3-HPDH variants having increased specificity for NAD(H) are described in WO 2013/049073 (the content of which is incorporated herein by reference).

A “3-hydroxyisobutyrate dehydrogenase gene” or “HIBADH gene” as used herein refers to any gene that encodes a polypeptide with 3-hydroxyisobutyrate dehydrogenase activity, meaning the ability to catalyze the conversion of 3-hydroxyisobutyrate to methylmalonate semialdehyde. Enzymes having 3-hydroxyisobutyrate dehydrogenase activity are classified as EC 1.1.1.31. Some 3-hydroxyisobutyrate dehydrogenases also have 3-HPDH activity. In certain embodiments, an HIBADH gene may be derived from a bacterial source. For example, an HIBADH gene may be derived from an A. faecalis M3A gene encoding the amino acid sequence set forth in SEQ ID NO: 28, a P. putida KT2440 or E23440 mmsB gene encoding the amino acid sequence set forth in SEQ ID NO: 30 or SEQ ID NO: 31, respectively, or a P. aeruginosa PAO1 mmsB gene encoding the amino acid sequence set forth in SEQ ID NO: 32. In certain embodiments, an HIBADH gene may encode an amino acid sequence with at least 50%, at least 60%, at least 70%, at least 80%, at least 85%, at least 90%, at least 95%, at least 97%, or at least 99% sequence identity to the amino acid sequence set forth in SEQ ID NOs: 28, 30, 31, or 32.

A “4-hydroxybutyrate dehydrogenase gene” as used herein refers to any gene that encodes a polypeptide with 4-hydroxybutyrate dehydrogenase activity, meaning the ability to catalyze the conversion of 4-hydroxybutanoate to succinate semialdehyde. Enzymes having 4-hydroxybutyrate dehydrogenase activity are classified as EC 1.1.1.61. Some 4-hydroxybutyrate dehydrogenases also have 3-HPDH activity. In certain embodiments, a 4-hydroxybutyrate dehydrogenase gene may be derived from a bacterial source. For example, a 4-hydroxybutyrate dehydrogenase gene may be derived from a R. eutropha H16 4hbd gene encoding the amino acid sequence set forth in SEQ ID NO: 33 or a C. kluyveri DSM 555 hbd gene encoding the amino acid sequence set forth in SEQ ID NO: 34. In other embodiments, the gene may encode an amino acid sequence with at least 50%, at least 60%, at least 70%, at least 80%, at least 85%, at least 90%, at least 95%, at least 97%, or at least 99% sequence identity to the amino acid sequence set forth in SEQ ID NOs: 33 or 34.

A “PEP carboxykinase gene” or “PCK gene” as used herein refers to any gene that encodes a polypeptide with PEP carboxykinase activity, meaning the ability to catalyze the conversion of PEP, CO₂, and ADP or GDP to OAA and ATP or GTP, or vice versa. Enzymes having PEP carboxykinase activity are classified as EC 4.1.1.32 (GTP/GDP utilizing) and EC 4.1.1.49 (ATP/ADP utilizing). In certain embodiments, a PCK gene may be derived from a yeast source. In other embodiments, a PCK gene may be derived from a bacterial source, and in certain of these embodiments the gene may be derived from a bacteria in which the PCK reaction favors the production of OAA rather than the more common form of the reaction where decarboxylation is dominant. For example, a PCK gene may be derived from an M. succiniciproducens PCK gene encoding the amino acid sequence set forth in SEQ ID NO: 35, an A. succiniciproducens PCK gene encoding the amino acid sequence set forth in SEQ ID NO: 36, an A. succinogenes PCK gene encoding the amino acid sequence set forth in SEQ ID NO: 37, or an R. eutropha PCK gene encoding the amino acid sequence set forth in SEQ ID NO: 38. In other embodiments, a PCK gene has undergone one or more mutations versus the native gene from which it was derived, such that the resultant gene encodes a polypeptide that preferably catalyzes the conversion of PEP to OAA. For example, a PCK gene may be derived from an E. coli K12 strain PCK gene encoding the amino acid sequence set forth in SEQ ID NO: 39, where the gene has been mutated to preferably catalyze the conversion of PEP to OAA. In other embodiments the conversion of PEP to OAA is catalyzed by a PEP carboxytransphosphorylase such as is found in propionic acid bacteria (e.g., P. shermanii, A. woodii) which use inorganic phosphate and diphosphate rather than ATP/ADP or GTP/GDP.

A “malate dehydrogenase gene” as used herein refers to any gene that encodes a polypeptide with malate dehydrogenase activity, meaning the ability to catalyze the conversion of OAA to malate. In certain embodiments, a malate dehydrogenase gene may be derived from a bacterial or yeast source.

A “malate decarboxylase gene” as used herein refers to any gene that encodes a polypeptide with malate decarboxylase activity, meaning the ability to catalyze the conversion of malate to 3-HP. Malate decarboxylase activity is not known to occur naturally. Therefore, a malate decarboxylase gene may be derived by incorporating one or more mutations into a native source gene that encodes a polypeptide with acetolactate decarboxylase activity. Polypeptides with acetolactate decarboxylase activity catalyze the conversion of 2-hydroxy-2-methyl-3-oxobutanoate to 2-acetoin, and are classified as EC 4.1.1.5. In certain embodiments, a malate decarboxylase gene may be derived from a bacterial source. For example, a malate decarboxylase gene may be derived from an L. lactis aldB gene encoding the amino acid sequence set forth in SEQ ID NO: 40, an S. thermophilus aldB gene encoding the amino acid sequence set forth in SEQ ID NO: 41, a B. brevis aldB gene encoding the amino acid sequence set forth in SEQ ID NO: 42, or a E. aerogenes budA gene encoding the amino acid sequence set forth in SEQ ID NO: 43.

An “alpha-ketoglutarate (AKG) decarboxylase gene” or “KGD gene” as used herein refers to any gene that encodes a polypeptide with alpha-ketoglutarate decarboxylase activity, meaning the ability to catalyze the conversion of alpha-ketoglutarate (2-oxoglutarate) to succinate semialdehyde. Enzymes having AKG decarboxylase activity are classified as EC 4.1.1.71. A KGD gene may be used to derive a gene encoding a polypeptide capable of catalyzing the conversion of OAA to malonate semialdehyde. This activity may be found in a native KGD gene, or it may derived by incorporating one or more mutations into a native KGD gene. In certain embodiments, a KGD gene may be derived from a bacterial source. For example, a KGD gene may be derived from a M. tuberculosis KGD gene encoding the amino acid sequence set forth in SEQ ID NO: 44, a B. japonicum KGD gene encoding the amino acid sequence set forth in SEQ ID NO: 45, or a M. loti (aka Rhizobium loti) KGD gene encoding the amino acid sequence set forth in SEQ ID NO: 46.

A “branched-chain alpha-keto acid decarboxylase gene” or “BCKA gene” as used herein refers to any gene that encodes a polypeptide with branched-chain alpha-keto acid decarboxylase activity, which can serve to decarboxylate a range of alpha-keto acids from three to six carbons in length. Enzymes having BCKA activity are classified as EC 4.1.1.72. A BCKA gene may be used to derive a gene encoding a polypeptide capable of catalyzing the conversion of OAA to malonate semialdehyde. This activity may be found in a native BCKA gene, or it may be derived by incorporating one or more mutations into a native BCKA gene. In certain embodiments, a BCKA gene may be derived from a bacterial source. For example, a BCKA gene may be derived from a L. lactis kdcA gene encoding the amino acid sequence set forth in SEQ ID NO: 47.

An “indolepyruvate decarboxylase gene” or “IPDA gene” as used herein refers to any gene that encodes a polypeptide with indolepyruvate decarboxylase activity, meaning the ability to catalyze the conversion of indolepyruvate to indoleacetaldehyde. Enzymes having IPDA activity are classified as EC 4.1.1.74. An IPDA gene may be used to derive a gene encoding a polypeptide capable of catalyzing the conversion of OAA to malonate semialdehyde. This activity may be found in a native IPDA gene, or it may be derived by incorporating one or more mutations into a native IPDA gene. In certain embodiments, an indolepyruvate decarboxylase gene may be derived from a yeast, bacterial, or plant source.

A “pyruvate decarboxylase gene” or “PDC gene” as used herein refers to any gene that encodes a polypeptide with pyruvate decarboxylase activity, meaning the ability to catalyze the conversion of pyruvate to acetaldehyde. Enzymes having PDC activity are classified as EC 4.1.1.1. In preferred embodiments, a PDC gene that is incorporated into a recombinant cell as provided herein has undergone one or more mutations versus the native gene from which it was derived such that the resultant gene encodes a polypeptide capable of catalyzing the conversion of OAA to malonate semialdehyde. In certain embodiments, a PDC gene may be derived from a bacterial source. For example, a PDC gene may be derived from a Z. mobilis PDC gene encoding the amino acid sequence set forth in SEQ ID NO: 52 or an A. pasteurianus PDC gene encoding the amino acid sequence set forth in SEQ ID NO: 53. In other embodiments, a PDC gene may be derived from a yeast source. For example, a PDC gene may be derived from an S. cerevisiae PDC1 gene encoding the amino acid sequence set forth in SEQ ID NO: 50, an I. orientalis PDC gene encoding the amino acid sequence set forth in SEQ ID NO: 49, or a K. lactis PDC encoding the amino acid sequence set forth in SEQ ID NO: 51. In certain embodiments, a PDC gene derived from the I. orientalis PDC gene may comprise the nucleotide sequence set forth in SEQ ID NO: 48 or a nucleotide sequence with at least 50%, at least 60%, at least 70%, at least 80%, at least 85%, at least 90%, at least 95%, at least 97%, or at least 99% sequence identity to the nucleotide sequence set forth in SEQ ID NO: 48.

A “benzoylformate decarboxylase” gene as used herein refers to any gene that encodes a polypeptide with benzoylformate decarboxylase activity, meaning the ability to catalyze the conversion of benzoylformate to benzaldehyde. Enzymes having benzoylformate decarboxylase activity are classified as EC 4.1.1.7. In preferred embodiments, a benzoylformate decarboxylase gene that is incorporated into a recombinant cell as provided herein has undergone one or more mutations versus the native gene from which it was derived such that the resultant gene encodes a polypeptide capable of catalyzing the conversion of OAA to malonate semialdehyde. In certain embodiments, a benzoylformate decarboxylase gene may be derived from a bacterial source. For example, a benzoylformate decarboxylase gene may be derived from a P. putida mdlC gene encoding the amino acid sequence set forth in SEQ ID NO: 54, a P. aeruginosa mdlC gene encoding the amino acid sequence set forth in SEQ ID NO: 55, a P. stutzeri dpgB gene encoding the amino acid sequence set forth in SEQ ID NO: 56, or a P. fluorescens ilvB-1 gene encoding the amino acid sequence set forth in SEQ ID NO: 57.

An “OAA formatelyase gene” as used herein refers to any gene that encodes a polypeptide with OAA formatelyase activity, meaning the ability to catalyze the conversion of an acylate ketoacid to its corresponding CoA derivative. A polypeptide encoded by an OAA formatelyase gene may have activity on pyruvate or on another ketoacid. In certain embodiments, an OAA formatelyase gene encodes a polypeptide that converts OAA to malonyl-CoA.

A “malonyl-CoA reductase gene” as used herein refers to any gene that encodes a polypeptide with malonyl-CoA reductase activity, meaning the ability to catalyze the conversion of malonyl-CoA to malonate semialdehyde (also referred to as Co-A acylating malonate semialdehyde dehydrogenase activity). In certain embodiments, a malonyl-CoA reductase gene may be derived from a bifunctional malonyl-CoA reductase gene which also has the ability to catalyze the conversion of malonate semialdehyde to 3-HP. In certain of these embodiments, a malonyl-CoA reductase gene may be derived from a bacterial source. For example, a malonyl-CoA reductase gene may be derived from a C. aurantiacus malonyl-CoA reductase gene encoding the amino acid sequence set forth in SEQ ID NO: 58, an R. castenholzii malonyl-CoA reductase gene encoding the amino acid sequence set forth in SEQ ID NO: 59, or an Erythrobacter sp. NAP1 malonyl-CoA reductase gene encoding the amino acid sequence set forth in SEQ ID NO: 60. In other embodiments, a malonyl-CoA reductase gene may be derived from a malonyl-CoA reductase gene encoding a polypeptide that only catalyzes the conversion of malonyl-CoA to malonate semialdehyde. For example, a malonyl-CoA reductase gene may be derived from an M. sedula Msed_0709 gene encoding the amino acid sequence set forth in SEQ ID NO: 61 or a S. tokodaii malonyl-CoA reductase encoding the amino acid sequence set forth in SEQ ID NO: 62.

A “pyruvate dehydrogenase gene” or “PDH gene” as used herein refers to any gene that encodes a polypeptide with pyruvate dehydrogenase activity, meaning the ability to catalyze the conversion of pyruvate to acetyl-CoA. In certain embodiments, a PDH gene may be derived from a yeast source. For example, a PDH gene may be derived from an S. cerevisiae LAT1, PDA1, PDB1, or LPD gene encoding the amino acid sequence set forth in SEQ ID NOs: 63-66, respectively. In other embodiments, a PDH gene may be derived from a bacterial source. For example, a PDH gene may be derived from an E. coli strain K12 substr. MG1655 aceE, aceF, or lpd gene encoding the amino acid sequence set forth in SEQ ID NOs: 67-69, respectively, or a B. subtilis pdhA, pdhB, pdhC, or pdhD gene encoding the amino acid sequence set forth in SEQ ID NOs: 70-73, respectively.

An “acetyl-CoA carboxylase gene” or “ACC gene” as used herein refers to any gene that encodes a polypeptide with acetyl-CoA carboxylase activity, meaning the ability to catalyze the conversion of acetyl-CoA to malonyl-CoA. Enzymes having acetyl-CoA carboxylase activity are classified as EC 6.4.1.2. In certain embodiments, an acetyl-CoA carboxylase gene may be derived from a yeast source. For example, an acetyl-CoA carboxylase gene may be derived from an S. cerevisiae ACC1 gene encoding the amino acid sequence set forth in SEQ ID NO: 74. In other embodiments, an acetyl-CoA carboxylase gene may be derived from a bacterial source. For example, an acetyl-CoA carboxylase gene may be derived from an E. coli accA, accB, accC, or accD gene encoding the amino acid sequence set forth in SEQ ID NOs: 75-78, respectively, or a C. aurantiacus accA, accB, accC, or accD gene encoding the amino acid sequence set forth in SEQ ID NOs: 79-82, respectively.

An “alanine dehydrogenase gene” as used herein refers to any gene that encodes a polypeptide with alanine dehydrogenase activity, meaning the ability to catalyze the NAD-dependent reductive amination of pyruvate to alanine. Enzymes having alanine dehydrogenase activity are classified as EC 1.4.1.1. In certain embodiments, an alanine dehydrogenase gene may be derived from a bacterial source. For example, an alanine dehydrogenase gene may be derived from a B. subtilis alanine dehydrogenase gene encoding the amino acid sequence set forth in SEQ ID NO: 83.

A “pyruvate/alanine aminotransferase gene” as used herein refers to any gene that encodes a polypeptide with pyruvate/alanine aminotransferase activity, meaning the ability to catalyze the conversion of pyruvate and L-glutamate to alanine and 2-oxoglutarate. In certain embodiments, a pyruvate/alanine aminotransferase gene is derived from a yeast source. For example, a pyruvate/alanine aminotransferase gene may be derived from an S. pombe pyruvate/alanine aminotransferase gene encoding the amino acid sequence set forth in SEQ ID NO: 84 or an S. cerevisiae ALT2 gene encoding the amino acid sequence set forth in SEQ ID NO: 85.

An “alanine 2,3 aminomutase gene” or “AAM gene” as used herein refers to a gene that encodes a polypeptide with alanine 2,3 aminomutase activity, meaning the ability to catalyze the conversion of alanine to β-alanine. Alanine 2,3 aminomutase activity is not known to occur naturally. Therefore, an alanine 2,3 aminomutase gene can be derived by incorporating one or more mutations into a native source gene that encodes a polypeptide with similar activity such as lysine 2,3 aminomutase activity (see, e.g., U.S. Pat. No. 7,309,597). In certain embodiments, the native source gene may be a B. subtilis lysine 2,3 aminomutase gene encoding the amino acid sequence set forth in SEQ ID NO: 86, a P. gingivalis lysine 2,3 aminomutase gene encoding the amino acid sequence set forth in SEQ ID NO: 87, or a F. nucleatum (ATCC-10953) lysine 2,3 aminomutase gene encoding the amino acid sequence set forth in SEQ ID NO: 88.

A “CoA transferase gene” as used herein refers to any gene that encodes a polypeptide with CoA transferase activity, which in one example includes the ability to catalyze the conversion of β-alanine to β-alanyl-CoA and/or the conversion of lactate to lactyl-CoA. In certain embodiments, a CoA transferase gene may be derived from a yeast source. In other embodiments, a CoA transferase gene may be derived from a bacterial source. For example, a CoA transferase gene may be derived from an M. elsdenii CoA transferase gene encoding the amino acid sequence set forth in SEQ ID NO: 89.

A “CoA synthetase gene” as used herein refers to any gene that encodes a polypeptide with CoA synthetase activity. In one example this includes the ability to catalyze the conversion of β-alanine to β-alanyl-CoA. In another example, this includes the ability to catalyze the conversion of lactate to lactyl-CoA. In certain embodiments, a CoA synthetase gene may be derived from a yeast source. For example, a CoA synthetase gene may be derived from an S. cerevisiae CoA synthetase gene. In other embodiments, a CoA synthetase gene may be derived from a bacterial source. For example, a CoA synthetase gene may be derived from an E. coli CoA synthetase, R. sphaeroides, or S. enterica CoA synthetase gene.

A “β-alanyl-CoA ammonia lyase gene” as used herein refers to any gene that encodes a polypeptide with β-alanyl-CoA ammonia lyase activity, meaning the ability to catalyze the conversion of β-alanyl-CoA to acrylyl-CoA. In certain embodiments, a β-alanyl-CoA ammonia lyase gene may be derived from a bacterial source, such as a C. propionicum β-alanyl-CoA ammonia lyase gene encoding the amino acid sequence set forth in SEQ ID NO: 90.

A “3-HP-CoA dehydratase gene” or “acrylyl-CoA hydratase gene” as used herein refers to any gene that encodes a polypeptide with 3-HP-CoA dehydratase gene activity, meaning the ability to catalyze the conversion of acrylyl-CoA to 3-HP-CoA. Enzymes having 3-HP-CoA dehydratase activity are classified as EC 4.2.1.116. In certain embodiments, a 3-HP-CoA dehydratase gene may be derived from a yeast or fungal source, such as a P. sojae 3-HP-CoA dehydratase gene encoding the amino acid sequence set forth in SEQ ID NO: 91. In other embodiments, a 3-HP-CoA dehydratase gene may be derived from a bacterial source. For example, a 3-HP-CoA dehydratase gene may be derived from a C. aurantiacus 3-HP-CoA dehydratase gene encoding the amino acid sequence set forth in SEQ ID NO: 92, an R. rubrum 3-HP-CoA dehydratase gene encoding the amino acid sequence set forth in SEQ ID NO: 93, or an R. capsulates 3-HP-CoA dehydratase gene encoding the amino acid sequence set forth in SEQ ID NO: 94. In still other embodiments, a 3-HP-CoA dehydratase gene may be derived from a mammalian source. For example, a 3-HP-CoA dehydratase gene may be derived from a H. sapiens 3-HP-CoA dehydratase gene encoding the amino acid sequence set forth in SEQ ID NO: 95.

A “3-HP-CoA hydrolase gene” as used herein refers to any gene that encodes a polypeptide with 3-HP-CoA hydrolase activity, meaning the ability to catalyze the conversion of 3-HP-CoA to 3-HP. In certain embodiments, a 3-HP-CoA gene may be derived from a yeast or fungal source. In other embodiments, a 3-HP-CoA gene may be derived from a bacterial or mammalian source.

A “3-hydroxyisobutyryl-CoA hydrolase gene” as used herein refers to any gene that encodes a polypeptide with 3-hydroxyisobutyryl-CoA hydrolase activity, which in one example includes the ability to catalyze the conversion of 3-HP-CoA to 3-HP. In certain embodiments, a 3-hydroxyisobutyryl-CoA hydrolase gene may be derived from a bacterial source, such as a P. fluorescens 3-hydroxyisobutyryl-CoA hydrolase gene encoding the amino acid sequence set forth in SEQ ID NO: 96 or a B. cereus 3-hydroxyisobutyryl-CoA hydrolase gene encoding the amino acid sequence set forth in SEQ ID NO: 97. In other embodiments, a 3-hydroxyisobutyryl-CoA hydrolase gene may be derived from a mammalian source, such as a H. sapiens 3-hydroxyisobutyryl-CoA hydrolase gene encoding the amino acid sequence set forth in SEQ ID NO: 98.

A “lactate dehydrogenase gene” or “LDH gene” as used herein refers to any gene that encodes a polypeptide with lactate dehydrogenase activity, meaning the ability to catalyze the conversion of pyruvate to lactate. In certain embodiments, an LDH gene may be derived from a fungal, bacterial, or mammalian source.

A “lactyl-CoA dehydratase gene” as used herein refers to any gene that encodes a polypeptide with lactyl-CoA dehydratase activity, meaning the ability to catalyze the conversion of lactyl-CoA to acrylyl-CoA. In certain embodiments, a lactyl-CoA dehydratase gene may be derived from a bacterial source. For example, a lactyl-CoA dehydratase gene may be derived from an M. elsdenii lactyl-CoA dehydratase E1, Ella, or Ellb subunit gene encoding the amino acid sequence set forth in SEQ ID NOs: 99-101.

An “aldehyde dehydrogenase gene” as used herein refers to any gene that encodes a polypeptide with aldehyde dehydrogenase activity, which in one example includes the ability to catalyze the conversion of 3-HPA to 3-HP and vice versa. In certain embodiments, an aldehyde dehydrogenase may be derived from a bacterial source, such as an E. coli aldH gene encoding the amino acid sequence set forth in SEQ ID NO: 103 or a K. pneumoniae aldehyde dehydrogenase gene encoding the amino acid sequence set forth in SEQ ID NO: 104. In other embodiments, an aldehyde dehydrogenase gene may be derived from a yeast source, such as an S. cerevisiae aldehyde dehydrogenase gene encoding the amino acid sequence set forth in SEQ ID NO: 102 or an I. orientalis aldehyde dehydrogenase gene encoding the amino acid sequence set forth in SEQ ID NOs: 122, 124, or 126.

A “glycerol dehydratase gene” as used herein refers to any gene that encodes a polypeptide with glycerol dehydratase activity, meaning the ability to catalyze the conversion of glycerol to 3-HPA. In certain embodiments, a glycerol dehydratase gene may be derived from a bacterial source, such as a K. pneumonia or C. freundii glycerol dehydratase gene.

The enzymes of the selected active 3-HP pathway, and activities thereof, can be detected using methods known in the art or as described herein. These detection methods may include use of specific antibodies, formation of an enzyme product, or disappearance of an enzyme substrate. See, for example, Sambrook et al., Molecular Cloning: A Laboratory Manual, Third Ed., Cold Spring Harbor Laboratory, New York (2001); Ausubel et al., Current Protocols in Molecular Biology, John Wiley and Sons, Baltimore, Md. (1999); and Hanai et al., Appl. Environ. Microbiol. 73:7814-7818 (2007)).

The recombinant cells described herein can further contain lipase or esterase activity, for example due to expression of a heterologous polynucleotide encoding a lipase or esterase (EC 3.1.1.-). Such cells can be used to produce an ester of 3-HP, such as methyl 3-hydroxypropionate, ethyl 3-hydroxypropionate, propyl 3-hydroxypropionate, butyl 3-hydroxypropionate, or 2-ethylhexyl 3-hydroxypropionate. The cells can further contain esterase activity, for example due to expression of a heterologous polynucleotide encoding an esterase. Such cells can be used to produce polymerized 3-HP. The cells can further contain alcohol dehydrogenase activity (EC 1.1.1.1), aldehyde dehydrogenase activity (EC 1.2.1.-), or both, for example due to expression of a heterologous polynucleotide encoding an alcohol dehydrogenase, aldehyde dehydrogenase, or both. Such cells can be used to produce 1,3-propanediol.

### Gene Disruptions

The recombinant cell may also comprise one or more (e.g., two, several) gene disruptions, e.g., to divert sugar metabolism from undesired products to 3-HP. In some aspects, the recombinant host cells produce a greater amount of 3-HP compared to the cell without the one or more disruptions when cultivated under identical conditions. In some aspects, one or more of the disrupted endogenous genes are inactivated.

In certain embodiments, the recombinant cells provided herein comprise a disruption of one or more endogenous genes encoding an enzyme involved in ethanol fermentation, including for example pyruvate decarboxylase (PDC, converts pyruvate to acetaldehyde) and/or alcohol dehydrogenase (ADH, converts acetaldehyde to ethanol) genes. These modifications decrease the ability of the cell to produce ethanol, thereby maximizing 3-HP production. However, in certain embodiments the recombinant cells provided herein may be engineered to co-produce 3-HP and ethanol. In those embodiments, endogenous genes encoding an enzyme involved in ethanol fermentation are preferably not disrupted, and in certain embodiments the cells may comprise one or more heterologous genes that increase ethanol production.

In some embodiments, the recombinant cells comprise a disruption to an endogenous gene encoding a PDC having at least 75%, e.g., at least 80%, at least 85%, at least 90%, at least 95%, at least 96%, at least 97%, at least 98%, at least 99%, or 100% sequence identity to SEQ ID NO: 154. In some embodiments, the endogenous gene encodes a PDC having an amino acid sequence comprising or consisting of SEQ ID NO: 154. In some embodiments, the coding sequence of the endogenous gene encoding the PDC has at least 75%, e.g., at least 80%, at least 85%, at least 90%, at least 95%, at least 96%, at least 97%, at least 98%, at least 99%, or 100% sequence identity to SEQ ID NO: 153. In some embodiments, the coding sequence of the endogenous gene encoding the PDC comprises or consists of SEQ ID NO: 153. In some embodiments, the endogenous gene encoding the PDC is inactivated.

In certain embodiments, the recombinant cells provided herein comprise a disruption of one or more endogenous genes encoding enzymes involved in producing alternate fermentative products such as glycerol or other byproducts such as lactate, acetate or diols. For example, the cells provided herein may comprise a disruption of one or more of glycerol 3-phosphate dehydrogenase (GPD, catalyzes reaction of dihydroxyacetone phosphate to glycerol 3-phosphate), pyruvate reductase (catalyzes the reduction of pyruvate to D-lactate), glycerol 3-phosphatase (GPP, catalyzes conversion of glycerol-3 phosphate to glycerol), glycerol kinase (catalyzes conversion of glycerol 3-phosphate to glycerol), dihydroxyacetone kinase (catalyzes conversion of dihydroxyacetone phosphate to dihydroxyacetone), glycerol dehydrogenase (catalyzes conversion of dihydroxyacetone to glycerol), aldehyde dehydrogenase (ALD, e.g., converts acetaldehyde to acetate or 3-HP to 3-HPA), and butanediol dehydrogenase (catalyzes conversion of butanediol to acetoin and vice versa) genes.

In some embodiments, the recombinant cells comprise a disruption to an endogenous gene encoding a GPD having at least 75%, e.g., at least 80%, at least 85%, at least 90%, at least 95%, at least 96%, at least 97%, at least 98%, at least 99%, or 100% sequence identity to SEQ ID NO: 156. In some embodiments, the endogenous gene encodes a GPD having an amino acid sequence comprising or consisting of SEQ ID NO: 156. In some embodiments, the coding sequence of the endogenous gene encoding the GPD has at least 75%, e.g., at least 80%, at least 85%, at least 90%, at least 95%, at least 96%, at least 97%, at least 98%, at least 99%, or 100% sequence identity to SEQ ID NO: 155. In some embodiments, the coding sequence of the endogenous gene encoding the GPD comprises or consists of SEQ ID NO: 155. In some embodiments, the endogenous gene encoding the GPD is inactivated.

In some embodiments, the recombinant cells comprise a disruption to an endogenous gene encoding a pyruvate reductase having at least 75%, e.g., at least 80%, at least 85%, at least 90%, at least 95%, at least 96%, at least 97%, at least 98%, at least 99%, or 100% sequence identity to SEQ ID NO: 205 (e.g., as described in U.S. Ser. No. 62/126,377, filed Feb. 27, 2015, the content of which is hereby incorporated by reference). In some embodiments, the endogenous gene encodes a pyruvate reductase having an amino acid sequence comprising or consisting of SEQ ID NO: 205. In some embodiments, the coding sequence of the endogenous gene encoding the pyruvate reductase has at least 75%, e.g., at least 80%, at least 85%, at least 90%, at least 95%, at least 96%, at least 97%, at least 98%, at least 99%, or 100% sequence identity to SEQ ID NO: 204. In some embodiments, the coding sequence of the endogenous gene encoding the pyruvate reductase comprises or consists of SEQ ID NO: 204. In some embodiments, the endogenous gene encoding the pyruvate reductase is inactivated.

In certain embodiments, the recombinant cells provided herein comprise a disruption of one or more endogenous genes encoding enzymes that catalyze a reverse reaction in a 3-HP pathway, including for example PEP carboxykinase (PCK), enzymes with OAA decarboxylase activity, or CYB2A or CYB2B (catalyzes the conversion of lactate to pyruvate). PCK catalyzes the conversion of PEP to OAA and vice versa, but exhibits a preference for the OAA to PEP reaction. To reduce the conversion of OAA to PEP, one or more copies of a native PCK gene may be disrupted. In certain embodiments, cells in which one or more native PCK genes have been disrupted may express one or more heterologous PCK genes that have been mutated to encode a polypeptide favoring the conversion of PEP to OAA. OAA decarboxylase catalyzes the conversion of OAA to pyruvate. Enzymes with OAA decarboxylase activity have been identified, such as that coded by the Entner-Doudoroff aldolase (eda) gene in E. coli and malic enzyme (MAE) in yeast and fungi. To reduce OAA decarboxylase activity, one or more copies of a native gene encoding an enzyme with OAA decarboxylase activity may be disrupted. In certain embodiments, cells in which one or more native OAA decarboxylation genes have been disrupted may express one or more heterologous OAA decarboxylation genes that have been mutated to encode a polypeptide that catalyzes the conversion of pyruvate to OAA.

In certain embodiments, the recombinant cells provided herein comprise a disruption to one or more genes selected from dse2, scw11, eaf3, sed1, and sam2. (See, e.g., Suzuki et al, J. Biosci. Bioeng. 2013, 115, 467-474; and Dato et al., Microbial Cell Factories, 2014, 13, 147).

In certain embodiments, the recombinant cells provided herein comprise a disruption of one or more endogenous genes encoding an enzyme involved in an undesirable reaction with a 3-HP pathway product or intermediate. Examples of such genes include those encoding an enzyme that converts 3-HP to an aldehyde of 3-HP, which are known to be toxic to certain cells.

In certain embodiments, the recombinant cells provided herein comprise a disruption of one or more endogenous genes encoding an enzyme that has a neutral effect on a 3-HP pathway, including for example GAL6 (negative regulator of the GAL system that converts galactose to glucose). Disruption of neutral genes allows for insertion of one or more heterologous genes without affecting native pathways.

Modeling can also be used to design gene disruptions that additionally optimize utilization of the pathway (see, for example, U.S. 2002/0012939, U.S. 2003/0224363, U.S. 2004/0029149, U.S. 2004/0072723, U.S. 2003/0059792, U.S. 2002/0168654, U.S. 2004/0009466, and U.S. Pat. No. 7,127,379). Modeling analysis allows reliable predictions of the effects on cell growth of shifting the metabolism towards more efficient production of 3-HP. One exemplary computational method for identifying and designing metabolic alterations favoring biosynthesis of a desired product is the OptKnock computational framework, Burgard et al., 2003, Biotechnol. Bioeng. 84: 647-657.

The recombinant cells comprising a gene disruption may be constructed using methods well known in the art, including those methods described herein. A portion of the operon can be disrupted such as one or more (e.g., two, several) coding regions or a control sequence required for expression of the coding regions. Such a control sequence of the operon may be a promoter sequence or a functional part thereof, i.e., a part that is sufficient for affecting expression of the operon. For example, a promoter sequence may be inactivated resulting in no expression or a weaker promoter may be substituted for the native promoter sequence to reduce expression of the coding sequences. Other control sequences for possible modification include, but are not limited to, a leader, propeptide sequence, signal sequence, transcription terminator, and transcriptional activator.

The recombinant cells comprising a gene disruption may be constructed by gene deletion techniques to eliminate or reduce expression of the gene. Gene deletion techniques enable the partial or complete removal of the gene thereby eliminating their expression. In such methods, deletion of the gene is accomplished by homologous recombination using a plasmid that has been constructed to contiguously contain the 5′ and 3′ regions flanking the gene.

The recombinant cells comprising a gene disruption may also be constructed by introducing, substituting, and/or removing one or more (e.g., two, several) nucleotides in the gene or a control sequence thereof required for the transcription or translation thereof. For example, nucleotides may be inserted or removed for the introduction of a stop codon, the removal of the start codon, or a frame-shift of the open reading frame. Such a modification may be accomplished by site-directed mutagenesis or PCR generated mutagenesis in accordance with methods known in the art. See, for example, Botstein and Shortle, 1985, Science 229: 4719; Lo et al., 1985, Proc. Natl. Acad. Sci. U.S.A. 81: 2285; Higuchi et al., 1988, Nucleic Acids Res 16: 7351; Shimada, 1996, Meth. Mol. Biol. 57: 157; Ho et al., 1989, Gene 77: 61; Horton et al., 1989, Gene 77: 61; and Sarkar and Sommer, 1990, BioTechniques 8: 404.

The recombinant cells comprising a gene disruption may also be constructed by inserting into the gene a disruptive nucleic acid construct comprising a nucleic acid fragment homologous to the gene that will create a duplication of the region of homology and incorporate construct DNA between the duplicated regions. Such a gene disruption can eliminate gene expression if the inserted construct separates the promoter of the gene from the coding region or interrupts the coding sequence such that a non-functional gene product results. A disrupting construct may be simply a selectable marker gene accompanied by 5′ and 3′ regions homologous to the gene. The selectable marker enables identification of transformants containing the disrupted gene.

The recombinant cells comprising a gene disruption may also be constructed by the process of gene conversion (see, for example, Iglesias and Trautner, 1983, Molecular General Genetics 189: 73-76). For example, in the gene conversion method, a nucleotide sequence corresponding to the gene is mutagenized in vitro to produce a defective nucleotide sequence, which is then transformed into the recombinant strain to produce a defective gene. By homologous recombination, the defective nucleotide sequence replaces the endogenous gene. It may be desirable that the defective nucleotide sequence also comprises a marker for selection of transformants containing the defective sequence.

The recombinant cells comprising a gene disruption may be further constructed by random or specific mutagenesis using methods well known in the art, including, but not limited to, chemical mutagenesis (see, for example, Hopwood, The Isolation of Mutants in Methods in Microbiology (J. R. Norris and D. W. Ribbons, eds.) pp. 363-433, Academic Press, New York, 1970). Modification of the gene may be performed by subjecting the parent strain to mutagenesis and screening for mutant strains in which expression of the gene has been reduced or inactivated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the parent strain to be mutagenized in the presence of the mutagenizing agent of choice under suitable conditions, and selecting for mutants exhibiting reduced or no expression of the gene.

A nucleotide sequence homologous or complementary to a gene described herein may be used from other microbial sources to disrupt the corresponding gene in a recombinant strain of choice.

In one aspect, the modification of a gene in the recombinant cell is unmarked with a selectable marker. Removal of the selectable marker gene may be accomplished by culturing the mutants on a counter-selection medium. Where the selectable marker gene contains repeats flanking its 5′ and 3′ ends, the repeats will facilitate the looping out of the selectable marker gene by homologous recombination when the mutant strain is submitted to counter-selection. The selectable marker gene may also be removed by homologous recombination by introducing into the mutant strain a nucleic acid fragment comprising 5′ and 3′ regions of the defective gene, but lacking the selectable marker gene, followed by selecting on the counter-selection medium. By homologous recombination, the defective gene containing the selectable marker gene is replaced with the nucleic acid fragment lacking the selectable marker gene. Other methods known in the art may also be used.

### Methods of Producing 3-HP and Related Compounds

The recombinant cells described herein may be used for the production of 3-HP. In one aspect is a method of producing 3-HP, comprising: (a) cultivating any one of the recombinant cells described herein (e.g., a recombinant host cell comprising an active 3-HP pathway and a heterologous polynucleotide encoding an aminotransferase) in a medium under suitable conditions to produce the 3-HP; and (b) recovering the 3-HP.

The recombinant cells comprising an active 3-HP pathway may be cultivated in a nutrient medium suitable for 3-HP production using methods well known in the art. For example, the cells may be cultivated by shake flask cultivation, and small-scale or large-scale fermentation (including continuous, batch, fed-batch, or solid state fermentations) in laboratory or industrial fermentors performed in a suitable fermentation medium and under conditions allowing 3-HP production.

The recombinant cells may produce 3-HP in a fermentable medium comprising any one or more (e.g., two, several) sugars, such as glucose, fructose, sucrose, cellobiose, xylose, xylulose, arabinose, mannose, galactose, and/or soluble oligosaccharides. The carbon source may be a twelve carbon sugar such as sucrose, a hexose sugar such as glucose or fructose, glycan or other polymer of glucose, glucose oligomers such as maltose, maltotriose and isomaltotriose, panose, and fructose oligomers. If the cell is modified to impart an ability to ferment pentose sugars, the fermentation medium may include a pentose sugar such as xylose, xylan or other oligomer of xylose, and/or arabinose. Such pentose sugars are suitably hydrolysates of a hemicellulose-containing biomass. In some embodiments, the cell is unable to ferment pentose sugars and/or the fermentable medium comprises less than 1% pentose sugars. In some instances, the fermentable medium is derived from a natural source, such as sugar cane, starch, or cellulose, and may be the result of pretreating the source by enzymatic hydrolysis (saccharification). In some aspects, the fermentable medium comprises sugar cane juice. Suitable media are available from commercial suppliers, may be prepared according to published compositions (e.g., in catalogues of the American Type Culture Collection), or may be prepared from commercially available ingredients.

In addition to the appropriate carbon sources from one or more (e.g., two, several) sugar(s), the fermentable medium may contain other nutrients or stimulators known to those skilled in the art, such as macronutrients (e.g., nitrogen sources) and micronutrients (e.g., vitamins, mineral salts, and metallic cofactors). In some aspects, the carbon source can be preferentially supplied with at least one nitrogen source, such as yeast extract, N₂, peptone (e.g., Bacto™ Peptone), or soytone (e.g., Bacto™ Soytone). Non-limiting examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. Examples of mineral salts and metallic cofactors include, but are not limited to Na, P, K, Mg, S, Ca, Fe, Zn, Mn, Co, and Cu.

In some embodiments, the recombinant cells of the invention can be cultured in a chemically defined medium. In one example, the medium contains around 5 g/L ammonium sulfate, around 3 g/L potassium dihydrogen phosphate, around 0.5 g/L magnesium sulfate, trace elements, and vitamins and around 150 g/L glucose. The pH may be allowed to range freely during cultivation, or may be buffered if necessary to prevent the pH from falling below or rising above predetermined levels. In certain embodiments, the fermentation medium is inoculated with sufficient cells that are the subject of the evaluation to produce an OD₆₀₀ of about 1.0. Unless explicitly noted otherwise, OD₆₀₀ as used herein refers to an optical density measured at a wavelength of 600 nm with a 1 cm pathlength using a model DU600 spectrophotometer (Beckman Coulter).

Specific conditions used for the methods of 3-HP production may be determined by one skilled in the art in light of the teachings herein. In some aspects of the methods, the cells are cultivated for about 12 hours to about 216 hours, such as about 24 hours to about 144 hours, or about 36 hours to about 96 hours. The temperature is typically between about 25° C. to about 60° C., e.g., about 30° C. to about 40° C., about 30° C. to about 40° C., or about 40° C. to about 50° C.

Cultivation may be performed under anaerobic, substantially anaerobic (microaerobic), or aerobic conditions, as appropriate. Briefly, anaerobic refers to an environment devoid of oxygen, substantially anaerobic (microaerobic) refers to an environment in which the concentration of oxygen is less than air, and aerobic refers to an environment wherein the oxygen concentration is approximately equal to or greater than that of the air. Substantially anaerobic conditions include, for example, a culture, batch fermentation or continuous fermentation such that the dissolved oxygen concentration in the medium remains less than 10% of saturation. Substantially anaerobic conditions also includes growing or resting cells in liquid medium or on solid agar inside a sealed chamber maintained with an atmosphere of less than 1% oxygen. The percent of oxygen can be maintained by, for example, sparging the culture with a N₂/CO₂ mixture or other suitable non-oxygen gas or gases. In some embodiments, the cultivation is performed under anaerobic conditions or substantially anaerobic conditions.

In one example, the concentration of cells in the fermentation medium is typically in the range of about 1 to 40, e.g., 2 to 20, or 3 to 10 gram dry cells/liter of fermentation medium during the production phase. If desired, oxygen uptake rate (OUR) can be varied throughout fermentation as a process control (see, e.g., WO03/102200). In some embodiments, the recombinant cells provided herein are cultivated under microaerobic conditions characterized by an oxygen uptake rate from 2 to 45 mmol/L/hr, e.g., 2 to 25, 2 to 20, 2 to 15, 2 to 10, 10 to 45, 15 to 40, 20 to 35, or 25 to 35 mmol/L/hr. In certain embodiments, the recombinant cells provided herein may perform especially well when cultivated under microaerobic conditions characterized by an oxygen uptake rate of from 2 to 25 mmol/L/hr. The medium may be buffered during the production phase such that the pH is maintained in a range of about 3.0 to about 7.0, or from about 4.0 to about 6.0. Suitable buffering agents are basic materials that neutralize the acid as it is formed, and include, for example, calcium hydroxide, calcium carbonate, sodium hydroxide, potassium hydroxide, potassium carbonate, sodium carbonate, ammonium carbonate, ammonia, ammonium hydroxide and the like. In general, those buffering agents that have been used in conventional fermentation processes are also suitable here.

In those embodiments where a buffered fermentation is utilized, acidic fermentation products may be neutralized to the corresponding salt as they are formed. In these embodiments, recovery of the acid involves regeneration of the free acid. This may be done by removing the cells and acidulating the fermentation broth with a strong acid such as sulfuric acid. This results in the formation of a salt by-product. For example, where a calcium salt is utilized as the neutralizing agent and sulfuric acid is utilized as the acidulating agent, gypsum is produced as a salt by-product. This by-product is separated from the broth, and the acid is recovered using techniques such as liquid-liquid extraction, distillation, absorption, and others (see, e.g., T. B. Vickroy, Vol. 3, Chapter 38 of Comprehensive Biotechnology, (ed. M. Moo-Young), Pergamon, Oxford, 1985; Datta et al., 1995, FEMS Microbiol. Rev. 16: 221-231; U.S. Pat. Nos. 4,275,234, 4,771,001, 5,132,456, 5,420,304, 5,510,526, 5,641,406, and 5,831,122, and WO 93/00440.

In other embodiments, the pH of the fermentation medium may be permitted to drop during cultivation from a starting pH that is at or above the pKa of 3-HP, typically 4.5 or higher (such as 5.5, 6.0 or 6.5), to at or below the pKa of the acid fermentation product, e.g., less than 4.5 or 4.0, such as in the range of about 1.5 to about 4.5, in the range of from about 2.0 to about 4.0, or in the range from about 2.0 to about 3.5.

In still other embodiments, fermentation may be carried out to produce a product acid by adjusting the pH of the fermentation broth to at or below the pKa of the product acid prior to or at the start of the fermentation process. The pH may thereafter be maintained at or below the pKa of the product acid throughout the cultivation. In certain embodiments, the pH may be maintained at less than 4.5 or 4.0, such as in a range of about 1.5 to about 4.5, in a range of about 2.0 to about 4.0, or in a range of about 2.0 to about 3.5.

The methods described herein can employ any suitable fermentation operation mode. For example, batch mode fermentation may be used with a close system where culture media and recombinant host cells, set at the beginning of fermentation, have no additional input except for the reagents certain reagents, e.g., for pH control, foam control or others required for process sustenance. The process described herein can also be employed in Fed-batch or continuous mode, as mentioned supra.

The methods described herein may be practiced in several bioreactor configurations, such as stirred tank, bubble column, airlift reactor and others known to those skilled in the art. The methods may be performed in free cell culture or in immobilized cell culture as appropriate. Any material support for immobilized cell culture may be used, such as alginates, fibrous bed, or argyle materials such as chrysotile, montmorillonite KSF and montmorillonite K-10.

In one aspect of the methods, the 3-HP is produced at a titer greater than about 5 g/L, e.g., greater than about 10 g/L, 25 g/L, 50 g/L, 75 g/L, 100 g/L, 125 g/L, 150 g/L, 160 g/L, 170 g/L, 180 g/L, 190 g/L, 200 g/L, 210 g/L, 225 g/L, 250 g/L, 275 g/L, 300 g/L, 325 g/L, 350 g/L, 400 g/L, or 500 g/L; or between about 10 g/L and about 500 g/L, e.g., between about 50 g/L and about 350 g/L, about 100 g/L and about 300 g/L, about 150 g/L and about 250 g/L, about 175 g/L and about 225 g/L, or about 190 g/L and about 210 g/L. In one embodiment, the 3-HP is produced at a titer greater than about 0.01 gram per gram of carbohydrate, e.g., greater than about 0.02, 0.05, 0.75, 0.1, 0.2, 0.3, 0.4, 0.5, 0.6, 0.7, 0.8, 0.9, or 1.0 gram per gram of carbohydrate.

In certain embodiments of the methods provided herein, the recombinant cells produce relatively low levels of ethanol. In certain embodiments, ethanol may be produced in a yield of 10% or less, preferably in a yield of 2% or less. In certain of these embodiments, ethanol is not detectably produced. In other embodiments, however, 3-HP and ethanol may be co-produced. In these embodiments, ethanol may be produced at a yield of greater than 10%, greater than 25%, or greater than 50%.

The 3-HP can be optionally recovered from the fermentation medium using any procedure known in the art including, but not limited to, chromatography (e.g., size exclusion chromatography, adsorption chromatography, ion exchange chromatography), electrophoretic procedures, differential solubility, osmosis, distillation, extraction (e.g., liquid-liquid extraction), pervaporation, extractive filtration, membrane filtration, membrane separation, reverse, or ultrafiltration. In one aspect, the 3-HP is separated from other fermented material and purified by conventional methods of distillation. Accordingly, in one aspect, the method further comprises purifying the recovered 3-HP by distillation.

The recombinant 3-HP may also be purified by the chemical conversion of impurities (contaminants) to products more easily removed from 3-HP by the procedures described above (e.g., chromatography, electrophoretic procedures, differential solubility, distillation, or extraction) and/or by direct chemical conversion of impurities to 3-HP. For example, in one aspect, the method further comprises purifying recovered 3-HP by converting β-alanine contaminant to 3-HP, using chemical techniques known in the art.

In some aspects of the methods, the recombinant 3-HP preparation before and/or after being optionally purified is substantially pure. With respect to the methods of producing 3-HP, “substantially pure” intends a recovered preparation that contains no more than 15% impurity, wherein impurity intends compounds other than 3-HP. In one variation, a substantially pure preparation is provided wherein the preparation contains no more than 25% impurity, or no more than 20% impurity, or no more than 10% impurity, or no more than 5% impurity, or no more than 3% impurity, or no more than 1% impurity, or no more than 0.5% impurity.

3-HP produced using the methods disclosed herein can be converted into other organic compounds (e.g., as described in U.S. Pat. No. 8,030,045 and WO 03/082795, the content of which is incorporated herein by reference). For example, 3-HP can be hydrogenated to form 1,3 propanediol, a valuable polyester monomer. Propanediol also can be created from 3-HP using polypeptides having oxidoreductase activity in vitro or in vivo (e.g., using one or more additional enzymatic activities as described supra). Hydrogenating an organic acid such as 3-HP can be performed using any method such as those used to hydrogenate succinic acid and/or lactic acid. For example, 3-HP can be hydrogenated using a metal catalyst. Accordingly, in one aspect is a method of producing 1,3-propanediol comprising: (a) cultivating a recombinant cell described herein (e.g., a recombinant host cell described herein comprising an active 3-HP pathway and a heterologous polynucleotide encoding an aminotransferase), wherein the recombinant cell further comprises oxidoreductase activity in a medium under suitable conditions to produce 1,3-propanediol; and (b) recovering the 1,3-propanediol. In another aspect is a method of producing 1,3-propanediol, comprising: (a) cultivating a recombinant cell described herein (e.g., a recombinant host cell described herein comprising an active 3-HP pathway and a heterologous polynucleotide encoding an aminotransferase) in a medium under suitable conditions to produce 3-HP; (b) recovering the 3-HP; (c) hydrogenating the 3-HP under suitable conditions to produce 1,3-propanediol; and (d) recovering the 1,3-propanediol.

3-HP may also be converted to an ester of 3-HP, such as methyl 3-hydroxypropionate, ethyl 3-hydroxypropionate, propyl 3-hydroxypropionate, butyl 3-hydroxypropionate, or 2-ethylhexyl 3-hydroxypropionate. In one aspect is a method of producing a 3-HP ester comprising: (a) cultivating a recombinant cell described herein (e.g., a recombinant host cell described herein comprising an active 3-HP pathway and a heterologous polynucleotide encoding an aminotransferase), wherein the recombinant cell further comprises lipase or esterase activity in a medium under suitable conditions to produce a 3-HP ester; and (b) recovering the 3-HP ester. In another aspect is a method of producing a 3-HP ester, comprising: (a) cultivating a recombinant cell described herein (e.g., a recombinant host cell described herein comprising an active 3-HP pathway and a heterologous polynucleotide encoding an aminotransferase) in a medium under suitable conditions to produce 3-HP; (b) recovering the 3-HP; (c) esterifying the 3-HP under suitable conditions to produce a 3-HP ester; and (d) recovering the 3-HP ester. The recombinant host cells described herein may also be used to produce polymerized 3-HP in vitro or in vivo using techniques known in the art (e.g., Zhou et al Metab. Eng. 2011, 13(6), 777-785; and U.S. Pat. No. 8,030,045; the contents of which are hereby incorporated by reference).

The 3-HP produced by any of the methods described herein may be converted to acrylic acid. Acrylic acid can be produced by the chemical dehydration of 3-HP using techniques known in the art, e.g., heating in the presence of a catalyst (e.g., a solid oxide dehydration catalyst such as titania or alumina).

In one aspect is a method of producing acrylic acid or a salt thereof, comprising: (a) cultivating a recombinant cell described herein (e.g., a recombinant host cell described herein comprising an active 3-HP pathway and a heterologous polynucleotide encoding an aminotransferase) in a medium under suitable conditions to produce 3-HP; (b) recovering the 3-HP; (c) dehydrating the 3-HP under suitable conditions to produce acrylic acid or a salt thereof; and (d) recovering the acrylic acid or salt thereof.

Suitable assays to test for the production of 3-HP, lactate, and derivatives thereof for the methods of production and cells described herein can be performed using methods known in the art. For example, final 3-HP product and intermediates (e.g., pyruvate), as well as other organic compounds, can be analyzed by methods such as HPLC (High Performance Liquid Chromatography), GC-MS (Gas Chromatography Mass Spectroscopy) and LC-MS (Liquid Chromatography-Mass Spectroscopy) or other suitable analytical methods using routine procedures well known in the art. The release of 3-HP in the fermentation broth can also be tested with the culture supernatant. Byproducts and residual sugar in the fermentation medium (e.g., glucose) can be quantified by HPLC using, for example, a refractive index detector for glucose and alcohols, and a UV detector for organic acids (Lin et al., Biotechnol. Bioeng. 90:775-779 (2005)), or using other suitable assay and detection methods well known in the art.

The invention may be further described by the following numbered paragraphs:

Paragraph [1]. A recombinant yeast cell comprising an active 3-HP pathway capable of producing 3-HP and a heterologous polynucleotide encoding an aminotransferase of E.C. 2.6.1.19, wherein:

(a) the aminotransferase comprises the motif (A,C)-Xn-(GSLS)-Xn-SKA(L/l)(L/l);

(b) the aminotransferase has β-alanine aminotransferase (BAAT) activity; and

(c) the aminotransferase is other than a S. kluyveri, I. orientalis, or S. avermitilis aminotransferase.

Paragraph [2]. The recombinant cell of Paragraph [1], wherein the aminotransferase comprises:

(i) AC at a positions corresponding to positions 103-104 of SEQ ID NO: 20;

(ii) GSLS at positions corresponding to positions 194-197 of SEQ ID NO: 20; and

(iii) SKA(L/l)(L/l) at positions corresponding to positions 354-358 of SEQ ID NO: 20.

Paragraph [3]. A recombinant yeast cell comprising an active 3-HP pathway and a heterologous polynucleotide encoding a β-alanine aminotransferase (BAAT), wherein the BAAT has at least 60% e.g., at least 65%, 70%, 75%, 80%, 85%, 90%, 95%, 97%, 98%, 99%, or 100% sequence identity to SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234; and wherein the cell capable of producing 3-HP.
Paragraph [4]. The recombinant cell of Paragraph [3], wherein the β-alanine aminotransferase (BAAT) has at least 60% e.g., at least 65%, 70%, 75%, 80%, 85%, 90%, 95%, 97%, 98%, 99%, or 100% sequence identity to SEQ ID NO: 206, SEQ ID NO: 207, or SEQ ID NO: 208.
Paragraph [5]. The recombinant cell of Paragraph [3] or [4], wherein the β-alanine aminotransferase (BAAT) comprises the motif (A,C)-Xn-(GSLS)-Xn-SKA(L/l)(L/l).
Paragraph [6]. The recombinant cell of Paragraph [5], wherein the β-alanine aminotransferase (BAAT) comprises:

(i) AC at a positions corresponding to positions 103-104 of SEQ ID NO: 20;

(ii) GSLS at positions corresponding to positions 194-197 of SEQ ID NO: 20; and

(iii) SKA(L/l)(L/l) at positions corresponding to positions 354-358 of SEQ ID NO: 20.

Paragraph [7]. The recombinant cell of any one of Paragraphs [3]-[6], wherein β-alanine aminotransferase (BAAT) differs by no more than ten amino acids, e.g., by no more than five amino acids, by no more than four amino acids, by no more than three amino acids, by no more than two amino acids, or by one amino acid from SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234.
Paragraph [8]. The recombinant cell of any one of Paragraphs [3]-[7], wherein β-alanine aminotransferase (BAAT) differs by no more than ten amino acids, e.g., by no more than five amino acids, by no more than four amino acids, by no more than three amino acids, by no more than two amino acids, or by one amino acid from SEQ ID NO: 206, SEQ ID NO: 207, or SEQ ID NO: 208.
Paragraph [9]. The recombinant cell of any one of Paragraphs [3]-[8], wherein the β-alanine aminotransferase (BAAT) has an amino acid sequence comprising or consisting of SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234.
Paragraph [10]. The recombinant cell of any one of Paragraphs [3]-[9], wherein the β-alanine aminotransferase (BAAT) has an amino acid sequence comprising or consisting of SEQ ID NO: 206, SEQ ID NO: 207, or SEQ ID NO: 208.
Paragraph [11]. The recombinant cell of any one of Paragraphs [3]-[10], wherein the heterologous polynucleotide comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251.
Paragraph [12]. The recombinant cell of any one of Paragraphs [3]-[11], wherein the heterologous polynucleotide comprises a coding sequence having at least 60%, e.g., at least 65%, at least 70%, at least 75%, at least 80%, at least 85%, at least 90%, at least 95%, at least 96%, at least 97%, at least 98%, at least 99%, or 100% sequence identity to SEQ ID NO: 239, SEQ ID NO: 240, or SEQ ID NO: 241.
Paragraph [13]. The recombinant cell of any one of Paragraphs [3]-[12], wherein the heterologous polynucleotide has a coding sequence that comprises or consists of SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251.
Paragraph [14]. The recombinant cell of any one of Paragraphs [3]-[13], wherein the heterologous polynucleotide has a coding sequence that comprises or consists of SEQ ID NO: 239, SEQ ID NO: 240, or SEQ ID NO: 241.
Paragraph [15]. The recombinant cell of any one of Paragraphs [3]-[14], wherein the heterologous polynucleotide comprises a coding sequence that hybridizes under at least low stringency conditions e.g., medium stringency conditions, medium-high stringency conditions, high stringency conditions, or very high stringency conditions with the full-length complementary strand of SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251.
Paragraph [16]. The recombinant cell of any one of Paragraphs [3]-[15], wherein the heterologous polynucleotide comprises a coding sequence that hybridizes under at least low stringency conditions e.g., medium stringency conditions, medium-high stringency conditions, high stringency conditions, or very high stringency conditions with the full-length complementary strand of SEQ ID NO: 239, SEQ ID NO: 240, or SEQ ID NO: 241.
Paragraph [17]. The recombinant cell of any one of Paragraphs [1]-[16], wherein the aminotransferase lacks an N-terminal mitochondrial target sequence (MTS).
Paragraph [18]. The recombinant cell of any one of Paragraphs [1]-[17], wherein the active 3-HP pathway proceeds through a β-alanine and/or malonate semialdehyde intermediate.
Paragraph [19]. The recombinant yeast cell of any one of Paragraphs [1]-[18], wherein the heterologous polynucleotide encoding the β-alanine aminotransferase (BAAT) comprises a promoter foreign to the polynucleotide encoding the β-alanine aminotransferase (BAAT).
Paragraph [20]. The recombinant yeast cell of any of paragraphs [1]-[19], wherein the cell produces a greater amount of 3-HP compared to the cell without the heterologous polynucleotide encoding the β-alanine aminotransferase (BAAT), when cultivated under identical conditions (e.g., at least 10%, at least 15%, at least 20%, at least 25%, at least 30%, at least 35%, at least 40%, at least 45%, at least 50%, at least 75%, at least 100%, at least 150%, at least 200%, at least 250%, at least 300%, or at least 500% more).
Paragraph [21]. The recombinant yeast cell of any one of Paragraphs [1]-[20], wherein the cell produces a greater amount of 3-HP (e.g., at least 10%, at least 15%, at least 20%, at least 25%, at least 30%, at least 35%, at least 40%, at least 45%, at least 50%, at least 75%, at least 100%, at least 200%, at least 300%, or at 500% more) compared to a second recombinant yeast cell, when cultivated under the same conditions; wherein the second cell is identical to the yeast cell with the proviso that the second cell encodes the BAAT of SEQ ID NO: 20 in place of the yeast cell aminotransterase.
Paragraph [22]. The recombinant yeast cell of any of paragraphs [1]-[21], wherein the cell further comprises one or more (e.g., two, several) heterologous polynucleotides selected from: a heterologous polynucleotide encoding a PYC; a heterologous polynucleotide encoding an AAT; a heterologous polynucleotide encoding an ADC; a heterologous polynucleotide encoding a 3-HPDH; and a heterologous polynucleotide encoding a PPC.
Paragraph [23]. The recombinant cell of any one of Paragraphs [1]-[22], wherein the cell is Crabtree-negative or Crabtree-positive.
Paragraph [24]. The recombinant cell of any one of Paragraphs [1]-[23], wherein the cell belongs to a genus selected from Candida, Kluyveromyces, Pichia, Issatchenkia, Schizosaccharomyces, Torulaspora, Zygosaccharomyces, and Saccharomyces.
Paragraph [25]. The recombinant cell of Paragraph [24], wherein the cell belongs to a clade selected from the Saccharomyces clade and the I. orientalis/P. fermentans clade.
Paragraph [26]. The recombinant cell of Paragraph [24], wherein the cell is selected from I. orientalis, C. lambica, and S. bulderi.
Paragraph [27]. The recombinant cell of any one of Paragraphs [1]-[23], wherein the cell is a CNB1 yeast cell.
Paragraph [28]. The recombinant cell of any one of Paragraphs [1]-[26], wherein the yeast cell is unable to ferment pentose sugars.
Paragraph [29]. The recombinant cell of any one of Paragraphs [1]-[28], wherein said cell further comprises a disruption to one or more endogenous dse2, scw11, eaf3, sed1, or sam2 genes, and/or one or more endogenous genes encoding a PDC, ADH, GAL6, CYB2A, CYB2B, GPD, GPP, ALD, or PCK.
Paragraph [30]. The recombinant cell of any one of Paragraphs [1]-[29], wherein said cell further comprises a disruption to an endogenous gene encoding a PDC.
Paragraph [31]. The recombinant cell of any one of Paragraphs [1]-[30], wherein said cell further comprises a disruption to an endogenous gene encoding a GPD.
Paragraph [32]. A composition comprising the recombinant cell of any one of Paragraphs [1]-[31].
Paragraph [33]. The composition of Paragraph [32], wherein the composition comprises a fermentable medium.
Paragraph [34]. The composition of Paragraph [33], wherein the fermentable medium comprises sucrose, glucose, and/or fructose.
Paragraph [35]. The composition of any one of Paragraph [33] or [34], wherein the fermentable medium comprises less than 1% pentose sugars.
Paragraph [36]. The composition of any one of Paragraphs [32]-[35], further comprising 3-HP.
Paragraph [37]. The composition of Paragraph [36], wherein the 3-HP is at a titer greater than about 1 g/L, e.g., greater than about 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
Paragraph [38]. The composition of any one of Paragraphs [32]-[37], wherein the medium is at a pH of less than 6.5, e.g., in the range of about 1.5 to about 4.5, about 2.0 to about 4.0, or about 2.0 to about 3.5.
Paragraph [39]. A method of producing 3-HP, comprising:

(a) cultivating the recombinant yeast cell of any one of Paragraphs [1]-[31] in a fermentable medium under suitable conditions to produce 3-HP; and

(b) recovering the 3-HP.

Paragraph [40]. The method of Paragraph [39], wherein the resulting 3-HP is substantially pure.
Paragraph [41]. A method of producing acrylic acid or a salt thereof, comprising:

(a) cultivating the recombinant cell of any one of Paragraphs [1]-[31] in a fermentable medium under suitable conditions to produce 3-HP;

(b) recovering the 3-HP;

(c) dehydrating the 3-HP under suitable conditions to produce acrylic acid or a salt thereof; and

(d) recovering the acrylic acid or salt thereof.

Paragraph [42]. A method of producing a 3-HP ester, comprising:

(a) cultivating the recombinant cell of any one of Paragraphs [1]-[31] in a fermentable medium under suitable conditions to produce 3-HP;

(b) recovering the 3-HP;

(c) esterifying the 3-HP under suitable conditions to produce a 3-HP ester; and

(d) recovering the 3-HP ester

Paragraph [43]. A method of producing 1,3-propanediol, comprising:

(a) cultivating the recombinant cell of any one of Paragraphs [1]-[31] in a fermentable medium under suitable conditions to produce 3-HP;

(b) recovering the 3-HP;

(c) hydrogenating the 3-HP under suitable conditions to produce 1,3-propanediol; and

(d) recovering the 1,3-propanediol.

Paragraph [44]. The method of any one of Paragraph [39]-[43], wherein the fermentable medium comprises sucrose, glucose, and/or fructose.
Paragraph [45]. The method of any one of Paragraph [39]-[44], wherein the fermentable medium comprises less than 1% pentose sugars.
Paragraph [46]. The method of any one of Paragraphs [39]-[45], wherein the fermentable medium is at a pH of less than 6.5, e.g., in the range of about 1.5 to about 4.5, about 2.0 to about 4.0, or about 2.0 to about 3.5.
Paragraph [47]. The method of any one of Paragraphs [39]-[46], wherein the produced 3-HP is at a titer greater than about 1 g/L, e.g., greater than about 2 g/L, 5 g/L, 10 g/L, 15 g/L, 20 g/L, 25 g/L, 30 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 125 g/L, 150 g/L, 200 g/L, or 250 g/L.
Paragraph [48]. The method of any one of Paragraphs [39]-[47] wherein the recombinant cell is a CNB1 cell cultivated in a fermentable medium comprising less than 1% pentose sugars.

The following examples are provided by way of illustration and are not intended to be limiting of the invention.

### EXAMPLES

Chemicals used as buffers and substrates were commercial products of at least reagent grade.

### Strains

The strain MeJi412 was constructed from I. orientalis CD1822 as described in WO 2012/074818 (the content of which is incorporated herein by reference) and expresses an ADC gene to provide an active 3-HP pathway that proceeds through a β-alanine intermediate.

The strain McTs236 was constructed from MeJi412 as described in WO 2012/074818 (the content of which is incorporated herein by reference) and contains a homozygous deletion of the native gene encoding the I. orientalis BAAT (pyd4) of SEQ ID NO: 20.

### Media and Solutions

2× YT+amp plates were composed of 16 g/L tryptone, 10 g/L yeast extract, 5 g/L NaCl, 100 mg/L ampicillin, and 15 g/L Bacto agar.

Yeast SD-ura selection plates were composed of 6.7 g yeast nitrogen base with ammonium sulfate, 1 g yeast synthetic dropout supplement w/o uracil 20 g bacteriological agar, and deionized water to a final volume of 960 mL. Following autoclave sterilization, 40 mL sterile 50% glucose was added and plates poured.

Yeast SD-ura selection media was composed of 6.7 g yeast nitrogen base with ammonium sulfate, 1 g yeast synthetic dropout supplement w/o uracil 20 g bacteriological agar, and deionized water to a final volume of 960 mL. Following autoclave sterilization, 40 mL sterile 50% glucose was added and plates poured.

YP+10% glucose media was composed of 500 mL YP broth and 100 mL sterile 50% glucose.

YP broth was composed of 10 g/L of yeast extract, 20 g/L of peptone.

TBE was composed of 10.8 g/L of Tris base, 5.5 g/L boric acid, and 4 mL/L of 0.5 M EDTA pH 8.0.

LiOAc/TE solution was composed of 8 parts sterile water, 1 part 1 M LiOAc, and 1 part 10× TE.

10× TE (200 mL) was composed of 2.42 g Tris Base, 4 mL 0.5M EDTA, pH 8.0. 5 M HCl was used to adjust the pH to 7.5 and the solution was sterilized by autoclave.

PEG/LiOAc/TE Solution was composed of 8 parts 50% PEG3350, 1 part 1 M LiOAc, and 1 part 10× TE.

50% PEG3350 was prepared by adding 100 g PEG3350 to 150 mL water and heating and stirring until dissolved. The volume was then brought up to 200 mL with water and the sterilized by autoclave.

Trace element solution was composed of EDTA (15.0 g/L), zinc sulfate heptahydrate (4.5 g/L), manganese chloride dehydrate (1.0 g/L), Cobalt(II)chloride hexahydrate (0.3 g/L), Copper(II)sulfate pentahydrate (0.3 g/L), disodium molybdenum dehydrate (0.4 g/L), calcium chloride dehydrate (4.5 g/L), iron sulphate heptahydrate (3 g/L), boric acid (1.0 g/L), and potassium iodide (0.1 g/L).

Vitamin solution was composed of biotin (D-; 14.1 g/L), calcium pantothenate (D+; 1 g/L), nicotinic acid (5 g/L), myo-inositol (25 g/L), pyridoxine hydrochloride (1 g/L), p-aminobenzoic acid (0.2 g/L), and thiamine hydrochloride (1 g/L).

CNB1 shake flask media was composed of urea (2.3 g/L), magnesium sulfate heptahydrate (0.5 g/L), potassium phosphate monobasic (3.0 g/L), trace element solution (1 mL/L) and vitamin solution (1 mL/L), glucose (120.0 g/L), 2-(N-Morpholino)ethanesulfonic acid (MES) (97.6 g/L). After dissolving all medium components, the pH of the medium was adjusted to an initial pH of 5.8 using an appropriate base (e.g., KOH).

TABLE 1
Primers sequences used in the Examples.

| Identi- | SEQ ID | |
|---|---|---|
| fier | NO: | Sequence (5′-3′) |
| 611245 | 255 | CGAACCAATTCAAGAAAACCAAC |
| 611317 | 256 | GGTCCTTACATCTGTCTAGT |
| 611678 | 257 | TTGTTGCCATCTGCATCCAC |
| 611717 | 258 | CGCTACGATACGCTACGATA |
| 611815 | 259 | CTCCTTCATTTAACTATACCAGACG |
| 612794 | 260 | GAAGGGGGTCCAAGTTATCC |
| 612795 | 261 | CCAACAATCTTAATTGGTGAC |
| 612908 | 262 | GATATGGGCGGTAGAGAAGA |
| 612909 | 263 | GCTCCTTCAAAGGCAACACA |
| 612910 | 264 | TGAACTATCACATGAACGTA |
| 612911 | 265 | TCAAGGTAGGGTCACTTAAC |
| 613242 | 266 | TCCATAATCCAATAGATCATCCC |
| 613243 | 267 | AACACAATGGAACCAACCTAGT |
| 614179 | 268 | CGCATACATTATGGTAGATG |
| 614180 | 269 | GATTGAAGATTCTTACAGGC |
| 614626 | 270 | AGGGTACCTTAGTACGAAGG |
| 614627 | 271 | CTATTCTTACGATGAAGGCG |
| 614851 | 272 | TAGTTAATTAATTACAGAGTTGGTAAGACTT |
| 614852 | 273 | GACTCTAGATAAAATGTCTTACGCTTCAAGATA |
| 614945 | 274 | GTCAGACTTTGCAACCTC |
| 1212900 | 275 | ATGTAAACGTCCAACAACTTG |
| 1212904 | 276 | GTACTTCCCAAGAGAGCCAT |
| 1213764 | 277 | GATGTCGTAGTAGTCGGTAG |
| 1213770 | 278 | CGAAGCAAACAACTTGGTTG |
| 1213862 | 279 | ATGCCAATGCTGGTCTGTTTG |

### Example 1: Procedure for DNA Transformation

DNA transformation into the host genome to generate the recombinant strains described in the following examples was conducted based on the specific procedure below.

Three mL of YP+10% glucose media was added to a 14 mL Falcon tube and the desired strain was inoculated into this media using a sterile loop. The culture was grown with shaking at 250 rpm overnight (˜16 hr) at 37° C. 0.5 mL of the overnight culture was added to a 125 mL baffled flask containing 25 mL of liquid YP+10% glucose media. The flask was grown with shaking at 250 rpm at 37° C. Small aliquots of the culture were withdrawn at approximately hourly intervals and the OD₆₀₀ was measured. The culture was grown until the OD₆₀₀ was 0.6-1.0.

The cells were harvested by centrifugation at 2279×g at room temperature, the pellet was resuspended in 25 mL sterile water, then centrifuged at 2279×g at room temperature. The pellet was resuspended in 1 mL sterile water, and the resuspended cells were transferred to a 1.5 mL tube and then pelleted at 16,100×g. The cells were resuspended in 1 mL LiOAc/TE solution and then pelleted at 16,100×g. The cell pellet was then resuspended in 250 μL LiOAc/TE solution.

The following components were added to a 1.5 mL tube: 100 μL of the above cells, 10 μL freshly boiled then iced salmon sperm DNA (Agilent Technologies, Santa Clara, Calif., USA), and 10 μL of the desired, linearized transforming DNA. A control reaction with water instead of DNA was also prepared. To each transformation reaction, 600 μL of PEG/LiOAc/TE Solution was added, followed by 40 μL DMSO and the reactions were inverted several times to mix. The transformation reactions were incubated in a 42° C. water bath for 15 minutes, and cells were pelleted at 5,400×g for 1 min. Cells were resuspended in water, split in two, and each half of the transformation reaction was plated to a yeast SD-ura selection plate. Plates were placed at 37° C. Colonies were visible after 2 days of growth.

### Example 2: Identification and Analysis of Aminotransferases

Sequences from approximately 50 BAAT and gabT aminotransferase homologs were analyzed for regions that may be key for specific activity toward the β-alanine or GABA substrates. Based on alignment (e.g., FIG. 2) three putative conserved regions were identified as important for specificity of β-alanine: AC at positions 103-104, GSLS at positions 194-197, and SKA(L/l)(L/l) at positions 354-358 (corresponding to SEQ ID NO: 20). A representative summary for aminotransferases of numerous species is shown in Table 2.

For genomes with two aminotransferase homologs, we observed that the homologs with an N-terminal mitochondrial target sequence (MTS) resemble BAAT by containing the three regions of interest. At positions 103-104 the amino acids were AC for the MTS homologs and GN for the non-MTS homologs from those same organisms. At the 194-197 region, the mitochondrial homologs were usually GSLS and the non-MTS homologs were usually ASGS. Lastly, positions 354-358 for the mitochondrial homologs were conserved SKA with either an isoleucine or leucine. In the non-MTS homologs, the sequence was primarily ARMIL. Both S. kluyveri and I. orientalis contained BAAT and gabT homologs (all without MTS).

For genomes with only one identified aminotransferase homolog, C. arabinofermentans, P. membranifaciens, and B. bruxellensis all show putative non-MTS BAAT homologs.

TABLE 2

| | | | Assingment: BAAT | | | Assingment: gabT | | |
|---|---|---|---|---|---|---|---|---|
| Organism | SEQ ID | MTS | 103-104 | 194-197 | 354-358 | 103-104 | 194-197 | 354-358 |
| S. kluyveri | 21 | no | AC | GSLS | SKALI | | | |
| | 212 | no | | | | GN | ASGS | ARMII |
| I. orientalis | 20 | no | AC | GSLS | SKALI | | | |
| | 211 | no | | | | GN | ASGS | KGVIL |
| S. pombe | 213 | no | | | | GN | GSLS | FRAVQ |
| S. cerevisiae | 24 | no | | | | GN | ASGS | ARMII |
| K. lactis | 214 | no | | | | GN | ASGS | ARMII |
| N. castelli | 216 | no | | | | GN | ASGS | ARILI |
| A. gossypii | 217 | no | | | | GN | ASGS | AHMII |
| C. parapsilosis | 218 | yes | AC | GSLS | SKAIL | | | |
| | 219 | no | | | | GN | ASGS | ARMTL |
| S. stipitis | 220 | yes | AC | GSLS | SKAII | | | |
| | 221 | no | | | | GN | ASGS | ARMIL |
| L. elongisporius | 222 | yes | AC | GALA | SKALI | | | |
| | 223 | no | | | | GN | ASGS | ARMIL |
| C. guilliermondii | 209 | yes | AC | GSLS | SKALI | | | |
| | 210 | no | | | | GN | ASGS | ARMIL |
| C. lusitaniae | 224 | yes | AC | GSLS | SKALI | | | |
| | 225 | no | | | | GN | ASAS | ARMIL |
| C. albicans | 226 | yes | AC | GSLS | SKALI | | | |
| | 227 | no | | | | GN | ASGS | ARMIL |
| D. hansenii | 228 | yes | AC | GSLS | SKALI | | | |
| | 229 | no | | | | GN | ASGS | ARMIL |
| C. tropicalis | 230 | yes | AC | GSLS | SKAIL | | | |
| | 231 | no | | | | GN | ASGS | ARMIL |
| K. capsulata | 232 | yes | AC | GSLS | SKALI | | | |
| | 233 | no | | | | GN | ATAS | TKMII |
| M. farinosa | 234 | yes | AC | GSLS | SKALL | | | |
| | 235 | no | | | | GN | ASGS | ARMIL |
| C. arabinofermentans | 207 | no | AC | GSLS | SKALI | | | |
| P. membranifaciens | 206 | no | AC | GSLS | SKALI | | | |
| A. niger | 236 | yes | | | | GN | GSLS | ARALI |
| B. bruxellensis | 208 | no | AC | GSLS | SKALI | | | |

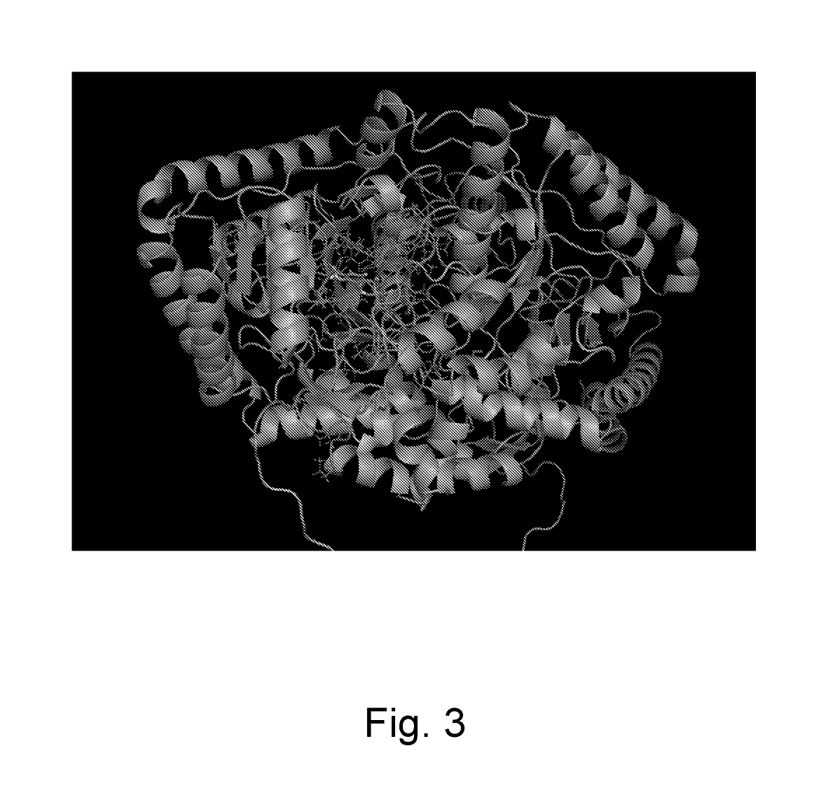

The significance of the motif (A,C)-Xn-(GSLS)-Xn-SKA(L/l)(L/l) for BAAT activity can be identified rough molecular modeling, wherein the 103-104 region was determined to be at the entrance of the enzyme active site and the 194-197 region sits near the active site in the PLP binding area (FIG. 3). The 354-358 region appears to be near the surface of the protein (FIG. 4).

### Example 3: Construction of an Insertion Vector for Expressing a Heterologous Candida arabinofermentans Aminotransferase at the Adh9091 Locus (pMcTs343)

The plasmid pMcTs343 was designed to allow integration of a coding sequence of a Candida arabinofermentans aminotransferase at the adh9091 locus under the control of the PDC promoter and terminator using URA3 as a selectable marker, and was constructed as described below.

A codon-optimized version of an aminotransferase coding sequence from C. arabinofermentans (SEQ ID NO: 240) and flanked by 5′ XbaI site and 3′ PacI restriction sites, was provided by GeneArt (Life Technologies Corporation) in a DNA String fragment designated 14ACKV6P. This fragment was cloned into pCR-BluntII-TOPO vector (Invitrogen) and transformed into Stellar chemically competent cells (Clontech) according to manufacturer's instructions. After a recovery period, two 100 μl aliquots from the transformation mixture were plated onto 150 mm 2×YT plates supplemented with 50 μg of kanamycin per ml and incubated overnight at 37° C. Recombinant colonies of the transformations were each inoculated into 3 ml of LB medium supplemented with kanamycin (50 μg/ml). Plasmid DNA was prepared from these cultures using a BIOROBOT® Universal System (Qiagen, Inc.) and analyzed by restriction digestion and sequencing. The plasmid DNA from one clone having the correct restriction digest pattern for C. arabinofermentans aminotransferase coding sequence in PCR II-Blunt-TOPO vector was designated pMcTs338.

Plasmid pMcTs338 was digested with XbaI and PacI and the 1.45 kbp fragment containing the C. arabinofermentans aminotransferase coding sequence was gel isolated and purified NucleoSpin Gel and PCR clean up kit (Macherey-Nagel).

Plasmid pMBin204 (described in WO2012074818) was digested with XbaI and PacI and the 8.4 kbp fragment was gel isolated and purified using the NucleoSpin Gel and PCR clean up kit (Macherey-Nagel).

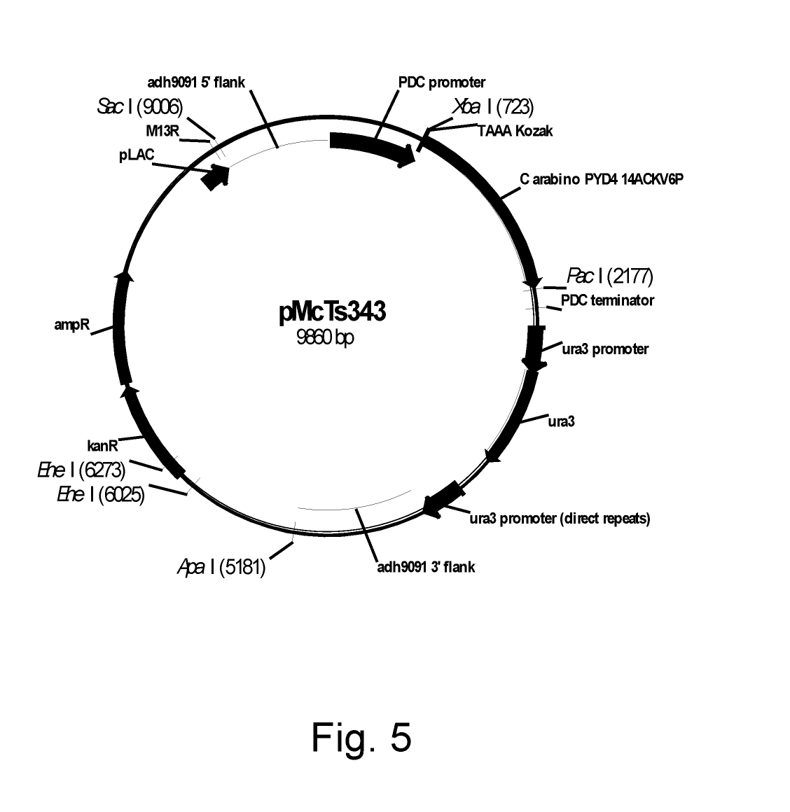

Cloning of the DNA fragment containing the coding sequence of the C. arabinofermentans aminotransferase (supra) into the 8.4 kbp pMBin204 linearized vector (supra) was performed using T4 DNA ligase (New England Biolabs). The reaction mixture contained 1× T4 DNA ligase buffer, 1 μl T4 DNA ligase, 1 μl of the pMBin204 XbaI/PacI digested DNA fragment above, and 5 μl of the pMcTs338 XbaI/PacI digested product above in total volume of 20 μl. The reaction mixture was incubated at room temperature for at least 1 hour and subsequently used to transform Stellar Competent Cells (Clontech) according to manufacturer's instructions. After a recovery period, two 100 μl aliquots from the transformation mixture were plated onto 150 mm 2×YT plates supplemented with 100 μg of ampicillin per ml and incubated overnight at 37° C. Recombinant colonies of the transformations were each inoculated into 3 ml of LB medium supplemented with ampicillin (100 μg/ml). Plasmid DNA was prepared from these cultures using a BIOROBOT® Universal System (QIAGEN Inc.) and subjected to restriction digest checks. The plasmid DNA from one clone having the correct restriction digest pattern was further subjected to sequence analysis and designated pMcTs343 (FIG. 5).

Plasmid pMcTs343 contains a C. arabinofermentans aminotransferase coding sequence (SEQ ID NO: 240, which encodes for SEQ ID NO: 207) under control of the PDC promoter and terminator, with the URA3 selectable marker flanked by regions of homology to the adh9091 locus. The aminotransferase of SEQ ID NO: 207 is identified (based on the analysis supra) as a β-alanine aminotransferase (BAAT) and comprises the motif (A,C)-Xn-(GSLS)-Xn-SKA(L/l)(L/l), specifically, (i) AC at a positions corresponding to positions 103-104 of SEQ ID NO: 20; (ii) GSLS at positions corresponding to positions 194-197 of SEQ ID NO: 20; and (iii) SKALI at positions corresponding to positions 354-358 of SEQ ID NO: 20 (See, Table 2 and FIG. 2).

### Example 4: Construction of an Insertion Vector for Expressing a Heterologous Brettanomyces bruxellensis Aminotransferase at the Adh9091 Locus (pMcTs351)

The plasmid pMcTs351 was designed to allow integration of a coding sequence of a Brettanomyces bruxellensis aminotransferase at the adh9091 locus under the control of the PDC promoter and terminator using URA3 as a selectable marker, and was constructed as described below.

A codon-optimized version of an aminotransferase coding sequence from B. bruxellensis (SEQ ID NO: 241) and flanked by 5′ XbaI site and 3′ PacI restriction sites, was provided by GeneArt (Life Technologies Corporation) in a DNA String fragment designated 14AC24TP.

Plasmid pMBin204 (described in WO2012074818) was digested with XbaI and PacI and the 8.4 kbp fragment was gel isolated and purified using the NucleoSpin Gel and PCR clean up kit (Macherey-Nagel).

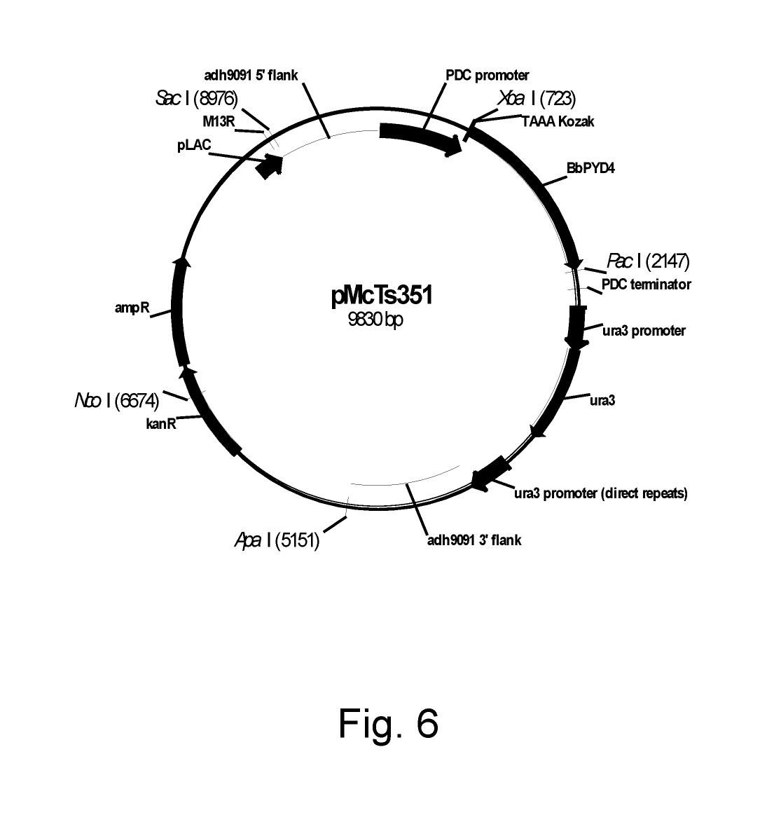

The DNA fragment 14AC24TP (supra) was cloned into the pMBin204 XbaI/PacI digested DNA fragment using InFusion HD (Clontech) and transformed into Stellar (Clontech) chemically competent cells according to manufacturer's instructions. After a recovery period, two 100 μl aliquots from the transformation mixture were plated onto 150 mm 2×YT plates supplemented with 100 μg of ampicillin per ml and incubated overnight at 37° C. Recombinant colonies of the transformations were each inoculated into 3 ml of LB medium supplemented with ampicillin (100 μg/ml). Plasmid DNA was prepared from these cultures using a BIOROBOT® Universal System (QIAGEN Inc.) and subjected restriction digest checks. The plasmid DNA from one clone having the correct restriction digest pattern was further subjected to sequence analysis and designated pMcTs351 (FIG. 6).

Plasmid pMcTs351 contains a B. bruxellensis aminotransferase coding sequence (SEQ ID NO: 241, which encodes for SEQ ID NO: 208) under control of the PDC promoter and terminator, with the URA3 selectable marker flanked by regions of homology to the adh9091 locus. The aminotransferase of SEQ ID NO: 208 is identified (based on the analysis supra) as a β-alanine aminotransferase (BAAT) and comprises the motif (A,C)-Xn-(GSLS)-Xn-SKA(L/l)(L/l), specifically, (i) AC at a positions corresponding to positions 103-104 of SEQ ID NO: 20; (ii) GSLS at positions corresponding to positions 194-197 of SEQ ID NO: 20; and (iii) SKALI at positions corresponding to positions 354-358 of SEQ ID NO: 20 (See, Table 2 and FIG. 2).

### Example 5: Construction of an Insertion Vector for Expressing a Heterologous Pichia membranifaciens Aminotransferase at the Adh9091 Locus (pMcTs352)

The plasmid pMcTs352 was designed to allow integration of a coding sequence of a Pichia membranifaciens aminotransferase at the adh9091 locus under the control of the PDC promoter and terminator using URA3 as a selectable marker, and was constructed as described below.

A codon-optimized version of an aminotransferase coding sequence from P. membranifaciens (SEQ ID NO: 239) and flanked by 5′ XbaI site and 3′ PacI restriction sites, was provided by GeneArt (Life Technologies Corporation) in a DNA String fragment designated 14ACYSOP.

Plasmid pMBin204 (described in WO2012074818) was digested with XbaI and PacI and the 8.4 kbp fragment was gel isolated and purified using the NucleoSpin Gel and PCR clean up kit (Macherey-Nagel).

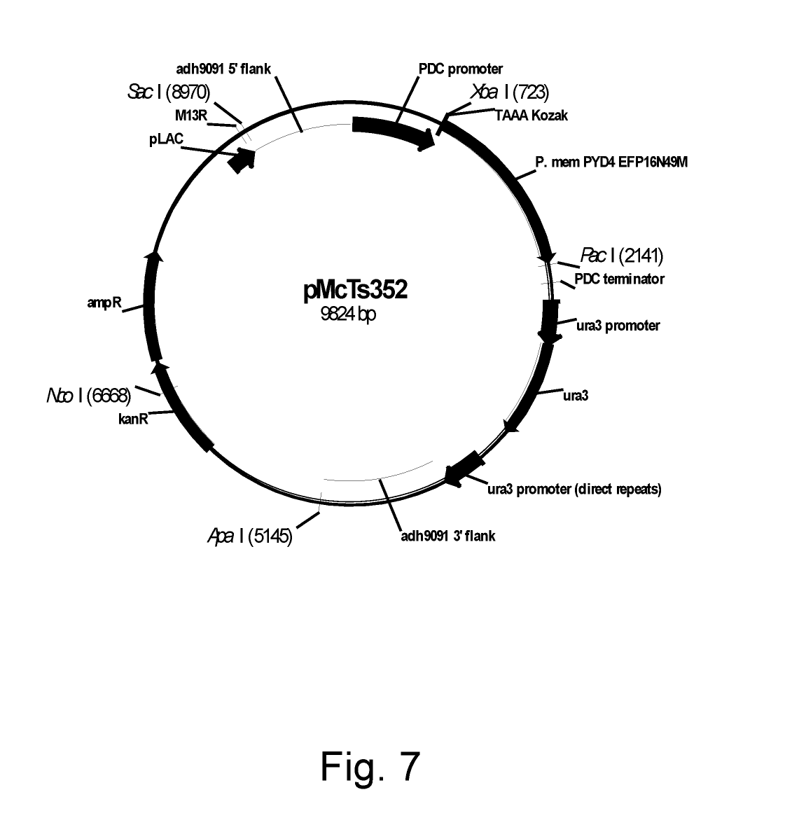

The DNA fragment 14ACYSOP (supra) was cloned into the pMBin204 XbaI/PacI digested DNA fragment using InFusion HD (Clontech) and transformed into Stellar (Clontech) chemically competent cells according to manufacturer's instructions. After a recovery period, two 100 μl aliquots from the transformation mixture were plated onto 150 mm 2×YT plates supplemented with 100 μg of ampicillin per ml and incubated overnight at 37° C. Recombinant colonies of the transformations were each inoculated into 3 ml of LB medium supplemented with ampicillin (100 μg/ml). Plasmid DNA was prepared from these cultures using a BIOROBOT® Universal System (QIAGEN Inc.) and subjected restriction digest checks. The plasmid DNA from one clone having the correct restriction digest pattern was further subjected to sequence analysis and designated pMcTs352 (FIG. 7).

Plasmid pMcTs352 contains a P. membranifaciens aminotransferase coding sequence (SEQ ID NO: 239, which encodes for SEQ ID NO: 206) under control of the PDC promoter and terminator, with the URA3 selectable marker flanked by regions of homology to the adh9091 locus. The aminotransferase of SEQ ID NO: 206 is identified (based on the analysis supra) as a β-alanine aminotransferase (BAAT) and comprises the motif (A,C)-Xn-(GSLS)-Xn-SKA(L/l)(L/l), specifically, (i) AC at a positions corresponding to positions 103-104 of SEQ ID NO: 20; (ii) GSLS at positions corresponding to positions 194-197 of SEQ ID NO: 20; and (iii) SKALI at positions corresponding to positions 354-358 of SEQ ID NO: 20 (See, Table 2 and FIG. 2).

### Example 6: Construction of an Insertion Vector for Expressing a Heterologous Candida guilliermondii Aminotransferase at the Adh9091 Locus (pMcTs356)

The plasmid pMcTs356 was designed to allow integration of a coding sequence of a Candida guilliermondii aminotransferase at the adh9091 locus under the control of the PDC promoter and terminator using URA3 as a selectable marker, and was constructed as described below.

A codon-optimized version of an aminotransferase coding sequence from C. guilliermondii (SEQ ID NO: 242) and flanked by 5′ XbaI site and 3′ PacI restriction sites, was provided by GeneArt (Life Technologies Corporation) in a DNA String fragment designated 14AHQJPC.

Plasmid pMBin204 (described in WO2012074818) was digested with XbaI and PacI and the 8.4 kbp fragment was gel isolated and purified using the NucleoSpin Gel and PCR clean up kit (Macherey-Nagel).

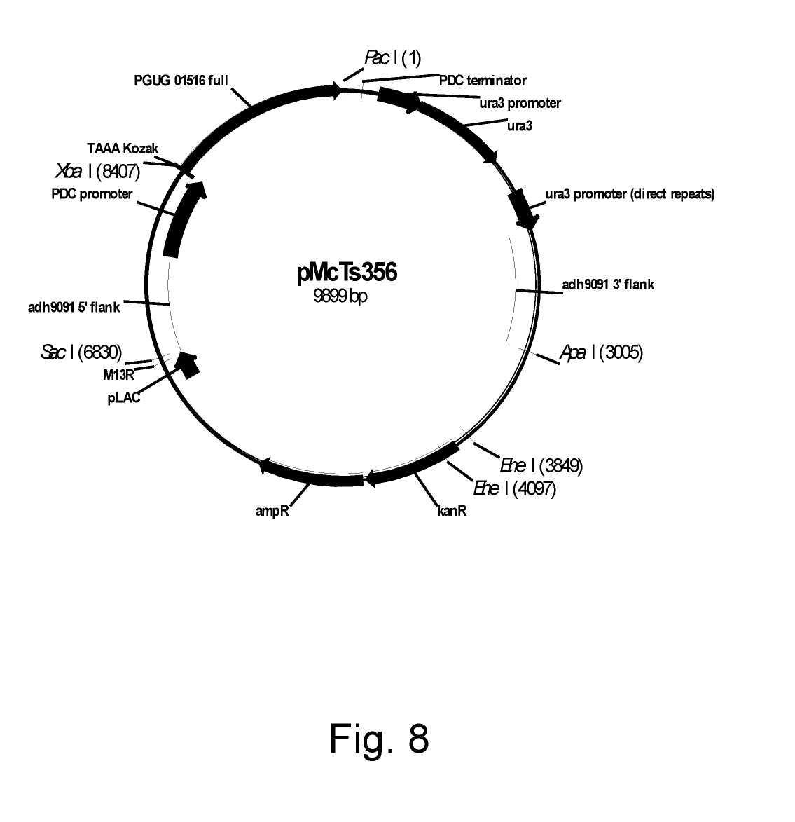

The DNA fragment 14AHQJPC (supra) was cloned into the pMBin204 XbaI/PacI digested DNA fragment using InFusion HD (Clontech) and transformed into Stellar (Clontech) chemically competent cells according to manufacturer's instructions. After a recovery period, two 100 μl aliquots from the transformation mixture were plated onto 150 mm 2×YT plates supplemented with 100 μg of ampicillin per ml and incubated overnight at 37° C. Recombinant colonies of the transformations were each inoculated into 3 ml of LB medium supplemented with ampicillin (100 μg/ml). Plasmid DNA was prepared from these cultures using a BIOROBOT® Universal System (QIAGEN Inc.) and subjected restriction digest checks. The plasmid DNA from one clone having the correct restriction digest pattern was further subjected to sequence analysis and designated pMcTs356 (FIG. 8).

Plasmid pMcTs356 contains a C. guilliermondii aminotransferase coding sequence (SEQ ID NO: 242, which encodes for SEQ ID NO: 209) under control of the PDC promoter and terminator, with the URA3 selectable marker flanked by regions of homology to the adh9091 locus. The aminotransferase of SEQ ID NO: 209 is identified (based on the analysis supra) as a β-alanine aminotransferase (BAAT) and comprises the motif (A,C)-Xn-(GSLS)-Xn-SKA(L/l)(L/l), specifically, (i) AC at a positions corresponding to positions 103-104 of SEQ ID NO: 20; (ii) GSLS at positions corresponding to positions 194-197 of SEQ ID NO: 20; and (iii) SKALI at positions corresponding to positions 354-358 of SEQ ID NO: 20 (See, Table 2).

### Example 7: Construction of an Insertion Vector for Expressing a Heterologous Candida guilliermondii Aminotransferase at the Adh9091 Locus (pMcTs342)

The plasmid pMcTs342 was designed to allow integration of a coding sequence of a Candida guilliermondii aminotransferase at the adh9091 locus under the control of the PDC promoter and terminator using URA3 as a selectable marker, and was constructed as described below.

A codon-optimized version of an aminotransferase coding sequence from C. guilliermondii (SEQ ID NO: 252) and flanked by 5′ XbaI site and 3′ PacI restriction sites, was provided by GeneArt (Life Technologies Corporation) in a DNA String fragment designated 14ACKV7P. This fragment was cloned into pCR-BluntII-TOPO vector (Invitrogen) and transformed into Stellar chemically competent cells (Clontech) according to manufacturer's instructions. After a recovery period, two 100 μl aliquots from the transformation mixture were plated onto 150 mm 2×YT plates supplemented with 50 μg of kanamycin per ml and incubated overnight at 37° C. Recombinant colonies of the transformations were each inoculated into 3 ml of LB medium supplemented with kanamycin (50 μg/ml). Plasmid DNA was prepared from these cultures using a BIOROBOT® Universal System (Qiagen, Inc.) and analyzed by restriction digestion and sequencing. The plasmid DNA from one clone having the correct restriction digest pattern for C. guilliermondii aminotransferase coding sequence in PCR II-Blunt-TOPO vector was designated pMcTs337.

Plasmid pMcTs338 was digested with XbaI and PacI and the 1.4 kbp fragment containing the C. guilliermondii aminotransferase coding sequence was gel isolated and purified NucleoSpin Gel and PCR clean up kit (Macherey-Nagel).

Plasmid pMBin204 (described in WO2012074818) was digested with XbaI and PacI and the 8.4 kbp fragment was gel isolated and purified using the NucleoSpin Gel and PCR clean up kit (Macherey-Nagel).

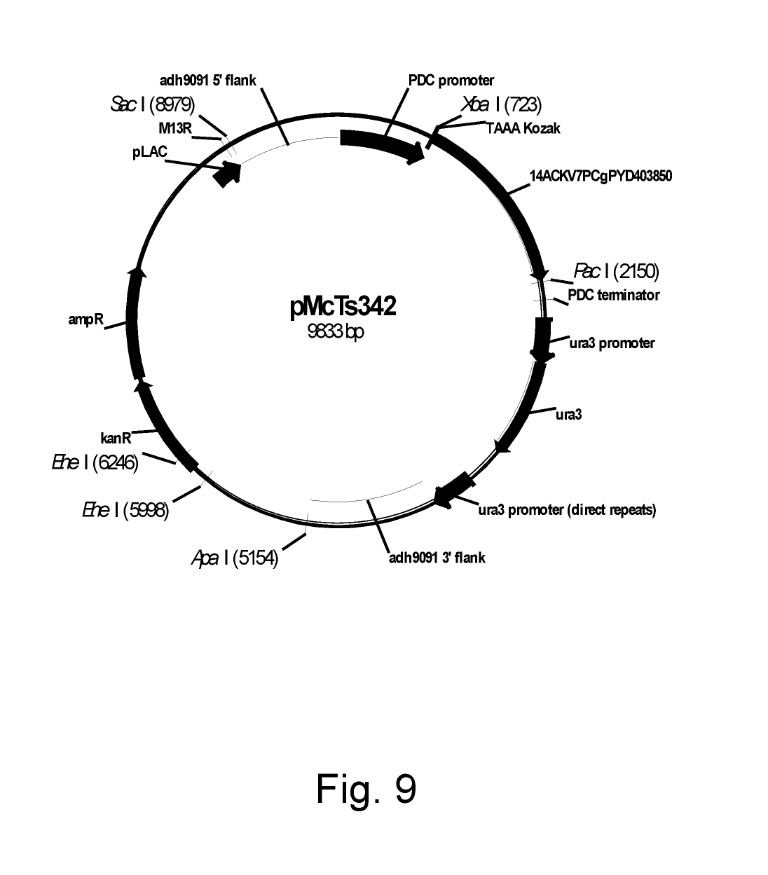

Cloning of the DNA fragment containing the coding sequence of the C. guilliermondii aminotransferase (supra) into the 8.4 kbp pMBin204 linearized vector (supra) was performed using T4 DNA ligase (New England Biolabs). The reaction mixture contained 1× T4 DNA ligase buffer, 1 μl T4 DNA ligase, 1 μl of the pMBin204 XbaI/PacI digested DNA fragment above, and 5 μl of the pMcTs337 XbaI/PacI digested product above in total volume of 20 μl. The reaction mixture was incubated at room temperature for at least 1 hour and subsequently used to transform Stellar Competent Cells (Clontech) according to manufacturer's instructions. After a recovery period, two 100 μl aliquots from the transformation mixture were plated onto 150 mm 2×YT plates supplemented with 100 μg of ampicillin per ml and incubated overnight at 37° C. Recombinant colonies of the transformations were each inoculated into 3 ml of LB medium supplemented with ampicillin (100 μg/ml). Plasmid DNA was prepared from these cultures using a BIOROBOT® Universal System (QIAGEN Inc.) and subjected to restriction digest checks. The plasmid DNA from one clone having the correct restriction digest pattern was further subjected to sequence analysis and designated pMcTs342 (FIG. 9).

Plasmid pMcTs342 contains a C. guilliermondii aminotransferase coding sequence (SEQ ID NO: 252, which encodes for SEQ ID NO: 210) under control of the PDC promoter and terminator, with the URA3 selectable marker flanked by regions of homology to the adh9091 locus. The aminotransferase of SEQ ID NO: 210 is identified (based on the analysis supra) as a 4-aminobutyrate aminotransferase (gabT) as it lacks the motif (A,C)-Xn-(GSLS)-Xn-SKA(L/l)(L/l), specifically, it contains (i) GN at a positions corresponding to positions 103-104 of SEQ ID NO: 20; (ii) ASGS at positions corresponding to positions 194-197 of SEQ ID NO: 20; and (iii) ARMIL at positions corresponding to positions 354-358 of SEQ ID NO: 20 (See, Table 2).

### Example 8: Construction of an Insertion Vector for Expressing a Heterologous I. orientalis Aminotransferase at the Adh9091 Locus (pMBin211)

The plasmid pMBin211 was designed to allow integration of a coding sequence of a I. orientalis aminotransferase at the adh9091 locus under the control of the PDC promoter and terminator using URA3 as a selectable marker, and was constructed as described below.

The I. orientalis aminotransferase coding sequence (SEQ ID NO: 253) was amplified from MBin500 I. orientalis genomic DNA (WO2012074818) using primers 614851 and 614852. During application, the primer 614852 adds a kozak sequence (TAAA) and XbaI restriction site to the 5′ end of the sequence, and primer 614851 adds a Pad restriction site to the 3′ end of the sequence. Fifty pmoles of each primer was used in a PCR reaction containing 50 ng of MBin500 genomic DNA as template, 0.25 mM each dATP, dGTP, dCTP, dTTP, 1× Failsafe Buffer A (Epicentre), and 1.25 units FailSafe PCR Enzyme Mix (Epicentre) in a final volume of 50 μL. The PCR was performed in an EPPENDORF® MASTERCYCLER® (Eppendorf Scientific) programmed for one cycle at 95° C. for 1.5 minutes followed by 35 cycles each at 95° C. for 30 seconds, 55° C. for 30 seconds, and 72° C. for 1.5 minutes with a final extension at 72° C. for 5 minutes. Following thermocycling, the PCR reaction products were cleaned up QIAQUICK® PCR clean up Kit (Qiagen) then digested with XbaI and Pad restriction enzymes. The digests were separated by 1.0% agarose gel electrophoresis in TBE buffer where an approximately 1.4 kbp PCR product was excised from the gel and purified using a using a QIAQUICK® Gel Extraction Kit (Qiagen) according to the manufacturer's instructions.

Plasmid pMBin204 (described in WO2012074818) was digested with XbaI and Pad and the 8.4 kbp fragment was gel isolated and purified using the NucleoSpin Gel and PCR clean up kit (Macherey-Nagel).

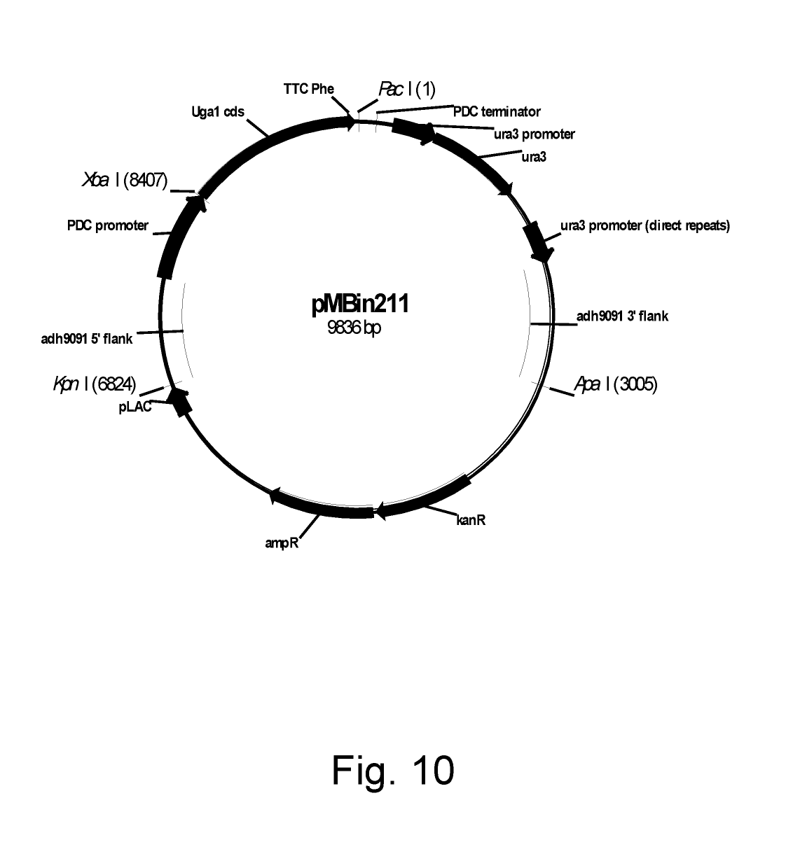

Cloning of the DNA fragment containing the coding sequence of the I. orientalis aminotransferase (supra) into the 8.4 kbp pMBin204 linearized vector (supra) was performed using T4 DNA ligase (New England Biolabs). The reaction mixture contained 1 ul pMBin204 XbaI/PacI DNA fragment, 4 ul of the 1.4 kbp XbaI/PacI PCR product (supra), 1 ul T4 DNA ligase Buffer (New England Biolabs), and 1 ul T4 DNA ligase (New England Biolabs) in a final volume of 10 μl. The reaction was incubated at room temperature overnight then 4 μl was used to transform One Shot TOP10 cells (Invitrogen) according to manufacturer's instructions. The transformations were plated onto 2×YT+kan plates (50 μg/ml) and incubated at 37° C. overnight. Several of the resulting transformants were screened by digestion. Clones yielding the desired insert size were confirmed to be correct by DNA sequencing. One clone containing the correct was designated pMBin211 (FIG. 10).

Plasmid pMBin211 contains an I. orientalis aminotransferase coding sequence (SEQ ID NO: 253, which encodes for SEQ ID NO: 211) under control of the PDC promoter and terminator, with the URA3 selectable marker flanked by regions of homology to the adh9091 locus. The aminotransferase of SEQ ID NO: 211 is identified (based on the analysis supra) as a 4-aminobutyrate aminotransferase (gabT) as it lacks the motif (A,C)-Xn-(GSLS)-Xn-SKA(L/l)(L/l), specifically, it contains (i) GN at a positions corresponding to positions 103-104 of SEQ ID NO: 20; (ii) ASGSV at positions corresponding to positions 194-197 of SEQ ID NO: 20; and (iii) KGVIL at positions corresponding to positions 354-358 of SEQ ID NO: 20 (See, Table 2 and FIG. 2).

### Example 9: Construction of an Insertion Vector for Expressing a Heterologous Saccharomyces kluyveri Aminotransferase at the Adh9091 Locus (pMBin218)

The plasmid pMBin218 was designed to allow integration of a coding sequence of a Saccharomyces kluyveri aminotransferase at the adh9091 locus under the control of the PDC promoter and terminator using URA3 as a selectable marker, and was constructed as described below.

A codon-optimized version of an aminotransferase coding sequence from Saccharomyces kluyveri (SEQ ID NO: 254) and flanked by 5′ XbaI site and 3′ PacI restriction sites, was provided by GeneArt (Life Technologies Corporation) in a plasmid designated 1045171. The plasmid 1045171 was digested with XbaI and PacI and the 1.4 kbp fragment was gel isolated and purified using the QIAQUICK® Gel Extraction Kit (Qiagen) according to the manufacturer's instructions.

Plasmid pMBin204 (described in WO2012074818) was digested with XbaI and PacI and the 8.4 kbp fragment was gel isolated and purified using the NucleoSpin Gel and PCR clean up kit (Macherey-Nagel).

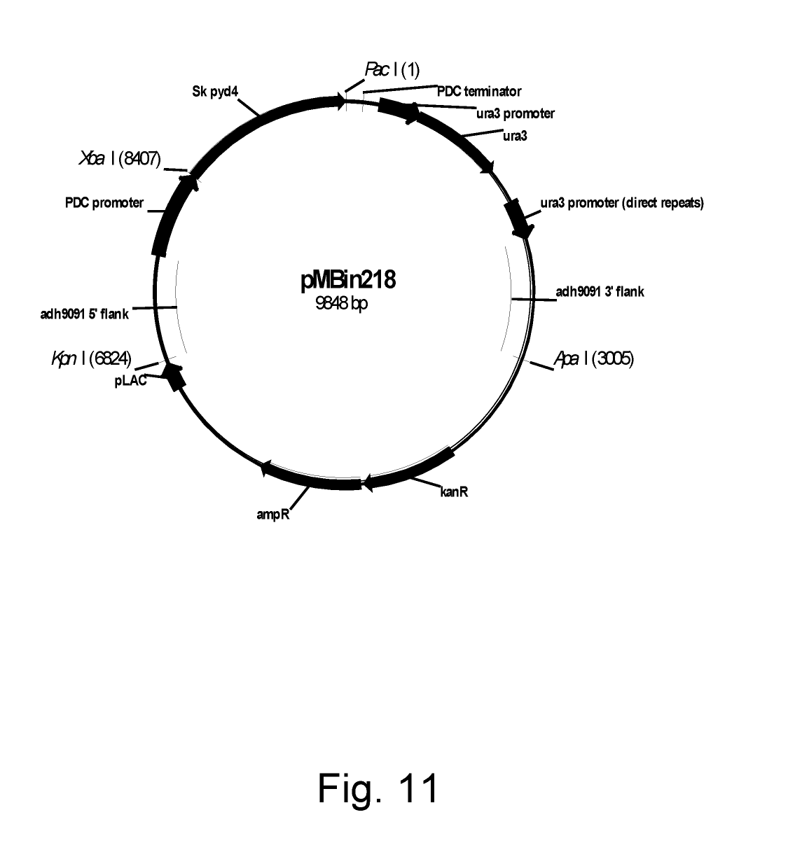

Cloning of the DNA fragment containing the coding sequence of the Saccharomyces kluyveri aminotransferase (supra) into the 8.4 kbp pMBin204 linearized vector (supra) was performed using T4 DNA ligase (New England Biolabs). The reaction mixture contained 1× T4 DNA ligase buffer, 1 μl T4 DNA ligase, 1 μl of the pMBin204 XbaI/PacI digested DNA fragment above, and 7 μl of the 1045171 XbaI/PacI digested product above in total volume of 10 μl. The reaction mixture was incubated at room temperature for at least 1 hour and subsequently used to transform One Shot TOP10 cells (Invitrogen) according to manufacturer's instructions. After a recovery period, two 100 μl aliquots from the transformation mixture were plated onto 150 mm 2×YT plates supplemented with 50 μg of kanamycin per ml and incubated overnight at 37° C. Recombinant colonies of the transformations were each inoculated into 3 ml of LB medium supplemented with kanamycin (50 μg/ml). Plasmid DNA was prepared from these cultures using a BIOROBOT® Universal System (QIAGEN Inc.) and subjected to restriction digest checks. The plasmid DNA from one clone having the correct restriction digest pattern for Saccharomyces kluyveri aminotransferase coding sequence to the I. orientalis adh9091 locus was further subjected to sequence analysis and designated pMBin218 (FIG. 11).

Plasmid pMBin218 contains a Saccharomyces kluyveri aminotransferase coding sequence (SEQ ID NO: 254, which encodes for SEQ ID NO: 21) under control of the PDC promoter and terminator, with the URA3 selectable marker flanked by regions of homology to the adh9091 locus. The aminotransferase of SEQ ID NO: 21 is identified (based on the analysis supra) as a β-alanine aminotransferase (BAAT) and comprises the motif (A,C)-Xn-(GSLS)-Xn-SKA(L/l)(L/l), specifically, (i) AC at a positions corresponding to positions 103-104 of SEQ ID NO: 20; (ii) GSLS at positions corresponding to positions 194-197 of SEQ ID NO: 20; and (iii) SKALI at positions corresponding to positions 354-358 of SEQ ID NO: 20 (See, Table 2 and FIG. 2).

### Example 10: Construction of a Recombinant Strain Expressing a Heterologous Candida arabinofermentans Aminotransferase at the Adh9091 Locus

Strains McTs707 and McTs708 express the heterologous Candida arabinofermentans aminotransferase of SEQ ID NO: 207 at the adh9091 locus, and were constructed as described below.

Approximately 20 μg of pMcTs343 (Example 3) was digested with ApaI, SacI and EheI and separated on a 0.7% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear construct was transformed into yeast strain McTs245 (WO2012074818) as described above.

Several single isolates from each transformation were screened for the site of integration as well as confirming that the other loci were still modified. The integration at adh9091 was confirmed by PCR using LongAmp Taq (New England Biolabs) according to the manufacturer's instructions using primers 614627+612909 and 612908+614626. The PCR product from 612908+614626 yielded an approximately 2.0 kbp band and the product from 614627+612909 yielded an approximately 3.9 kbp band. The adh1202 locus and pyd4 locus were also analyzed to confirm they were still modified using primer sets 611245+612794 (to yield an approximately 3.0 kb band) and 611815+612795 (to yield an approximately 3.6 kb band) for adh1202 locus, and 613242+613243 (to yield an approximately 2.1 kb band) if pyd4 locus deleted. For the integration cassette, two transformants with the correct size bands for the PCRs were designed McTs707 and McTs708.

### Example 11: Construction of a Recombinant Strain Expressing a Heterologous Brettanomyces bruxellensis Aminotransferase or a Heterologous Pichia membranifaciens Aminotransferase at the Adh9091 Locus

Strains McTs761 and McTs762 express the heterologous Brettanomyces bruxellensis aminotransferase of SEQ ID NO: 208 at the adh9091 locus. Strains McTs764 and McTs765 express the heterologous Pichia membranifaciens aminotransferase of SEQ ID NO: 206 at the adh9091 locus. These strains were constructed as described below.

Approximately 40 μg each of pMcTs351 and pMcTs352 (Examples 4 and 5, respectively) was digested with ApaI, SacI and EheI and separated on a 0.7% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear constructs were transformed into yeast strain McTs245 (WO2012074818) as described above.

Several single isolates from each transformation were screened for the site of integration as well as confirming that the other loci were still modified. The integration at adh9091 was confirmed by PCR using LongAmp Taq (New England Biolabs) according to the manufacturer's instructions using primers 614627+612909 and 612908+614626. The PCR product from 612908+614626 yielded an approximately 2.0 kbp band and the product from 614627+612909 yielded an approximately 3.9 kbp band. The adh1202 locus and pyd4 locus were also analyzed to confirm they were still modified using primer sets 611245+612794 (to yield an approximately 3.0 kb band) and 611815+612795 (to yield an approximately 3.6 kb band) for adh1202 locus, and 613242+613243 (to yield an approximately 2.1 kb band) if pyd4 locus deleted. Additionally a primer set specific to either the B. bruxellensis or P. membranifaciens gene were used to confirm the correct integrants. Primer set 614627+1213862 (to yield an approximately 2.0 kb band) confirmed the B. bruxellensis gene at adh9091 and primer set 614627+1213764 (to yield an approximately 2.0 kb band) confirmed the P. membranifaciens gene at adh9091. For each integration cassette, two transformants were selected with the correct size bands by PCR and were designated McTs761 and McTs762 (B. bruxellensis) and McTs764 and McTs765 (P. membranifaciens).

### Example 12: Construction of a Recombinant Strain Expressing a Heterologous Candida guilliermondii Aminotransferase at the Adh9091 Locus

Strains McTs801 and McTs802 express the heterologous Candida guilliermondii aminotransferase of SEQ ID NO: 209 at the adh9091 locus, and were constructed as described below.

Approximately 40 μg of pMcTs356 (Example 6) was digested with ApaI, SacI and EheI and separated on a 0.7% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear construct was transformed into yeast strain McTs245 (WO2012074818) as described above.

Several single isolates from each transformation were screened for the site of integration as well as confirming that the other loci were still modified. The integration at adh9091 was confirmed by PCR using LongAmp Taq (New England Biolabs) according to the manufacturer's instructions using primers 614627+1212900 and 1213770+614626. The PCR product from 614627+1212900 was 1.9 kb and 1213770+614626 was 3.5 kb for correct integrants. The adh1202 locus and pyd4 locus were also analyzed to confirm they were still modified using primer sets 611245+612794 (to yield an approximately 3 kb band) and 611815+612795 (to yield an approximately 3.6 kb band) for adh1202 locus and 613242+613243 (to yield an approximately 2.1 kb band) if pyd4 locus deleted. For the integration cassette, two transformants with the correct size bands for the PCRs was designed McTs801 and McTs802.

### Example 13: Construction of a Recombinant Strain Expressing a Heterologous Candida guilliermondii Aminotransferase at the Adh9091 Locus

Strains McTs697 and McTs698 express the heterologous Candida guilliermondii aminotransferase of SEQ ID NO: 210 at the adh9091 locus, and were constructed as described below.

Approximately 20 μg of pMcTs342 (Example 7) was digested with ApaI, SacI and EheI and separated on a 0.7% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear construct was transformed into yeast strain McTs245 (WO2012074818) as described above.

Several single isolates from each transformation were screened for the site of integration as well as confirming that the other loci were still modified. The integration at adh9091 was confirmed by LongAmp Taq (New England Biolabs) according to the manufacturer's instructions with primers 614627+612909 and 612908+614626. The PCR product from 612908+614626 was 2.0 kb and 614627+612909 was 3.9 kb for correct integrants. The adh1202 locus and pyd4 locus were also analyzed to confirm they were still modified using primer sets 611245+612794 (to yield an approximately 3 kb band) and 611815+612795 (to yield an approximately 3.6 kb band) for adh1202 locus and 613242+613243 (to yield an approximately 2.1 kb band) if pyd4 locus deleted. Additionally PCR primer set 1212904+614626 (to yield an approximately 4.5 kb band) confirmed the C. guilliermondii aminotransferase coding sequence at adh9091. For the integration cassette, two transformants with the correct size bands for the PCRs was designed McTs697 and McTs698.

### Example 14: Construction of a Recombinant Strain Expressing a Heterologous i. Orientalis Aminotransferase at the Adh9091 Locus

Strain MBin533 expresses the heterologous I. orientalis aminotransferase of SEQ ID NO: 20 at the adh9091 locus, and was constructed as described below. The aminotransferase of SEQ ID NO: 20 is identified (based on the analysis supra) as a β-alanine aminotransferase (BAAT) and comprises the motif (A,C)-Xn-(GSLS)-Xn-SKA(L/l)(L/l), specifically, (i) AC at a positions corresponding to positions 103-104 of SEQ ID NO: 20; (ii) GSLS at positions corresponding to positions 194-197 of SEQ ID NO: 20; and (iii) SKALI at positions corresponding to positions 354-358 of SEQ ID NO: 20 (See, Table 2 and FIG. 2).

Approximately 7 μg of pMBin204 (described in WO2012074818) was digested with ApaI, SacI and EheI and separated on a 1.0% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear construct was transformed into yeast strain McTs245 (WO2012074818) as described above.

Several single isolates from each transformation were screened for the site of integration as well as confirming that the other loci were still modified. The integration at adh9091 was confirmed by Phire Plant PCR kit (Thermo) according to the manufacturer's instructions with primers 614180+611678 and 614179+611317. The PCR product from 614180+611678 was 1.1 kb and 614179+611317 was 2.3 kb for correct integrants. The adh1202 locus and pyd4 locus were also analyzed to confirm they were still modified using primer sets 611245+612794 (to yield an approximately 3 kb band) and 611815+612795 (to yield an approximately 3.6 kb band) for adh1202 locus and 612910+612911 (to yield an approximately 1.1 kb band) if pyd4 locus deleted. For the integration cassette, one transformant with the correct size bands for the PCRs was designed MBin533.

### Example 15: Construction of a Recombinant Strain Expressing a Heterologous i. Orientalis Aminotransferase at the Adh9091 Locus

Strain MBin539 expresses the heterologous I. orientalis aminotransferase of SEQ ID NO: 211 at the adh9091 locus, and were constructed as described below.

Approximately 7 μg of pMBin211 (Example 8) was digested with ApaI, SacI and EheI and separated on a 0.7% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear construct was transformed into yeast strain McTs245 (WO2012074818) as described above.

Several single isolates from each transformation were screened for the site of integration as well as confirming that the other loci were still modified. The integration at adh9091 was confirmed by Phire Plant PCR kit (Thermo) according to the manufacturer's instructions with primers 614180+614945 and 614179+611317. The PCR product from 614180+611678 was 1.9 kb and 614179+611317 was 2.5 kb for correct integrants. The adh1202 locus and pyd4 locus were also analyzed to confirm they were still modified using primer sets 611245+612794 (to yield an approximately 3 kb band) and 611815+612795 (to yield an approximately 3.6 kb band) for adh1202 locus. For the integration cassette, one transformant with the correct size bands for the PCRs was designed MBin539.

### Example 16: Construction of a Recombinant Strain Expressing a Heterologous Saccharomyces kluyveri Aminotransferase at the Adh9091 Locus

Strain MBin548 expresses the heterologous Saccharomyces kluyveri aminotransferase of SEQ ID NO: 21 at the adh9091 locus, and were constructed as described below.

Approximately 7 μg of pMBin218 (Example 9) was digested with ApaI, SacI and EheI and separated on a 0.7% agarose gel using 89 mM Tris base-89 mM Boric Acid-2 mM disodium EDTA (TBE) buffer. The desired approximately 6.0 kbp fragment was excised from the gel and purified using NucleoSpin Gel and PCR clean up kit (Macherey-Nagel) according to the manufacturer's instructions. The linear construct was transformed into yeast strain McTs245 (WO2012074818) as described above.

Several single isolates from each transformation were screened for the site of integration as well as confirming that the other loci were still modified. The integration at adh9091 was confirmed by Phire Plant PCR kit (Thermo) according to the manufacturer's instructions with primers 614180+611717 and 614179+611317. The PCR product from 614180+611717 was 1.1 kb and 614179+611317 was 2.5 kb for correct integrants. The adh1202 locus and pyd4 locus were also analyzed to confirm they were still modified using primer sets 611245+612794 (to yield an approximately 3 kb band) and 611815+612795 (to yield an approximately 3.6 kb band) for adh1202 locus and 613243+612911 (to yield an approximately 1.6 kb band) if pyd4 locus deleted. For the integration cassette, one transformant with the correct size bands for the PCRs was designed MBin548.

TABLE 3

| | | | (A,C)-Xn-(GSLS)-Xn- |
|---|---|---|---|
| | | Encoded | SKA(L/I)(L/I) |
| Plasmid | Strain | aminotransferase | Motif |
| pMBin204 | MBin533 | I. orientalis | Yes |
| | | (SEQ ID NO: 20) | |
| pMBin218 | MBin548 | S. kluyveri | Yes |
| | | (SEQ ID NO: 21) | |
| pMBin211 | MBin539 | I. orientalis | No |
| | | (SEQ ID NO: 211) | |
| pMcTs343 | McTs707 | C. arabinofermentans | Yes |
| | McTs708 | (SEQ ID NO: 207) | |
| pMcTs351 | McTs761M | B. bruxellensis | Yes |
| | cTs762 | (SEQ ID NO: 208) | |
| pMcTs352 | McTs764 | P. membranifaciens | Yes |
| | McTs765 | (SEQ ID NO: 206) | |
| pMcTs342 | McTs697M | C. guilliermondii | No |
| | cTs698 | (SEQ ID NO: 210) | |
| pMcTs356 | McTs801 | C. guilliermondii | Yes |
| | McTs802 | (SEQ ID NO: 209) | |

### Example 17: Enzymatic Activity of Recombinant Strains Expressing a Heterologous Aminotransferase

Strains MeJi412 and McTs236 (See, WO2012074818), MBin533, MBin539, McTs708, McTs764, McTs697, and McTs801 were grown in shake flasks according to the following procedure. The strains were streaked out for single colonies on yeast SD-ura is selection plates and incubated at 30° C. for 1-2 days. Seed cultures were prepared in 250 ml baffled flasks containing 50 mL CNB1 shake flask media inoculated with 1-2 colonies from the yeast SD-ura selection plate. The seed cultures were grown for approximately 18 hours at 30° C. with shaking at 200 rpm. Small aliquots of the culture were then withdrawn to measure the OD600 until reaching an OD600 of 4-6. The seed flask cultivation was used to inoculate 125 ml baffled flasks containing 50 mL CNB1 shake flask media to an OD600=0.2. Cultures were incubated at 30° C. with shaking at 140 rpm for 20 hr. An aliquot of the sample was used to measure the optical density (OD) of the culture.

The cells were collected by centrifugation and the supernatant was discarded. For preparation of CFE, the cell pellets were washed with phosphate-buffered saline (PBS) and again collected by centrifugation. The supernatant was discarded and the cell pellet was resuspended to an OD600 of 25 based on the culture OD600 at the time of harvest with a lysis buffer consisting of 1×PBS with an additional 10 mM of pH 7.0 potassium phosphate buffer and Protease Inhibitor Cocktail (Roche 04693159001). 1.8 mL of cell suspension was added to 2.0 mL microcentrifuge tubes pre-filled with approximately 300 μL of 0.5 mm zirconia beads (MP Biomedicals). The cell lysis was performed on a FastPrep®-24 disruptor (MP Biomedicals) for 3 rounds at 6.5 m/s for 50 seconds. Sample tubes were cooled on ice for 3 minutes between each round. After lysis, the samples were centrifuged at 15k RPM in a microcentrifuge for 10 minutes at 4° C. The supernatants were transferred to fresh 1.5 mL tubes in 250 μL aliquots and kept on ice prior to assays or frozen at −20° C. for storage. Total protein concentrations of the lysates were determined via an in-house robotics based BCA assay.

β-Alanine (BAAT) activity and 4-aminobutyrate aminotransferase (gabT) activity in CFE was determined as follows. 190 μL of a reaction mixture containing 100 mM ammonium acetate buffer (pH 6.0 or 7.6), 8 mM α-ketoglutarate, 0.15 mM pyridoxal-5′-phosphate, and 200 mM β-alanine or GABA was added to a 96 well microtiter plate at 25° C. The reaction was initiated by adding 10 μL of CFE. Samples of 20 μL each were taken at 2, 4, 6, 8, 10, and 15 minutes and added to 180 μL of quenching solution (3.0% formic acid). Standards of 0, 0.5, 1.25, 2.5, 5, 10, 20, 40, 80 and 160 g/L glutamate were prepared in 100 mM ammonium acetate (pH 6.0 or 7.6) and 20 μL of these were added to additional wells containing 180 μL of the same quenching solution. Samples were filtered and glutamate quantified via the following LC-MS protocol.

2 μL of sample was injected into an Agilent 1200 series HPLC connected to an Agilent 6410 series Triple Quad LC/MS as detector using an Atlantis columns HILIC Silica 3 μm 2.1×150 mm column (Part no: 186002015). A flow rate of 0.3 ml/min was used with an isocratic gradient of 20% solvent A (95% acetonitrile) and 80% solvent B (0.2% formic acid) with an oven temperature of 50° C. The sample buffer was 20% MeOH+0.1% formic acid+0.2 mg/L C13 labeled glutamic acid (DL-Glutamic acid:H2O (2, 4, 4-d3, 97-98%) Cambridge Isotope, DLM-335-1) Samples were analyzed by Agilent Masshunter Quantitative Analysis software using external standards fit to a linear regression. Deuterium labeled glutamic acid was used as an internal standard for glutamic acid. Results were manually inspected and standards were run in the beginning and the end of the batch with the requirement to be within 5% RSD. Additional mass spectra information and parameters are below:

TABLE 4
Mass spectra acquisition

| | Pre- | Pro- | | | Col- | |
|---|---|---|---|---|---|---|
| | cursor | duct | | | lision | |
| Compound | ion | ion | Dwell | Fragmentor | energy | Polarity |
| Glutamic | 148 | 130 | 50 | 75 | 4 | Positive |
| acid | | | | | | |
| Glutamic | 148 | 102 | 50 | 75 | 7 | Positive |
| acid | | | | | | |
| D₃ Glutamic | 151 | 133 | 50 | 75 | 4 | Positive |
| acid | | | | | | |
| D₃ Glutamic | 151 | 105 | 50 | 75 | 7 | Positive |
| acid | | | | | | |

### QQQ Mass Spectrometer Parameters

Ion source: ESI+(positive ion mode); Stop Mode: no limit/as pump; Stop time: 3.0; Time filter: on; Time filter peak width: 0.1; Delta EMV: 450; Gases: N2; m/z Scan Ranges: 15-1650; Scan mode: Multiple Reaction Monitoring (MRM)

### Ion Source Parameters

Gas temp (° C.): 300; Gas flow (I/min): 10; Nebulizer (psi): 32; Capillary (V): 4000

As shown in Table 5, deletion of the native pyd4 gene results elimination of detectable beta-alanine aminotransferase activity (McTs236 vs. MeJi412). Beta-alanine aminotransferase enzyme activity was restored for strains expressing the heterologous aminotransferases from I. orientalis, C. arabinofermentans, or P. membranifaciens which contain the (A,C)-Xn-(GSLS)-Xn-SKA(L/l)(L/l) motif but not for strains expressing the heterologous aminotransferases I. orientalis and C. guilliermondii aminotransferases which lack the (A,C)-Xn-(GSLS)-Xn-SKA(L/l)(L/l) motif. 4-aminobutyrate aminotransferase enzyme activity was detected for strains expressing the heterologous aminotransferases from I. orientalis or C. guilliermondii which lacks the (A,C)-Xn-(GSLS)-Xn-SKA(L/l)(L/l) motif but not for strains expressing the heterologous aminotransferases from I. orientalis, C. arabinofermentans, or P. membranifaciens which contain the (A,C)-Xn-(GSLS)-Xn-SKA(L/l)(L/l) motif.

TABLE 5

| | | (A,C)-Xn-(GSLS)-Xn- | BAAT Acivity | gabT activity |
|---|---|---|---|---|
| | Encoded | SKA(L/I)(L/I) | (μmol/min/g | (μmol/min/g |
| Strain | aminotransferase | Motif | protein) | protein) |
| MeJi412 | Native pyd4 | Yes | 25.01 | 0.41 |
| McTs236 | Pyd4 deletion | N/A | −1.37 | −2.07 |
| MBin533 | I. orientalis | Yes | 24.86 | 0.19 |
| | (SEQ ID NO: 20) | | | |
| MBin539 | I. orientalis | No | −3.93 | 13.38 |
| | (SEQ ID NO: 211) | | | |
| McTs708 | C. arabinofermentans | Yes | 31.28 | 2.38 |
| | (SEQ ID NO: 207) | | | |
| McTs764 | P. membranifaciens | Yes | 41.95 | 6.06 |
| | (SEQ ID NO: 206) | | | |
| McTs697 | C. guilliermondii | No | −4.62 | 49.00 |
| | (SEQ ID NO: 210) | | | |
| McTs801 | C. guilliermondii | Yes | 13.38 | −3.70 |
| | (SEQ ID NO: 209) | | | |

### Example 18: 3-HP Production of Recombinant Strains Expressing a Heterologous Aminotransferase Comprising the (A,C)-Xn-(GSLS)-Xn-SKA(L/l)(L/l) Motif

Strains MeJi412 and McTs236 (See, WO2012074818), McTs326 (described in WO2012074818), MBin533, MBin548, McTs708, McTs761, McTs764 were grown in shake flasks according the procedure described in Example 18, where the supernatant following centrifugation were the used for 3-HP analysis as described in WO2012074818.

As shown in Table 6, deletion of the native pyd4 gene results in lowered detectable BAAT Activity (McTs236 vs. MeJi412) which was restored (and enhanced) by heterologous expression of an aminotransferase from S. kluyveri, C. arabinofermentans, I. orientalis, B. bruxellensis, or P. membranifaciens containing the (A,C)-Xn-(GSLS)-Xn-SKA(L/l)(L/l) motif. Likewise, deletion of the native pyd4 gene in the control strain MeJi412 (containing active 3-HP pathway) results in lowered detectable 3-HP (McTs236 vs. MeJi412) and 3-HP production was restored (and enhanced) by expressing an aminotransferase from S. kluyveri, C. arabinofermentans, I. orientalis, B. bruxellensis, or P. membranifaciens containing the (A,C)-Xn-(GSLS)-Xn-SKA(L/l)(L/l) motif.

TABLE 6

| | | (A,C)-Xn-(GSLS)- | BAAT Acivity | 3HP production |
|---|---|---|---|---|
| | Encoded | Xn-SKA(L/I)(L/I) | (μmol/min/g | (g/L)/OD600 +/− |
| Strain | aminotransferase | Motif | protein) | Standard deviation |
| MeJi412 | native | Yes | 87.73 | 0.136 +/− 0.0016 |
| McTs236 | PYD4 deletion | N/A | 17.14 | 0.021 +/− 0.0017 |
| MBin533 | I. orientalis | Yes | 119.60 | 0.219 +/− 0.0114 |
| | (SEQ ID NO: 20) | | | |
| MBin548 | S. kluyveri | Yes | 223.61 | 0.211 +/− 0.0153 |
| | (SEQ ID NO: 21) | | | |
| McTs708 | C. arabinofermentans | Yes | 188.07 | 0.178 +/− 0.0153 |
| | (SEQ ID NO: 207) | | | |
| McTs761 | B. bruxellensis | Yes | 122.39 | 0.176 +/− 0.0129 |
| | (SEQ ID NO: 208) | | | |
| McTs764 | P. membranifaciens | Yes | 183.19 | 0.230 +/− 0.0132 |
| | (SEQ ID NO: 206) | | | |

1. A recombinant yeast cell comprising an active 3-HP pathway capable of producing 3-HP and a heterologous polynucleotide encoding an aminotransferase of E.C. 2.6.1.19, wherein:
(a) the aminotransferase comprises the motif (A,C)-Xn-(GSLS)-Xn-SKA(L/l)(L/l);
(b) the aminotransferase has β-alanine aminotransferase (BAAT) activity; and
(c) the aminotransferase is other than a S. kluyveri, I. orientalis, or S. avermitilis aminotransferase.

2. The recombinant cell of claim 1, wherein the aminotransferase comprises:
(i) AC at a positions corresponding to positions 103-104 of SEQ ID NO: 20;
(ii) GSLS at positions corresponding to positions 194-197 of SEQ ID NO: 20; and
(iii) SKA(L/l)(L/l) at positions corresponding to positions 354-358 of SEQ ID NO: 20.

3. A recombinant yeast cell comprising an active 3-HP pathway and a heterologous polynucleotide encoding a β-alanine aminotransferase (BAAT), wherein the BAAT has at least 80% sequence identity to SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234; and wherein the cell capable of producing 3-HP.

4. The recombinant cell of claim 3, wherein the β-alanine aminotransferase (BAAT) comprises the motif (A,C)-Xn-(GSLS)-Xn-SKA(L/l)(L/l).

5. The recombinant cell of claim 4, wherein the β-alanine aminotransferase (BAAT) comprises:
(i) AC at a positions corresponding to positions 103-104 of SEQ ID NO: 20;
(ii) GSLS at positions corresponding to positions 194-197 of SEQ ID NO: 20; and
(iii) SKA(L/l)(L/l) at positions corresponding to positions 354-358 of SEQ ID NO: 20.

6. The recombinant cell of claim 3, wherein the β-alanine aminotransferase (BAAT) has an amino acid sequence comprising or consisting of SEQ ID NO: 206, SEQ ID NO: 207, SEQ ID NO: 208, SEQ ID NO: 209, SEQ ID NO: 218, SEQ ID NO: 220, SEQ ID NO: 222, SEQ ID NO: 224, SEQ ID NO: 226, SEQ ID NO: 228, SEQ ID NO: 230, SEQ ID NO: 232, or SEQ ID NO: 234.

7. The recombinant cell of claim 3, wherein the heterologous polynucleotide comprises a coding sequence having at least 80% sequence identity to SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251.

8. The recombinant cell of claim 3, wherein the heterologous polynucleotide has a coding sequence that comprises or consists of SEQ ID NO: 239, SEQ ID NO: 240, SEQ ID NO: 241, SEQ ID NO: 242, SEQ ID NO: 243, SEQ ID NO: 244, SEQ ID NO: 245, SEQ ID NO: 246, SEQ ID NO: 247, SEQ ID NO: 248, SEQ ID NO: 249, SEQ ID NO: 250, or SEQ ID NO: 251.

9. The recombinant cell of claim 1, wherein the aminotransferase lacks an N-terminal mitochondrial target sequence (MTS).

10. The recombinant cell of claim 1, wherein the active 3-HP pathway proceeds through a β-alanine and/or malonate semialdehyde intermediate.

11. The recombinant yeast cell of claim 1, wherein the cell produces a greater amount of 3-HP compared to the cell without the heterologous polynucleotide encoding the β-alanine aminotransferase (BAAT), when cultivated under identical conditions (e.g., at least 10%, at least 15%, at least 20%, at least 25%, at least 30%, at least 35%, at least 40%, at least 45%, at least 50%, at least 75%, at least 100%, at least 150%, at least 200%, at least 250%, at least 300%, or at least 500% more).

12. The recombinant cell of claim 1, wherein the cell belongs to a genus selected from Candida, Kluyveromyces, Pichia, Issatchenkia, Schizosaccharomyces, Torulaspora, Zygosaccharomyces, and Saccharomyces.

13. The recombinant cell of claim 12, wherein the cell is selected from I. orientalis, C. lambica, and S. bulderi.

14. The recombinant cell of claim 1, wherein the cell is a CNB1 yeast cell.

15. A composition comprising the recombinant cell of claim 1, and a fermentable medium.

16. A method of producing 3-HP, comprising:
(a) cultivating the recombinant yeast cell of claim 1 in a fermentable medium under suitable conditions to produce 3-HP; and
(b) recovering the 3-HP.

17. A method of producing acrylic acid or a salt thereof, comprising:
(a) cultivating the recombinant cell of claim 1 in a fermentable medium under suitable conditions to produce 3-HP;
(b) recovering the 3-HP;
(c) dehydrating the 3-HP under suitable conditions to produce acrylic acid or a salt thereof; and
(d) recovering the acrylic acid or salt thereof.

18. A method of producing a 3-HP ester, comprising:
(a) cultivating the recombinant cell of claim 1 in a fermentable medium under suitable conditions to produce 3-HP;
(b) recovering the 3-HP;
(c) esterifying the 3-HP under suitable conditions to produce a 3-HP ester; and
(d) recovering the 3-HP ester

19. A method of producing 1,3-propanediol, comprising:
(a) cultivating the recombinant cell of claim 1 in a fermentable medium under suitable conditions to produce 3-HP;
(b) recovering the 3-HP;
(c) hydrogenating the 3-HP under suitable conditions to produce 1,3-propanediol; and
(d) recovering the 1,3-propanediol.

20. The method of claim 16 wherein the recombinant cell is a CNB1 cell cultivated in a fermentable medium comprising less than 1% pentose sugars.
